(19) United States
(12) Patent Application Publication
(10) Pub. No.: US 2024/0398997 A1
(43) Pub. Date: Dec. 5, 2024

(54) BIOLUMINESCENT PROBES FOR VISUALIZING RNA DYNAMICS

(71) Applicant: THE REGENTS OF THE UNIVERSITY OF CALIFORNIA, Oakland, CA (US)

(72) Inventors: Jennifer A. Prescher, Irvine, CA (US); Andrej Luptak, Irvine, CA (US); Oswald Steward, Irvine, CA (US); Kevin K. Ng, Irvine, CA (US); Lila P. Halbers, Irvine, CA (US); Kyle Cole, Irvine, CA (US); Claire C. Chen, Irvine, CA (US); Erin B. Fuller, Irvine, CA (US); Christelle E. T. Chan, Irvine, CA (US)

(21) Appl. No.: 18/680,929

(22) Filed: May 31, 2024

Related U.S. Application Data
(60) Provisional application No. 63/505,116, filed on May 31, 2023.

(51) Int. Cl. A61K 49/00

(52) U.S. Cl. CPC A61K 49/0054; A61K 49/0045

(57) ABSTRACT

A complete understanding of RNA biology requires methods for tracking transcripts in vivo. Common strategies rely on fluorogenic probes that are limited in sensitivity, dynamic range, and depth of interrogation, owing to their need for excitation light and tissue autofluorescence. To overcome these challenges, a bioluminescent platform for serial imaging of RNAs is described herein. Small RNA tags were engineered to recruit light-emitting luciferase fragments (termed RNA lanterns) upon transcription. Robust photon production was observed for RNA targets both in cells and in live animals. Importantly, only a single copy of the tag was necessary for sensitive detection, in sharp contrast to fluorescent platforms requiring multiple repeats. Overall, this work provides a foundational platform for visualizing RNA dynamics from the micro to the macro scale.

### CROSS-REFERENCE TO RELATED APPLICATIONS

This application is a non-provisional and claims benefit of U.S. Provisional Application No. 63/505,116 filed May 31, 2023, the specification of which is incorporated herein in their entirety by reference.

### STATEMENT REGARDING FEDERALLY SPONSORED RESEARCH OR DEVELOPMENT

This invention was made with government support under Grant No. 1804220 awarded by National Science Foundation, Grant No. R01CA229696 awarded by National Institute of Health, and ICAR 80NSSC21 K0596 award by National Aeronautics and Space Administration. The government has certain rights in the invention.

### REFERENCE TO AN ELECTRONIC SEQUENCE LISTING

The contents of the electronic sequence listing (UCI_22_18_NP.xml; Size: 88,258 bytes; and Date of Creation: May 29, 2024) is herein incorporated by reference in its entirety.

### FIELD OF THE INVENTION

The present invention features methods and compositions for visualizing RNA dynamics.

### BACKGROUND OF THE INVENTION

RNA dynamics play pivotal roles in a multitude of cellular processes. While there is a deep, molecular-level understanding of many facets of RNA biology in vitro, the picture in physiologically authentic environments—live animals—remains incomplete. This is due, in part, to a lack of methods for noninvasive tracking of RNAs in vivo. Conventional approaches rely on RNA tags coupled with fluorescent probes. Such platforms require excitation light, which can be challenging to deliver in whole organisms without invasive procedures, excision of tissues, or delivery of fluorogenic dyes. Furthermore, external light can induce autofluorescence, precluding sensitive detection of low abundance targets. Short imaging times are also necessary to avoid light-induced damage. Consequently, in live mammals, tracing the lifecycle of critical RNAs in real time has been elusive.

Thus, the present invention features a more suitable platform for RNA imaging in live animals with bioluminescence. This modality relies on photon production from luciferase enzymes and luciferin small molecules. Since no excitation light is required, bioluminescence can provide superior signal-to-noise ratios in vivo for visualization of low-copy transcripts. Additionally, serial imaging is possible without concern for phototoxicity or tissue damage.

### BRIEF SUMMARY OF THE INVENTION

It is an objective of the present invention to provide compositions and methods that allow for visualizing RNA dynamics in vivo, as specified in the independent claims. Embodiments of the invention are given in the dependent claims. Embodiments of the present invention can be freely combined with each other if they are not mutually exclusive.

The present invention features a general method that leverages advances in bioluminescence technology for multi-scale RNA detection. The approach features split fragments of NanoLuc (NanoBiT) fused to MS2 and PP7 bacteriophage coat proteins (MCP and PCP), fusions that the Inventors have termed RNA lanterns. MCP and PCP bind distinct RNA aptamers (MS2 and PP7, respectively) that can be appended to transcripts of interest. Upon transcription, MCP and PCP bind the MS2-PP7 containing RNA bait, bringing the luciferase fragments into proximity to assemble a functional, light-emitting enzyme. The lanterns and RNA bait were extensively optimized to maximize signal turn-on and minimize the size of the protein-RNA complex. Notably, a single rigidified RNA was sufficient for sensitive imaging both in cells and in vivo, rendering much larger RNA tags unnecessary. Additionally, the RNA bait is modular and can be used in conjunction with other split luciferases and for multi-scale imaging. The tools reported here are thus immediately valuable for studies of RNA dynamics.

In some embodiments, the present invention features a bioluminescent composition (i.e., RNA lantern) for visualizing RNA dynamics. In some embodiments, the composition comprises an RNA (e.g., a structured RNA) comprising at least a first stem-loop, wherein the first stem-loop may be bound by a first RNA-interacting protein linked to a first portion of a detectable reporter. In other embodiments, the composition comprises an RNA (e.g., a structured RNA) comprising a first stem-loop that may be bound by a first RNA-interacting protein linked to a first portion of a detectable reporter and a second stem-loop that may be bound by a second RNA-interacting protein linked to a second portion of the detectable reporter. In further embodiments, the composition comprises an RNA (e.g., a structured RNA) comprising a first stem-loop that may bound by a first RNA-interacting protein linked to a first portion of a detectable reporter, a second stem-loop that may be bound by a second RNA-interacting protein linked to a second portion of the detectable reporter, and a third stem-loop that may be bound to a second detectable reporter. In some embodiments, the third stem-loop is downstream of the first stem-loop and/or the second stem-loop.

In some embodiments, the detectable reporter (e.g., the first detectable reporter and/or the second detectable reporter) comprises an optical reporter. In some embodiments, the detectable reporter (e.g., the first detectable reporter and/or the second detectable reporter) comprises a fluorescent reporter. Non-limiting examples of fluorescent reporters include yellow fluorescent proteins (YFP), red fluorescent proteins (RFP), or green fluorescent proteins (GFP). In some embodiments, the detectable reporter (e.g., the first detectable reporter and/or the second detectable reporter) comprises a light-emitting luciferase protein. In some embodiments, the detectable reporter comprises a proximity labeling protein.

In some embodiments, the first portion of the detectable reporter comprises a first portion of a light-emitting luciferase protein (e.g., the N-terminal fragment of the light-emitting luciferase protein). In some embodiments, an MS2 RNA binding protein (e.g., the first RNA-interacting protein) is linked to the N-terminal fragment of the light-emitting luciferase protein. In certain embodiments, the MS2 RNA binding protein is linked to the N-terminal fragment via a linker. In some embodiments, the second portion of the detectable reporter comprises a second portion of the light-emitting luciferase protein (e.g., the C-terminal fragment of the light-emitting luciferase protein). In some embodiments, the PP7 RNA binding protein (e.g., the second RNA-interacting protein) is linked to the C-terminal fragment of the light-emitting luciferase protein. In certain embodiments, the PP7 RNA binding protein is linked to the C-terminal fragment via a linker. In some embodiments, the detectable reporter (e.g., the first detectable reporter) becomes functional when the first portion and the second portion are brought together.

In some embodiments, the 5′ end of the RNA and the 3′ end of the RNA are complementary and form an RNA stem. In such embodiments, the first stem-loop and the second stem-loop may be between the complementary 5′ end and the 3′ end of the RNA. In other embodiments, the 5′ end of the RNA and the 3′ end of the RNA are non-complementary and form single-stranded RNA (ssRNA) arms. In such embodiments, the ssRNA arms may flank the first stem-loop and the second stem-loop. In some embodiments, the bioluminescent composition may be unstructured, for example, the RNA may be unstructured until a target RNA base pairs with the ssRNA arms. For example, when the target RNA binds to the ssRNA arms, a structure RNA is formed. A structure RNA may refer to an RNA comprising extensive base-pairing, tertiary interactions, quaternary interactions, or a combination thereof.

In some embodiments, the target RNA comprises a cellular RNA, e.g., a messenger RNA (mRNA), a splice variant of an mRNA, a microRNA, or a non-coding RNA (ncRNA).

In some embodiments, the bioluminescent composition (RNA lantern) comprises a structured RNA formed by trans-splicing, RNA editing, or DNA editing. In some embodiments, a structure RNA refers to an RNA comprising extensive base pairing, tertiary interactions, or a combination thereof.

One of the unique and inventive technical features of the present invention is the use of a structured RNA. Without wishing to limit the invention to any theory or mechanism, it is believed that the technical feature of the present invention advantageously provides more efficient reporter detection, enabling ultra small tags to be used. None of the presently known prior references or works have the unique inventive technical feature of the present invention.

Moreover, the prior references teach away from the present invention. For example, the rigidified RNA tags described herein provided superior signal-to-noise ratios and enabled the smallest tags for transcript imaging to date. Previous RNA designs incorporated RNA aptamers that bind MS2 and PP7 proteins, but the aptamers were linked via flexible linkers. The present work shows that optimized rigidified RNAs greatly enhance the complexation of the split luciferase. Furthermore, flexible linkers require multiple copies of the aptamers to be used to generate complexes with strong enough signal to distinguish form background fluorescence of cells. However, because both the MS2 and PP7 proteins are derived from phage coats, the proteins assemble into capsids and form very large complexes that may interfere with the function of the targeted RNA.

Furthermore, the inventive technical features of the present invention contributed to a surprising result. For example, the kinetics of RNA production can be directly observed, because the luciferase enzymes become functional immediately upon complex formation. This means that photon production begins as soon as the proteins assemble on the RNA and the first signal is observable within seconds of the RNA synthesis. This observation is in stark contrast with fluorescent protein detection, which often depends on maturation rates of the protein-bound fluorophore and typically takes tens of minutes to hours, precluding the observation of real-time dynamics of RNA synthesis.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIGS. 1A, 1B, 1C, 1D, 1E, and 1F shows an optimized platform for tracking RNA dynamics. FIG. 1A shows the overall strategy to visualize transcripts using RNA lanterns. The lanterns comprise fusions of MS2 coat protein (MCP) and PP7 coat protein (PCP) with NanoBiT fragments (SmBiT and LgBiT, respectively). Transcription of bait RNA (comprising MS2 and PP7 aptamers) drives NanoBiT heterodimerization. In the presence of a luciferin substrate (furimazine, FRZ), light is produced (blue glow). The bicistronic construct encoding the RNA lantern is shown below the scheme. MCP and PCP were fused with HA and FLAG tags, respectively, for expression analyses. FIG. 1B shows a modeling of the RNA lantern complex. Crystal structures of MS2 (purple, 1ZDI), PP7 (orange, 2QUX), and NanoLuc (5IBO), highlighting LgBiT (dark blue) and SmBiT (cyan), were modeled in ChimeraX. The MCP-SmBiT/PCP-LgBiT complex was modeled by aligning the corresponding N- and C-termini of each protein and aligning the 5′ and 3′ ends of the aptamers. FIG. 1C shows a predicted secondary structure of flexible RNA bait, as calculated by RNAfold. FIG. 1D shows the initial tests of RNA lanterns with a flexible RNA bait via an in vitro transcription and translation (IVTT) assay. The total flux observed in the absence or presence of RNA bait is plotted. FIG. 1E shows engineered rigid RNA baits providing robust photon output in IVTT assay. RNA baits containing varying spacers between the MS2 and PP7 aptamers were constructed, with spacer length (X nt, blue, denoted below each bar graph), modulating the distance and helical phase angle between the aptamers. Fold change in signal over RNA lantern alone is plotted. MS2, PP7, and M-3-P^{mut} denote baits comprising isolated aptamers or a mutated PP7 aptamer (FIGS. 4A and 4B), respectively, none of which were expected to result in RNA lantern assembly. Representative luminescence images are shown below the graph. FIG. 1F shows rigid RNA baits comprising various MS2 and PP7 stem lengths (top right, purple, and orange for the MS2 and PP7 aptamers, respectively). Luminescence readouts were acquired following IVTT. Fold change in signal over RNA lantern alone is plotted. Representative luminescence images are shown below the graph. Error bars represent the standard error of the mean (SEM) for n=2 replicates (FIG. 1D) and n=4 replicates (FIGS. 1C, 1E, and 1F).

FIGS. 2A, 2B, and 2C show the optimization of the split NanoLuc RNA lantern. FIG. 2A shows SmBiThigh (SmBiT99) and SmBiTlow (SmBiT114), and their serial trimers were constructed as NLS-MCP fusions and co-expressed with IRES-driven PCP-LgBiT. FIG. 2B shows the lantern constructs were expressed in IVTT (rabbit reticulocyte lysate, RRL) and evaluated for RNA-independent photon production alone or supplemented with synthetic SmBiTultra (SmBiT86, 10 μM) or recombinant LgBiT (10 μM), as noted. The IVTT-expressed lanterns provide background photon measurements, whereas the supplemented experiments (+SmBiTultra or +LgBiT) represent the saturated complexes, reporting the maximum potential photon output from each construct. FIG. 2C shows the fold change in light output from FIG. 2B, calculated for pairs of (saturated/IVTT-expressed) constructs. Error bars represent the standard error of the mean (SEM) for n=3 replicate experiments.

FIGS. 3A and 3B show the design of novel MCP/PCP baits. FIG. 3A shows the RNA baits were designed with varying numbers of nucleotides between the MS2 (purple) and PP7 (orange) aptamers. A Ni-NTA aptamer (green) was also added for purification purposes and improved structural rigidity. FIG. 3B shows luminescence readouts obtained with each RNA bait. Data are plotted as the fold change in signal from each RNA bait over a no-RNA control sample. Decreasing concentrations of RNA bait templates were assessed with 1 ng of the lantern DNA plasmid. The mean fold change for each sample (n=2) is shown.

FIGS. 4A, 4B, and 4C show the lack of luminescence observed with control RNAs. FIG. 4A shows the predicted secondary structures of control RNA baits comprising individual MS2 or PP7 aptamers or the M-3-Pmut construct containing two mutations (deletion of A6 and G10). FIG. 4B shows the location of the deleterious PP7 mutations in the structure of the unmutated aptamer-protein complex (PDB: 2QUX). FIG. 4C shows the fold change in signal observed with each control RNA bait over a no-RNA sample. Decreasing concentrations of RNA bait templates were tested with 1 nM of the lantern DNA plasmid. The mean fold change for each sample (n=2) is shown.

FIGS. 5A and 5B show modulation of aptamer stem lengths. FIG. 5A shows the stem regions of the MS2 (purple) and PP7 (orange) aptamers were varied by changing the number of base pairs. The asterisk indicates RNA bait with unmodified MS2 and PP7 stems (FIG. 1E). FIG. 5B shows the fold change in signal observed with each RNA bait design over a no-RNA control. Decreasing concentrations of each RNA bait template were tested with 1 ng of the lantern DNA plasmid. The mean fold change for each sample (n=2) is shown.

FIGS. 6A and 6B show aptamer order is critical for lantern assembly. FIG. 6A shows the PP7 aptamer (orange) was placed either 5′ (right) or 3′ (left) of the MS2 aptamer (purple) to evaluate the effect of binding site orientation on lantern function. FIG. 6B shows RNA baits were assessed using IVTT and the fold changes in light output over samples without RNA bait are plotted. Error bars represent the standard error of the mean (SEM) for n=4 replicates.

FIGS. 7A, 7B, 7C, 7D, and 7E show lantern linker lengths have a smaller effect on photon output than RNA bait structures. FIG. 7A shows schematic of G4S linker changes in the RNA lantern. FIG. 7B shows protein linkers comprising 1-5 copies of a glycine-serine repeating unit (G4S) were examined in IVTT. Luminescence outputs were recorded in the presence of various RNA baits. Data are plotted as the fold change in signal over no-RNA bait controls. FIG. 7C shows fold change in luminescence observed with RNA lanterns comprising altered linkers and M-3-P versus the original lantern and M-3-P. No substantial increase in signal was observed with probes comprising G4S units. For FIGS. 7A and 7B, error bars represent the standard error of the mean (SEM) for n=4 replicates. FIG. 7D shows a schematic of an mRNA encoding blue fluorescent protein (BFP) and M-3-P in the 3′ UTR. Transcription of BFP-M-3-P mRNA recruits the RNA lantern, resulting in light production. BFP fluorescence enables confirmation of expression. FIG. 7E shows RNA lanterns comprising various linker lengths were examined. Cells stably expressing each lantern were transfected with the BFP-M-3-P construct. Data are plotted as the fold-change in signal over non-transfected cells. Luminescence measurements were normalized to BFP expression (assessed via flow cytometry). Nine replicates are shown, and the error bars represent the standard error of the mean (SEM) for all replicates. Minimal improvement over the initial RNA lantern design was observed with varying glycine-serine linkers. The 5×-5× design (five G4S linkers in both protein fusions) exhibited reduced signal turn-on both in vitro and in cellulo, suggesting disfavored complementation with increasing linker length.

FIGS. 8A, 8B, 8C, 8D, 8E, and 8F shows Biochemical validation of RNA lanterns. FIG. 8A shows the designer RNA bait comprises a 4-way-junction of MS2, PP7, and Ni-NTA aptamers. This unit assembles the RNA lantern components (MCP-SmBiT and PCP-LgBiT, fused to HA and FLAG epitopes, respectively). FIG. 8B shows bioluminescent output from RNA bait (M-3-P) or inactive mutant (M-3-P^{mut}) combined with lysate from cells stably expressing the RNA lantern. FIG. 8C shows fold change in bioluminescent signal over no-RNA controls. Each bar represents the median of n=3 replicates with dots showing data from individual replicates. FIG. 8D shows affinity purification of the RNA lantern complex using the Ni-NTA aptamer. Various concentrations of RNA bait were used, and captured complexes were eluted using a metal chelator (EDTA). FIG. 8E shows the bioluminescent output of lantern complexes captured using anti-HA or anti-FLAG conjugated-agarose beads. FIG. 2F shows fold change in signal from lantern complexes retrieved in the presence of M-3-P bait versus no RNA from (FIG. 8E). Error bars represent the standard error mean (SEM) of n=3 replicates. BL, bioluminescence. a.u., arbitrary units.

FIGS. 9A and 9B shows the kinetics of RNA lantern assembly. FIG. 9A shows bioluminescent output from RNA bait (M-3-P, red), inactive mutant (M-3-P^{mut}, black), or no bait (gold). Samples were mixed with lysate from cells expressing RNA lanterns and analyzed over time. Two replicate experiments were performed for each condition, and all runs are shown. FIG. 9B shows real-time RNA lantern assembly with purified RNA bait, along with RNAs produced via in vitro transcription. Lanterns were expressed by in vitro transcription and translation in reticulocyte lysate prior to the addition of either purified M-3-P RNA bait (1 nM), M-3-P DNA (1 nM), or GFP-M-3-P DNA (1 nM). Luminescence was measured every 30 s, and mean luminescence values (from n=3 replicates) were plotted. Data were normalized to the maxima at 35 min for each 30 s interval. Solid curves are data fit to the mono-exponential function y=1−e^{-k(t-t}^{₀}⁾.

FIGS. 10A, 10B, 10C, 10D, 10E, 10F, 10G, and 10H shows Robustness and modularity of structured RNA baits. FIG. 10A shows a comparison of M-3-P to a 12-copy unstructured RNA bait. Top: Schematic of the structured M-3-P RNA bait (single copy). Bottom: Schematic of a flexible RNA bait comprising 12 copies of the MS2 and PP7 aptamers (MP_{12X}). FIG. 10B shows MP₁₂ₓ was evaluated against M-3-P at equimolar RNA concentrations (1 nM of template DNA) or at 1/12 the concentration (83 pM of template DNA). Fold change over no-RNA bait samples is plotted. FIG. 10C shows RNA bait assembles other lanterns. Schematic of MCP-PCP probes fused to split firefly luciferase (Fluc). MCP is fused to the N-terminal half of Fluc and PCP is fused to the C-terminal half. RNA transcription followed by D-luciferin treatment enables photon production and visualization. FIG. 10D shows the Fluc lantern was assessed with a panel of RNA baits. Total light output for n=6 replicates is shown. Only 2 replicates are shown for the no-RNA bait sample; the others were below the limit of detection represented by the electronic noise of the EMCCD camera (dashed line). FIG. 10E shows a graph of fold change in signal over no-RNA bait samples from (FIG. 10D). FIG. 10F shows a schematic of BRET-based RNA lanterns. FIG. 10G shows luminescence fold change measurements for yellow and red RNA lanterns across a panel of RNA baits. Fold change calculated over no-RNA controls. FIG. 10H shows emission spectra for BRET-based RNA lanterns. Error bars represent the standard error of the mean (SEM) for n=12 replicates (FIG. 9B), n=6 replicates (FIGS. 9D and 9E), n=10 replicates (FIG. 9G), and n=3 replicates (FIG. 9H). a.u., arbitrary units.

FIGS. 11A, 11B, 11C, and 11D show Dynamic imaging in mammalian cells with RNA lanterns. FIG. 11A shows a schematic of model mRNA encoding a fluorescent protein. The sequence was engineered with M-3-P in the 3′ UTR. Transcription of XFP-M-3-PmRNA results in lantern assembly and light emission (blue glow). XFP production can be further analyzed via fluorescence (green glow). FIG. 11B shows HEK293T cells expressing the RNA lantern were transiently transfected with DNA encoding GFP-M-3-P (Pearson's r=0.84) or GFP alone (Pearson's r=0.03). Luminescence was observed exclusively in cells containing mRNAs with the M-3-P bait. Scale bar=20 μm. FIG. 11C shows bulk measurement of photon flux from lantern-expressing cells transfected with DNA encoding GFP or GFP-M-3-P. FIG. 11D shows a schematic for mRNA imaging during cellular stress. FIG. 11E shows imaging mRNA localization. HEK293T cells expressing RNA lanterns were transfected with DNA encoding mCherry-β-actin (with M-3-P located in the 3′ UTR). Cells were treated with sodium arsenite and imaged before and after treatment (0 and 50 min, respectively). Fluorescence readouts (mCherry) confirmed successful reporter transfection and expression. Luminescence images show the appearance of distinct puncta over time. Scale bars=20 μm. FIG. 5F shows mRNA localizes to stress granules. HEK293T cells expressing RNA lanterns were transfected with mCherry-β-actin (with M-3-P located in the 3′ UTR) and GFP-G3BP1. Cells were treated with sodium arsenite and imaged as in (B). GFP-G3BP1 is known to localize to stress granules. Scale bars=20 μm. FIG. 5G shows magnified views of two cell clusters from (C). Scale bars=20 μm.

FIGS. 12A, 12B, 12C, and 12D shows imaging in live mice with RNA lanterns. FIG. 12A shows mice implanted with cells expressing RNA lanterns and GFP alone (left flank) or GFP-M-3-P (right flank) and imaged with luciferin. FIG. 12B shows photon flux from mice with implanted cells expressing GFP(±)M-3-P and RNA lanterns shown in (FIG. 12D). FIG. 12C shows mice implanted with cells expressing RNA lanterns and BFP-M-3-P^{mut} (left flank) or BFP-M-3-P (right flank). FIG. 12D shows photon flux from mice with implanted cells expressing either BFP-M-3-P or BFP-M-3-P^{mut} and RNA lanterns. Error bars represent standard deviation (SD) for n=3 replicates (FIGS. 10C, 10E, and 10G). P-values (95% confidence interval) were determined by unpaired, two-tailed t-test. BL, bioluminescence. a.u., arbitrary units.

FIG. 13 shows RNA imaging in cellulo with bioluminescent lanterns. HEK293T cells expressing RNA lanterns were transfected with DNA encoding GFP-M-3-P (Pearson's r=0.57) or GFP alone (Pearson's r=0.15). The images shown are replicates of the experiment described in FIG. 10B. Scale bar=20 μm.

FIG. 14 shows the imaging of RNA lanterns in mammalian cells. HEK293T cells expressing RNA lanterns were transfected with DNA encoding GFP-M-3-P. Luminescence was observed in cells containing mRNAs with the M-3-P bait using both a 10× and 4× objective. Scale bars=80 μm.

FIGS. 15A. 15B, 15C, 15D, and 15E show the quantification of RNA lantern complexes in mammalian cells. FIG. 15A shows the bulk bioluminescent outputs from HEK293T cells expressing RNA lanterns. Cells were transfected with DNA encoding GFP-M-3-P (or no DNA) and analyzed. Error bars represent the standard deviation (SD) of the mean for n=4 replicates. FIG. 15B shows the bioluminescence of RNA lantern complexes captured from homogenized cells from FIG. 15A using the Ni-NTA aptamer. FIG. 15C shows the fold change in bioluminescent signal from RNA lantern complexes retrieved in the presence of GFP-M-3-P versus no mRNA from FIGS. 15A and 15B). FIG. 15D shows the concentrations of GFP-M-3-P mRNA from cell lysate (open circles) and GFP-M-3-P mRNA (closed squares) captured on Ni-NTA resin from FIG. 15B. Values were determined using real-time quantitative PCR (qPCR). FIG. 15E shows the standard curve from qPCR in FIG. 15D, with ten-fold serial dilutions of GFP-M-3-P cDNA (10-14-10-9 M). Data were used to establish the quantification cycle (Cq) relative to log[RNA]. Duplicate measurements were taken and are both displayed. BL, bioluminescence. a.u., arbitrary units.

FIGS. 16A, 16B, and 16C show live-cell imaging of gene expression. FIG. 16A shows a schematic of doxycycline (dox)-inducible mRNA production. The reporter construct comprised a Tet-on repressor element (TRE2) upstream of a sequence encoding mRFP (XFP) with M-5-P in the 3′ UTR. The addition of dox promotes transcription. FIG. 16B shows bioluminescent output from an inducible model. HEK293T cells were transfected with plasmids encoding RNA lanterns and TRE2-XFP-M-5-P. Bioluminescence was measured for ˜1 h (90 s acquisitions, 71 frames) post-dox addition. Fluorescence images were acquired at 10 min and ˜2-h post-dox addition. Scale bars=20 μm. FIG. 16C shows luminescent outputs from individual cells (#1-5) analyzed over time.

FIGS. 17A and 17B show a schematic for mRNA imaging during cellular stress. FIG. 17A shows imaging mRNA localization. HEK293T cells expressing RNA lanterns were transfected with DNA encoding mCherry-β-actin (with M-3-P located in the 3′ UTR). Cells were treated with sodium aresenite and imaged before and after treatment (0 and 50 min, respectively). Fluorescence readouts (mCherry) confirmed successful reporter transfection and expression. Luminescence images show the appearance of distinct puncta over time. Scale bars=20 μm. FIG. 17B shows mRNA localizes to stress granules. HEK293T cells expressing RNA lanterns were transfected with mCherry-β-actin (with M-3-P located in the 3′ UTR) and GFP-G3BP1. Cells were treated with sodium arsenite and imaged as in (B). GFP-G3BP1 is known to localize to stress granules. Scale bars=20 μm. (G) Magnified views of two cell clusters from (C). Scale bars=20 μm.

FIG. 18 shows G3BP1 expression in stressed cells. HEK293T cells stably expressing RNA lanterns were transfected with DNA encoding CDK6-M-3-P-IRES-GFP. Some cells were treated with arsenite (+) to induce cell stress. Cells were fixed and stained with anti-G3BP1 or no antibody (secondary only), followed by a fluorescent secondary antibody. Fluorescence images are shown. Scale bars=20 μm.

FIGS. 19A and 19B shows live-cell imaging of CDK6 during cellular stress. FIG. 19A shows a schematic of mRNA trafficking to stress granules upon arsenite treatment. FIG. 19B shows dynamic imaging of mRNAs. HEK293T cells expressing RNA lanterns were transfected with DNA encoding CDK6-M-3-P-IRES-GFP. Cells were treated with arsenite as in FIGS. 17A and 17B and imaged over time. Fluorescence readouts confirmed successful reporter transfection and expression. Luminescence images showed increased granule formation over time. Scale bars=20 μm. Stress granule formation was confirmed by staining for G3BP1 expression.

FIGS. 20A, 20B, and 20C show next-generation RNA lanterns and mini-tags for multiplexed transcript visualization in vivo. FIG. 20A shows that BRET-based lanterns provide red-shifted light. FIG. 20B shows emission spectra from NanoLuc (Blue) fused to YFP (Yellow) or mScarlet-I (Red). Red-emitting photons (>600 nm) are desirable for improved tissue penetrance. FIG. 20C shows orthogonal lanterns for multi-transcript imaging require alternate RNA motifs and cognate binding domains (gold).

FIGS. 21A, 21B, and 21C show red-shifted lanterns for improved tissue penetrance. FIG. 21A shows yellow and red-emitting lanterns derived from fluorescent protein fusions to LgBiT. Red-shifted light is produced via BRET. FIG. 21B shows yellow and red lanterns produce more red-shifted light in the presence of the RNA mini-tag. (The residual emission peak at 460 nm is due to incomplete BRET transfer, common to these fusions). FIG. 21C shows red lanterns enable imaging though deeper tissue (mm of turkey bacon shown as model tissue).

FIGS. 22A, 22B, and 22C show orthogonal lantern development. FIG. 22A shows λN and boxB comprise another lantern/mini-tag combination. PDB: 1HJI, PDB: 1RLG. FIG. 22B shows a cartoon representation of the orthogonal probe set. FIG. 22C shows MS2-boxB mini-tag can be readily distinguished from MS2-PP7 in the presence of yellow and blue lanterns, respectively.

FIG. 23 shows a palette of RNA lanterns and complementary mini-tags. RNA tags will be optimized in terms of hairpin spacing and stem lengths.

FIG. 24 shows measurement of RNA decay using molecular lanterns. Top: an RNA with three stem-loop structures assembles an active luciferase, which lights up the nearby YFP via BRET. Exonucleolytic digestion from the 3′ terminus (Pacman) leads to digestion of the weak Box-B structure, but pauses at the B′ helix (bottom), leading to increased bioluminescence.

FIG. 25 shows a non-limiting example for visualizing polyadenylation dynamics with molecular lanterns. Circular RNAs (circRNA) targeting polyA tails on mRNA transcript will be used to dimerize split luciferase MS2/PP7.

FIGS. 26A and 26B shows bioluminescence-based tracking of translated mRNAs. FIG. 26A shows mRNA will code for protein of interest (e.g. Arc) fused to N-luc and MS2, which will bind its cognate stem-loop at the end of the ORF (A). The second stem-loop will bind PP7-C-luc, yielding active luciferase when the mRNA is translated. FIG. 26B shows in the second system, co-transcriptionally, the MS2-binding stem-loop will not form, because an alternative structure (A-A) will sequester part of it (A). A translating ribosome will unfold the structure, allowing the MS2-N-luc to bind and yield photons.

FIG. 27 shows aptamer-mediated dimerization of split luciferase proteins. N-Fluc and C-Fluc, fragments show no catalytic activity alone; when dimerized by an aptamer, an active luciferase is formed, vastly enhancing photon production.

### DETAILED DESCRIPTION OF THE INVENTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

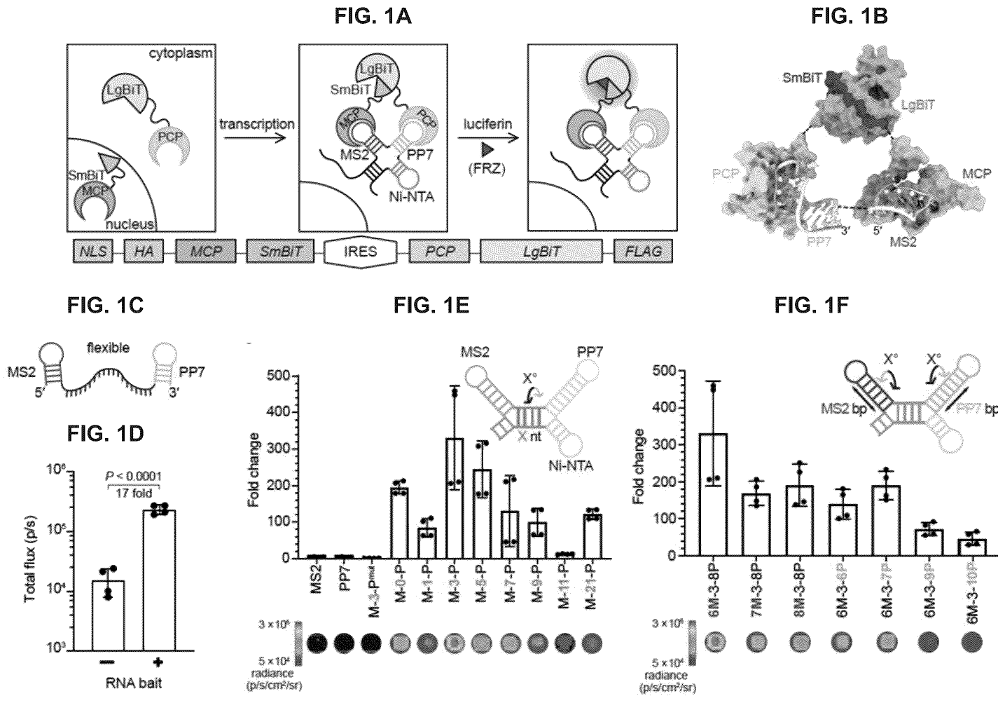

Referring now to FIGS. 1-27, the present invention features compositions (e.g., RNA lanterns) and methods to visualize RNA dynamics.

The present invention features a bioluminescent composition (e.g., RNA lanterns) for visualizing RNA dynamics. In some embodiments, the composition comprises an RNA (e.g., a structured RNA) comprising at least a first stem-loop, wherein the first stem-loop is (or may be) bound by a first RNA-interacting protein linked to a first portion of a detectable reporter (e.g., a first portion of a first detectable reporter). In other embodiments, the composition comprises an RNA comprising a first stem-loop that is (or may be) bound by a first RNA-interacting protein linked to a first portion of a detectable reporter (e.g., a first portion of a first detectable reporter) and a second stem-loop that is (or may be) bound by a second RNA-interacting protein linked to a second portion of the detectable reporter (e.g., a second portion of the first detectable reporter). In further embodiments, the composition comprises an RNA comprising a first stem-loop that is (or may be) bound by a first RNA-interacting protein linked to a first portion of a detectable reporter (e.g., a first portion of a first detectable reporter), a second stem-loop that is (or may be) bound by a second RNA-interacting protein linked to a second portion of the detectable reporter (e.g., a second portion of the first detectable reporter), and a third stem-loop that is (or may be) bound to a second detectable reporter. In some embodiments, the third stem-loop is downstream of the first stem-loop and/or the second stem-loop.

In some embodiments, the detectable reporter (e.g., the first or second detectable reporter) comprises an optical reporter. In some embodiments, the detectable reporter (e.g., the first or second detectable reporter) comprises a fluorescent reporter. Non-limiting examples of fluorescent reporters include yellow fluorescent proteins (YFP), red fluorescent proteins (RFP), or green fluorescent proteins (GFP). In some embodiments, the detectable reporter (e.g., the first or second detectable reporter) comprises a light-emitting luciferase protein. In some embodiments, the detectable reporter (e.g., the first or second detectable reporter) comprises a proximity labeling protein. The present invention is not limited to the aforementioned detectable reporters and may include any suitable reporters for use in the compositions and methods described herein. In some embodiments, the present invention encompasses any imaging modality for which split genetic reporters are available, including but not limited to fluorescent proteins for fluorescence imaging, split reporters for MRI, PET, SPECT, and similar technologies.

In some embodiments, the first portion of the detectable reporter comprises a first portion of a light-emitting luciferase protein (e.g., the N-terminal fragment of the light-emitting luciferase protein). In some embodiments, an MS2 RNA binding protein (e.g., the first RNA-interacting protein) is linked to the N-terminal fragment of the light-emitting luciferase protein. In certain embodiments, the MS2 RNA binding protein is linked to the N-terminal fragment via a linker.

In some embodiments, the second portion of the detectable reporter comprises a second portion of the light-emitting luciferase protein (e.g., the C-terminal fragment of the light-emitting luciferase protein). In some embodiments, the PP7 RNA binding protein (e.g., the second RNA-interacting protein) is linked to the C-terminal fragment of the light-emitting luciferase protein. In certain embodiments, the PP7 RNA binding protein is linked to the C-terminal fragment via a linker.

In some embodiments, the detectable reporter becomes functional when the first portion of the detectable reporter and the second portion of the detectable reporter are brought together. In other embodiments, the detectable reporter comprises a light-emitting luciferase protein, wherein the light emitting luciferase protein becomes functional when the first portion and the second portion are brought together

In some embodiments, the linker is short (e.g. one to about 5 amino acids in length), hydrophobic, or a combination thereof.

In some embodiments, the RNA-interacting protein (e.g., the first RNA-interacting protein or the second RNA-interacting protein) comprises an MS2 RNA-binding protein, a PP7 RNA binding protein, an L7Ae RNA binding protein, or a lambdaN RNA binding protein. Moreover, the present invention extends beyond the aforementioned RNA-interacting proteins and encompasses additional RNA-interacting proteins, along with their evolved variants.

In some embodiments, the 5′ end of the RNA and the 3′ end of the RNA are complementary and form an RNA stem. In certain embodiments, the first stem-loop and the second stem-loop are between the complementary 5′ end and the 3′ end of the RNA. In other embodiments, the 5′ end of the RNA and the 3′ end of the RNA are non-complementary and form single-stranded RNA (ssRNA) arms. In some embodiments, the ssRNA arms flank the first stem-loop and the second stem-loop. Without wishing to limit the present invention to any theory or mechanism, it is believed that the use of complementary 5′ and 3′ ends of an RNA provides different rigidified structures.

In some embodiments, the composition comprises a structured RNA. As used here, a “structured RNA” refers to an RNA comprising extensive base pairing, tertiary interactions, and potentially quaternary interactions, or a combination thereof. In certain embodiments, the composition comprises an unstructured RNA until a target RNA base pairs with the ssRNA arms, such that when the target RNA binds to the ssRNA arms, a structured RNA is formed. In certain embodiments, two stem-loops are not connected by flexible linkers; rather, the overall bioluminescent composition comprises a 3-way or 4-way junction, rendering it rigidified. In other embodiments, the binding sites of the proteins may be on the same helical segment interrupted by loops that form the protein-binding structures, for example, an RNA domain that contains a kink-turn structure recognized by the L7ae protein is fused to a stem-loop that binds the lambdaN protein. Such a domain would be considered a rigid single stem-bulge-loop RNA structure that can assemble two distinct RNA-binding proteins.

In some embodiments, the target RNA is a cellular RNA. In some embodiments, the cellular RNA comprises a messenger RNA (mRNA), a splice variant of an mRNA, a microRNA, or a non-coding RNA (ncRNA).

In some embodiments, the composition comprises a structured RNA formed by trans-splicing, RNA editing, or genome editing. For example, a 3′ untranslated region of an mRNA may be edited using a group I self-splicing intron modified to recognize the target RNA, perform a splicing reaction in trans to exchange the endogenous RNA with an introduced RNA that will form the structured bioluminescent complex. The formed bioluminescent composition (e.g., RNA lantern) will comprise sequences from the original RNA base-paired with the introduced RNA sequence, such that a rigid RNA structure is formed. The trans-splicing or editing reaction may include downstream sequences that faithfully copy the endogenous mRNA sequences, including polyadenylated tails or introduce components from other RNAs or both.

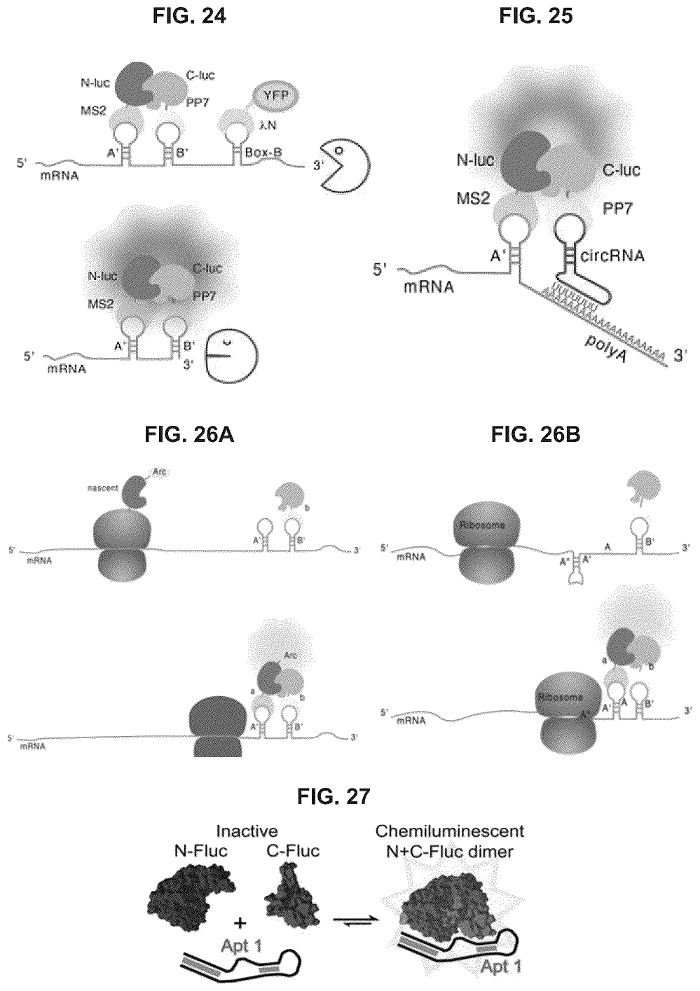

Referring to FIG. 24, the present invention may feature a bioluminescent composition (RNA lantern) for visualizing RNA decay. For example, the composition may comprise an RNA comprising a first stem-loop bound by a first RNA-interacting protein linked to a first portion of a detectable reporter, a second stem-loop bound by a second RNA-interacting protein linked to a second portion of the detectable reporter, and a third stem-loop bound to a second detectable reporter and downstream the first stem-loop and the second stem-loop. For example, The first and the second stem-loop may be bound by RNA-interacting proteins (e.g., MS2 and PP7, respectively) encoding a split luciferase. The two stem-loops will be closely followed by a third, weak Box-B stem-loop that binds the λN protein fused to YFP (FIG. 24). When all three stem-loops are present in the 3′ UTR, the BRET signal will be yellow. A decaying mRNA, undergoing digestion from the 3′ terminus by an exonuclease, will lose the third stem-loop and the BRET partner of the luciferase. The luminescence of the partially decayed mRNA will thus increase, be differentially colored, and stable due to the stability of the first two stem-loop structures. Thus, in some embodiments, the RNA may undergo 3′ RNA decay. For example, when the RNA undergoes 3′ RNA decay by a 3′ exonuclease, the third stem-loop will be digested by the 3′ exonuclease and detectable reporter (e.g., the light-emitting luciferase protein) will be becomes functional, e.g., the luminescences from the functional light emitting luciferase protein will be observed.

Referring to FIG. 25, the present invention may feature a bioluminescent composition (RNA lantern) for visualizing RNA polyadenylation. In some embodiments, the first stem-loop may be at or near the 3′ end of the RNA. In certain embodiments, the composition comprises an RNA comprising a first stem-loop at or near the 3′ end of the RNA, wherein the first stem-loop is bound by a first RNA-interacting protein linked to a first portion of a detectable reporter and a circular RNA comprising a stem-loop (e.g., a second stem-loop) and a polyU sequence. The polyU sequence may bind to at least a portion of a polyA sequence of the RNA (e.g., the RNA comprising the first stem-loop). In some embodiments, the stem-loop (e.g., the second stem-loop) is bound by a second RNA-interacting protein linked to a second portion of a detectable reporter.

Referring to FIGS. 26A and 26B, the present invention may feature a bioluminescent composition (RNA lantern) for visualizing RNA translation (e.g., nascent translation and on-going translation). In some embodiments, the composition may comprise an RNA comprising a first stem-loop and a second stem-loop, both at or near the 3′ end (e.g., in the 3′ UTR at a location near the end of the coding region) of the RNA. In some embodiments, the first stem-loop is bound by a first RNA-interacting protein linked to a first portion of a detectable reporter. In some embodiments, the second portion of the detectable reporter may be expressed independently. In some embodiments, when the first portion of the detectable reporter and the second portion of the detectable reporter are brought together, the detectable reporter will be functional (e.g., when luciferase pairs are brought together, the luciferase will produce photons). When the ribosome dissociates, the nascent polypeptide will remain bound to the MS2-binding stem-loop and continue to glow, providing a time stamp for the first round of translation (FIG. 26A).

In some embodiments, the composition may comprise an RNA comprising a first stem-loop and a second stem-loop, both at or near the 3′ end (e.g., in the 3′ UTR at a location near the end of the coding region) of the RNA. However, in certain embodiments, the first stem-loop will comprise a conformational switch into the 3′ terminus of the coding sequence (FIG. 26B), similar to bacterial transcriptional termination-anti-termination helices. A key property of these switches is that the RNA structure that forms co-transcriptionally is kinetically favored (kinetically trapped), but thermodynamically less stable than the alternative structure that binds a RNA-interacting protein (e.g., MS2). A translating ribosome will unwind the first structure, allowing its second half to form a stem-loop of the MS2-binding site. The stop codon will be placed such that the ribosome will not unwind the MS2-binding stem-loop. In some embodiments, the untranslated mRNA will produce no photons, but an actively translating ribosome will switch the mRNA into a luminescent lantern.

The aforementioned bioluminescent composition for visualizing RNA translation is not limited to having an RNA with a first stem-loop and a second stem-loop both at or near the 3′ end. In some embodiments, the composition may comprise an RNA comprising a first stem-loop and a second stem-loop, both at or near the 5′ end of the RNA. Additionally, in certain embodiments, the composition may comprise an RNA with a first stem-loop and a second stem-loop located at any position within the RNA. In certain embodiments, the bioluminescent composition (RNA lantern) described herein may be formed as a product of splicing or other RNA processing, affording the study of such RNA processing in live cells and animals.

In some embodiments, the bioluminescent composition described herein may be used to visualize the life cycle of an RNA (e.g., polyadenylation, translation, and degradation), e.g., in live animals. In some embodiments, AAV vectors may be used to introduce the luminescent constructs and their target mRNAs into an animal, e.g., neurons in live mice. For mRNAs that contain the MS2, PP7, and Box-B target stem-loops, the genes will be prepared with inducible promoters to allow synchronization of transcription initiation. Validation and control constructs use strong constitutive promoters, such as CMV, to promote RNA production and benchmark detection.

Referring to FIG. 27, in some embodiments, the present invention features a composition comprising a single RNA element that can be directly fused to the RNA targets. In some embodiments, the composition may utilize RNA aptamer or ribozymes, instead of enzyme fusions to generate an output (e.g., photons).

### Example 1

The following is a non-limiting example of the present invention. It is to be understood that said example is not intended to limit the present invention in any way. Equivalents or substitutes are within the scope of the present invention.

General strategy for visualizing RNAs with bioluminescent light: The overall strategy was dependent on three key steps: RNA bait formation, MCP/PCP binding, and NanoBiT complementation. Each part of a NanoBiT system (e.g., the short peptide—SmBiT—and the larger protein fragment-LgBiT, FIG. 1A) was appended to MCP and PCP, respectively. The resulting fusions (termed RNA lanterns) bind to their cognate aptamers on a single transcript, inducing NanoBiT complementation and generating photons in the presence of luciferin. The requisite components had not been used together prior to this work, necessitating optimization of each step.

As a starting point, a bicistronic construct was designed with an internal ribosome entry site (IRES) to mediate co-expression of the RNA lantern components (FIG. 1A). MCP was fused to the short peptide fragment (SmBiT), and PCP was fused to the larger protein fragment (LgBiT). MCP was further tagged with a nuclear localization signal (NLS) to reduce background complementation. Upon expression, transcripts fused to the RNA bait could transport an MCP-fusion from the nucleus into the cytoplasm (or capture de novo translated MCP in the cytoplasm). Eventual binding of the PCP fusion would co-localize both halves of the RNA lantern on the target transcript, enabling NanoBiT complementation and, thus, light production. Additionally, the NLS reduces the likelihood of non-specific SmBiT/LgBiT complementation, a problem encountered in previous studies that resulted in diminished sensitivity.

The RNA lanterns were modeled using ChimeraX20 to assess the design of the fusions (FIG. 1B). No obvious steric clashes or unfavorable orientations with a 5′-MS2-PP7-3′ RNA bait were observed, suggesting that lantern assembly was possible. NanoBiT complementation appeared feasible even with juxtaposed MS2 and PP7 aptamers. Small, compact baits are attractive tags to avoid disrupting the structures or functions of target RNAs. Encouraged by the modeling results, this lantern design was advanced with the anticipation that additional engineering of the lantern components (linkers and orientation) would be necessary to maximize luciferase complementation and signal output.

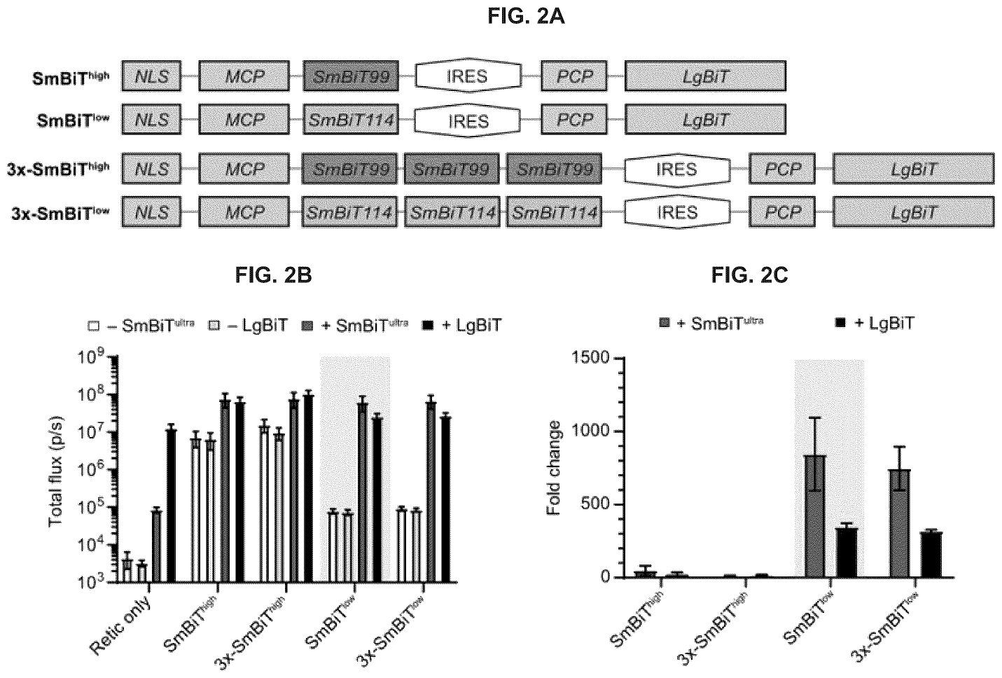

Biochemical optimization of the RNA tag and lanterns: The RNA detection platform relies on efficient NanoBiT formation, which can be tuned based on the SmBiT peptide sequence. Two SmBiT peptides (SmBiT^{high}, KD=180 nM; SmBiT^{low}, KD=190 μM) were examined to determine which would provide the best dynamic range: minimal signal in the absence of RNA bait and robust signal in its presence (FIG. 2A). The designer probes were first expressed using an in vitro transcription/translation system (IVTT) featuring transcription by T7 RNA polymerase and translation in rabbit reticulocyte lysate (RRL). Background signal was determined in the absence of RNA bait. Full complementation of translated NanoBiT was achieved by adding exogenous SmBiT^{ultra} (KD=0.7 nM) or recombinant LgBiT at saturating concentrations, establishing the maximum potential signal (FIG. 2B). All protein designs exhibited robust signal enhancement when SmBiT^{ultra} or recombinant LgBiT was added, but high background luminescence was observed in samples with SmBiT^{high}. The largest signal enhancements were achieved with SmBiT^{low}, due to the reduced background complementation observed with this peptide (FIG. 2C). Additional repeats of SmBiT did not yield greater light output, possibly due to insufficient spacing between sequential peptides to support LgBiT binding. Therefore, MCP-SmBiT^{low}/PCP-LgBiT was moved forward with the lantern combination that provided the highest dynamic range.

Whether the MCP-SmBiT^{low}/PCP-LgBiT lantern could detect the previously reported MS2-PP7 bait, comprising an unstructured 19-nucleotide strand joining the two aptamers was determined (FIG. 1C). Upon RNA bait transcription, an approximate 17-fold signal increase was observed (FIG. 1D). The relatively low signal enhancement was attributed to the RNA bait structure. Secondary structure modeling with Vienna RNAfold and Forna, revealed that the 19-nucleotide linker was likely flexible with the potential to sample non-productive conformations. While an unstructured RNA bait could aid lantern binding, extensive flexibility could potentially hinder NanoBiT complementation and thus diminish signal-to-noise ratios.

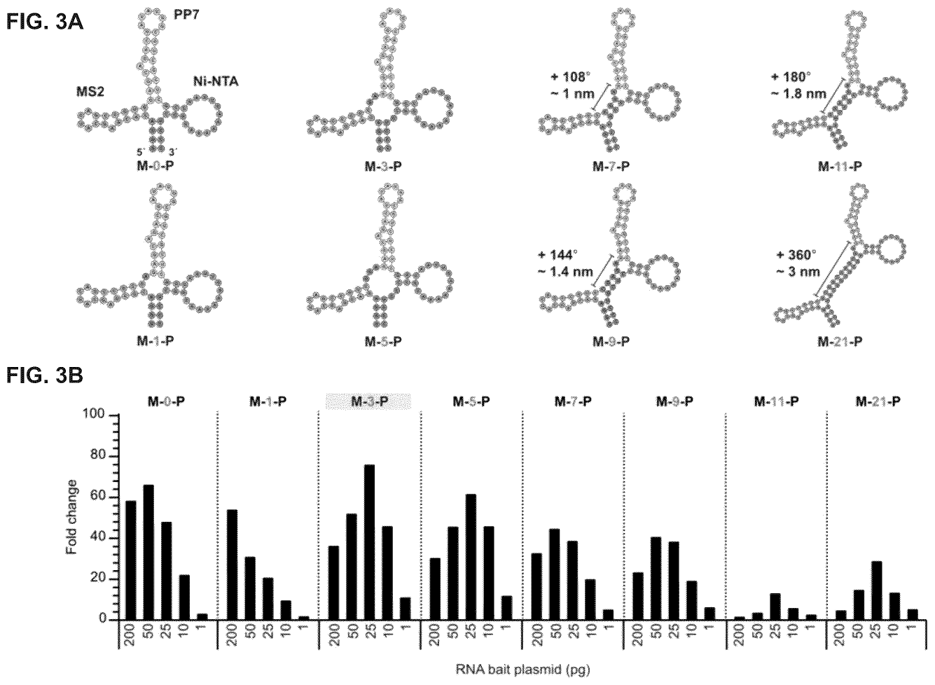

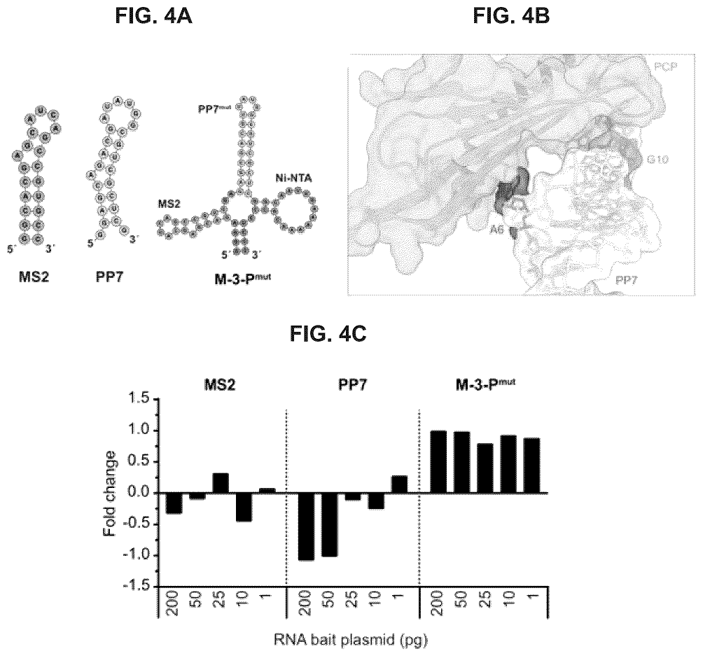

Thus, increasing the rigidity of the RNA bait may favor lantern assembly and photon production. Therefore, the RNA bait was redesigned to fix the spacing and orientation of the MS2 and PP7 sequences. The aptamers were locked into desired conformations, as part of a four-way junction with an established Ni-NTA-binding aptamer (FIG. 1E and FIG. 3A). The RNA baits were further engineered to contain varying numbers of nucleotides between the MS2 and PP7 aptamer domains (M-X-P; where X is the number of nucleotides in the spacer). This panel facilitated the sampling of not only the spacing between the aptamer domains (˜3 nm, according to secondary structure predictions) but also the helical phase of the aptamers respective to one another (up to ˜360°). When the RNAs were present with the lantern fragments, a relatively short linker (M-3-P) could yield significantly higher luminescence, with up to a 330-fold increase over a no-RNA control (FIG. 1E). In the case of M-11-P, the signal was abolished. This result was attributed to the phase angle of approximately 180° (one half of a helical turn) from M-3-P, preventing luciferase complementation. Importantly, signal was restored with the MS2 and PP7 aptamers brought back into phase (˜360°) and a full helical turn apart (M-21-P). Signal still decreased because the overall length increased and likely prevented effective assembly of the luciferase parts. Photon production was also highly dependent on the concentrations of the RNA bait and the RNA lantern (FIG. 3B) and RNA bait integrity. In experiments using singular MS2 and PP7 aptamers, or M-3-P^{mut} that does not bind the PCP protein (FIG. 4), no appreciable signal over background was observed (FIG. 1E and FIG. 4C).

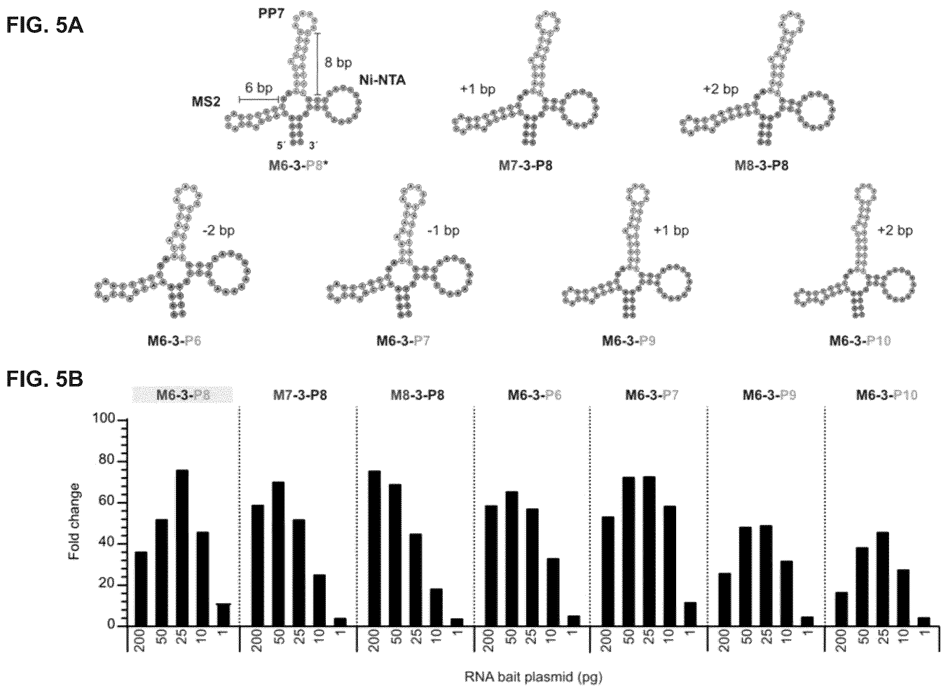

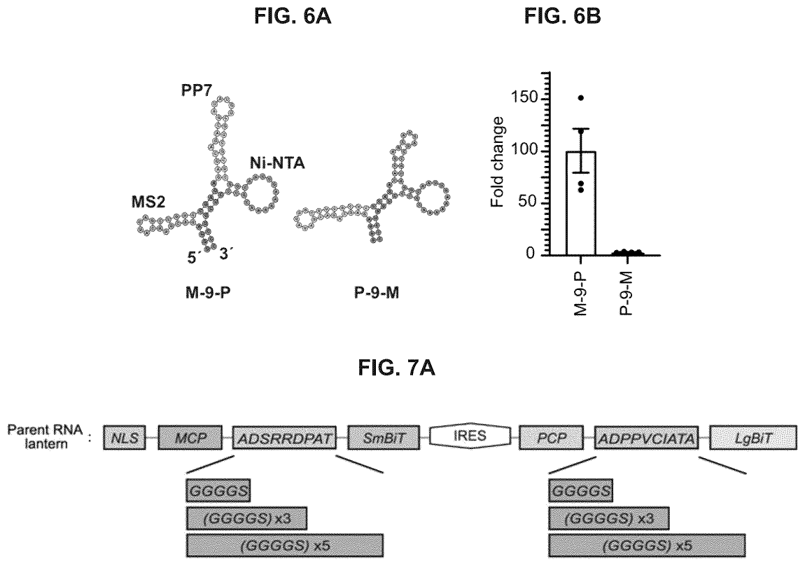

Due to the improved luminescence achieved through modulating the distance and phase angle, the length and relative orientation of the individual MS2 and PP7 aptamers was also examined. The optimal M-3-P RNA bait was adjusted by adding or removing base-pairs (bp) to either the MS2 or PP7 stem. (FIG. 1F and FIG. 5A). No significant improvements in photon output were observed among the suite of RNA baits, suggesting that the length and orientation of the two aptamers were already optimal. Additionally, the lack of improvement was not a result of template DNA ratios (FIG. 5B). Although the orientation of the aptamers had minimal effect on complementation, the positioning of the aptamers was critical. When the placement of MS2 and PP7 were inverted, luciferase complementation was not observed (FIG. 6).

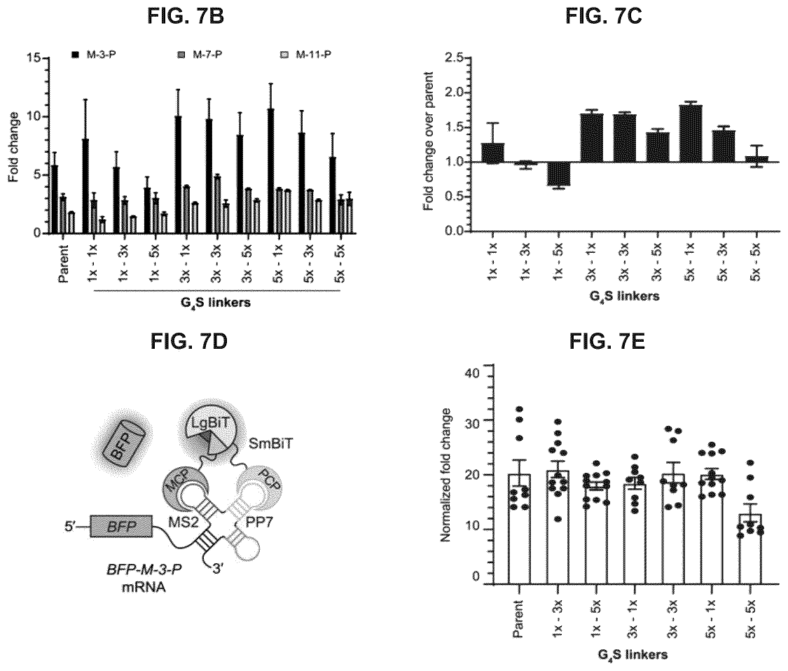

The effect of protein linker length on RNA lantern assembly was further examined. Constructs were designed with additional glycine-serine (G4S) units inserted between the RNA-binding proteins and NanoBiT segments (FIG. 7A). These constructs were then tested with the M-3-P, M-7-P, and M-11-P RNA baits (FIG. 7B). The largest light outputs were achieved with the M-3-P RNA bait, for all the lanterns tested. Interestingly, only minimal signal enhancements (˜1.5 fold) were observed with the additional G4S units compared to the original lanterns both in vitro and in cells (FIGS. 7C, 7D, and 7E).

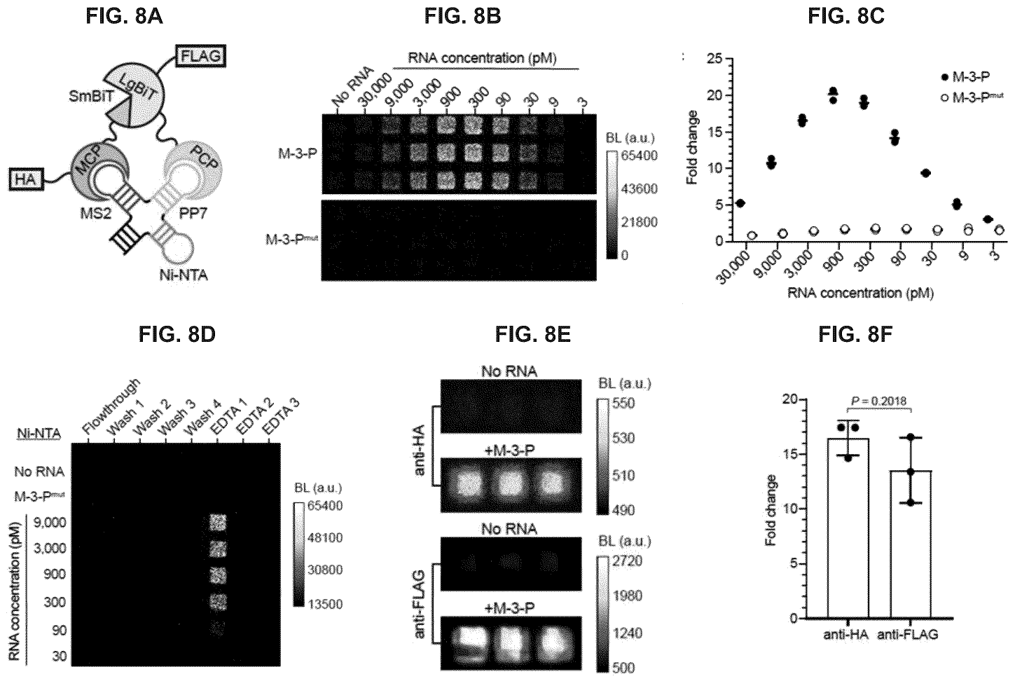

The RNA sensing capabilities of the designer bait and lanterns were biochemically validated in vitro (FIG. 8A). Cells were engineered to stably express the lantern components, and lysates were then titrated with purified bait. An RNA-dependent “hook effect” was observed (FIGS. 8B and 8C). Lantern complex formation was limited at low RNA concentrations. More lantern complementation was observed with increasing RNA bait, but the amount falls off at high RNA concentrations. This latter outcome is explained by MCP and PCP proteins binding distinct RNA bait transcripts, so that the luciferase bits are not brought together to assemble active lantern complexes.

The unique design of the RNA bait enabled direct interrogation of lantern binding and assembly. The Ni-NTA aptamer was used to retrieve the RNA bait-RNA lantern complex on resin: as shown in FIG. 8D, the assembled NanoBiT enzyme was exclusively associated with the RNA bait (FIG. 2D). Pulldowns of active complexes using the HA and FLAG tags built into the RNA lantern components further confirmed the RNA-dependent assembly of the active luciferase (FIGS. 8E and 8F). The retrieval of targeted transcripts and associated biomolecules post-imaging will facilitate the often-critical follow-up analyses of RNA interactions.

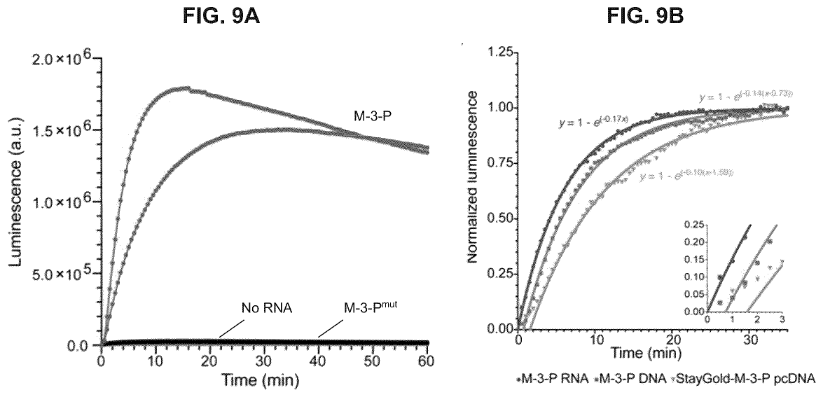

Real-time imaging of RNAs in cells demands fast signal turn-on in the presence of target transcripts. Thus, the kinetics of lantern assembly were evaluated in vitro. M-3-P bait was first added to lysate from lantern-expressing cells, and luminescence measurements were collected over time (FIG. 9A). Signal was immediately detected upon RNA addition, with peak luminescence reached within 20 min. No luminescence was observed in the absence of RNA or when control M-3-P^{mut} bait was added to the lysate. The lanterns can also report on gene expression in real-time. The split luciferase fragments were expressed in reticulocyte lysate prior to addition of either purified M-3-P RNA bait, M-3-P DNA, or DNA encoding GFP-M-3-P (FIG. 9B). As expected, the signal was immediately observed from samples containing the RNA bait. The onset of luminescence was delayed, though, for the samples comprising DNA bait, owing to the need for target transcription prior to lantern binding. The longest delay was observed with GFP-M-3-P, the construct with the largest sequence upstream of M-3-P. Collectively, these data suggested that the RNA lantern and rigidified tags can provide reliable readouts on RNA dynamics.

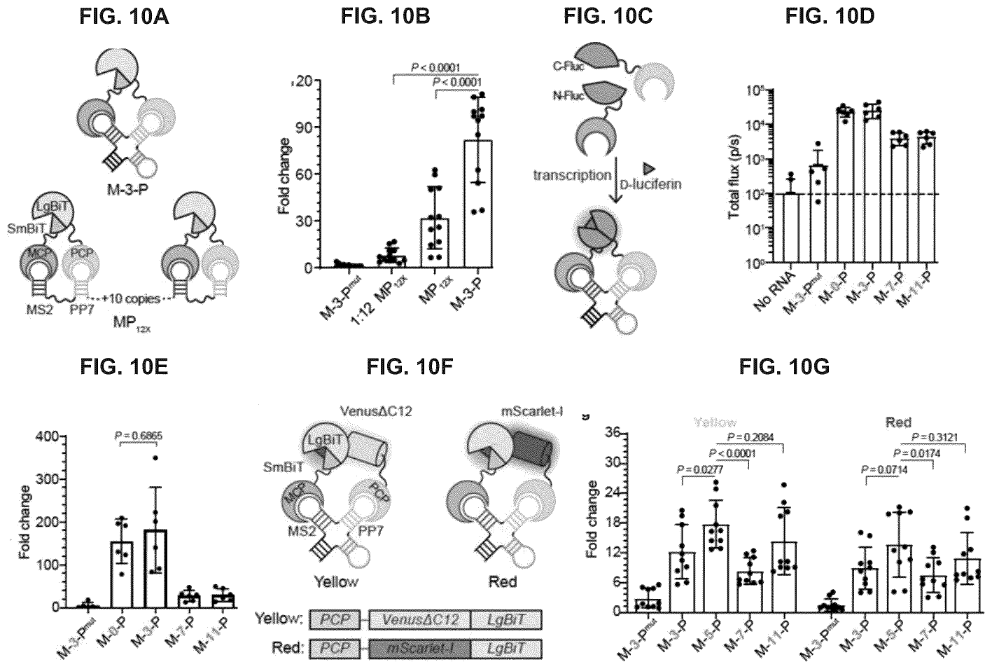

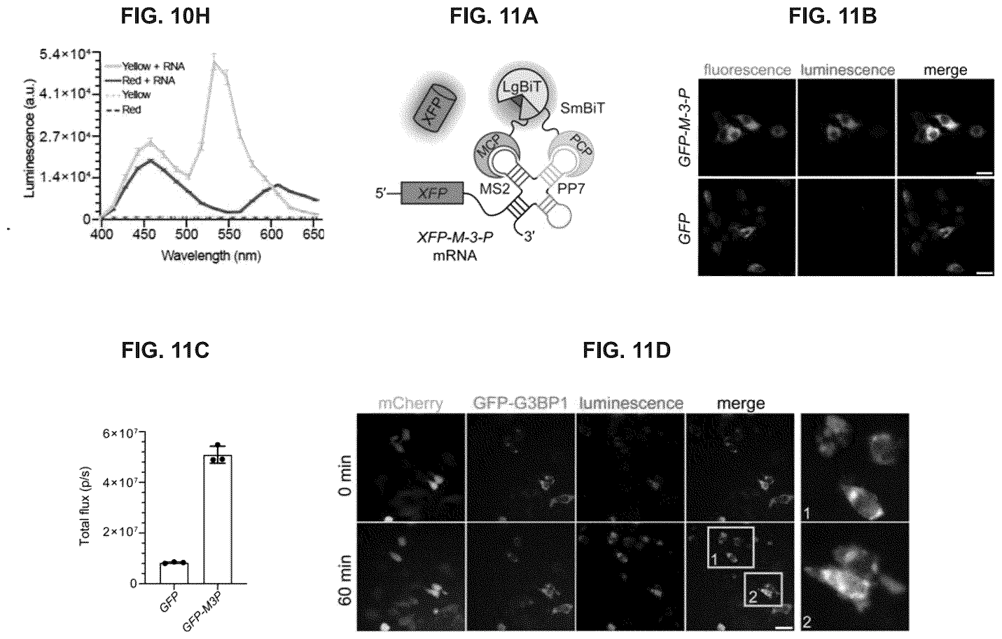

RNA imaging platform is ultrasensitive and modular: Previous applications of MS2-PP7 for fluorescence imaging have required at least 12 copies of the aptamers to achieve adequate signal-to noise ratios (MP12X, FIG. 10A). In many cases, these constructs comprise an RNA bait that is at least 780 nucleotides long, a size that may impede the natural localization and behavior of the RNA under study. The M-3-P bait (69 nucleotides) requires only a single copy of each aptamer to produce a detectable signal (FIG. 10B). When compared head-to-head with bait aptamers at equimolar concentration (M-3-P versus one twelfth RNA concentration of MP12X), the structured bait produced 10-times more signal. Even when MP12X was introduced at the same RNA concentration and the aptamers were 12-times more concentrated, the structured M-3-P bait outperformed the flexible design (FIG. 10B). These results suggest that the compact M-3-P is a more effective RNA bait, providing higher signal compared to the multimerized and extensively used MS2-PP7 tag.

The designer M-3-P bait can also facilitate complementation of other split reporters. When MCP and PCP were fused to split fragments of firefly luciferase (Fluc; FIG. 10C), the resulting RNA lantern yielded a large increase in signal over background (FIGS. 10D and 10E). The photon flux of the assembled Fluc is lower than NanoBiT, but the undetectable background in absence of RNA may be desirable in specific applications. M-3-P also enabled the assembly of bioluminescence resonance energy transfer (BRET) variants of NanoLuc. These probes comprise luciferase fluorescent protein fusions that produce red-shifted light upon complementation. Such wavelengths can provide more sensitive readouts in vivo because they are less absorbed and scattered by tissue. Two BRET-based RNA lanterns were developed using yellow (VenusΔC12) 9 and red (mScarlet-1) fluorescent proteins fused to the C-terminus of PP7 and the N-terminus of LgBiT (FIG. 10F). Assembly of the yellow and red RNA lanterns was assessed by measuring photon production in the presence of rigidified RNA baits. M-5-P provided the highest degree of signal turn-on (FIG. 10G). The larger bait provides more space between the bulkier lantern fragments in these cases, likely enabling more effective complementation. The BRET-bases probes also exhibited different colors of light output (FIG. 10H). BRET efficiency was higher for the yellow than the red lantern due to the larger spectral overlap between NanoBiT and VenusΔC12 compared to mScarlet-1. Collectively, these demonstrate that structured RNA baits can productively assemble diverse split proteins.

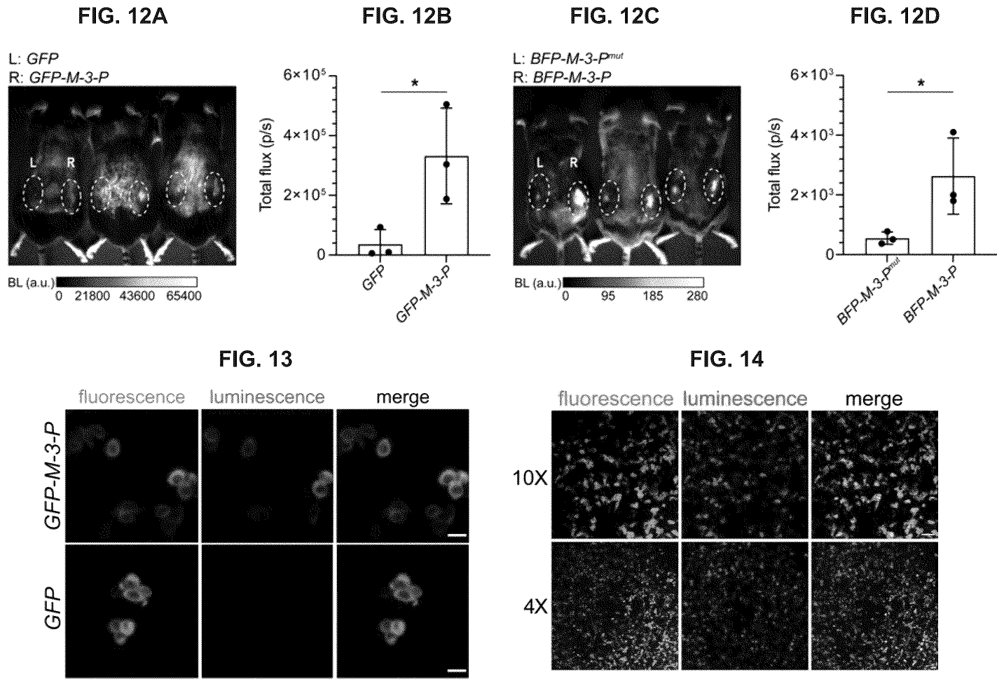

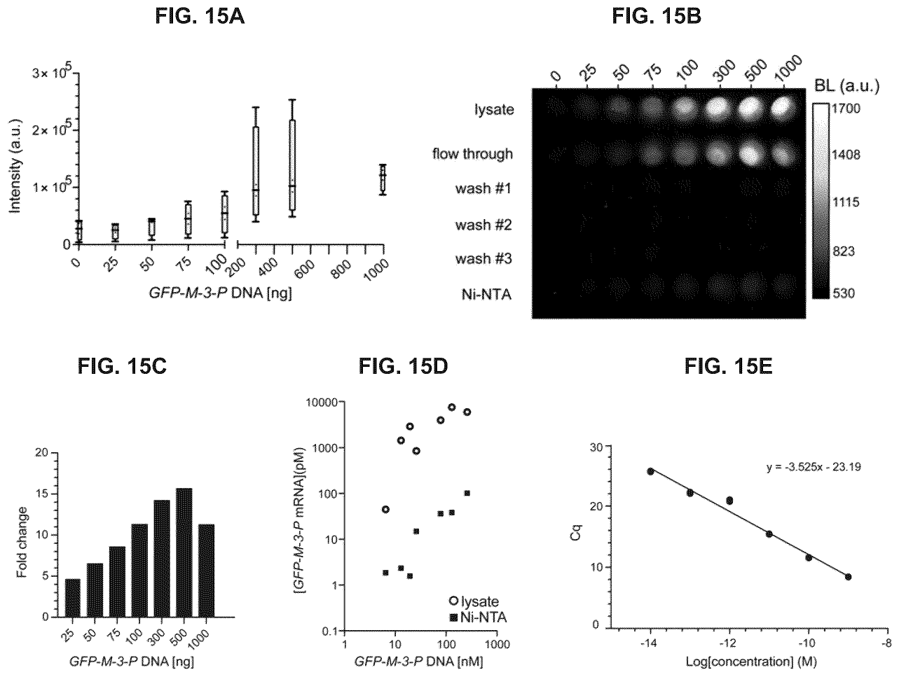

Imaging RNAs from the micro-to-macro scale: The robustness of the bait design enabled facile imaging of RNA both in cellulo and in vivo. As an initial test, a model system was developed using an mRNA encoding green fluorescent protein (GFP) with the M-3-P RNA bait placed in the 3′ untranslated region (UTR) (FIG. 11A). GFP fluorescence would thus report on bait expression. Cell lines stably expressing RNA lanterns were generated and transfected with constructs encoding M-3-P-tagged GFP or GFP only. GFP fluorescence was observed in both cases, but bioluminescence (from RNA lantern assembly) was only observed in cells transfected with M-3-P-tagged GFP (FIGS. 11B and 11C, and FIG. 13, FIG. 14, and FIG. 15A). Similar signal turn-on was observed using an analogous model transcript (FIGS. 12D and 12E). The number of assembled lanterns in the cells was further quantified via pulldown assays. RNA bait-RNA lantern complexes were retrieved via the Ni-NTA aptamer as before (FIG. 15B). The amount of captured complex (assessed via luminescence measurements) correlated with the amount of target DNA and downstream mRNA produced (FIGS. 15C, 15D, and 15E)

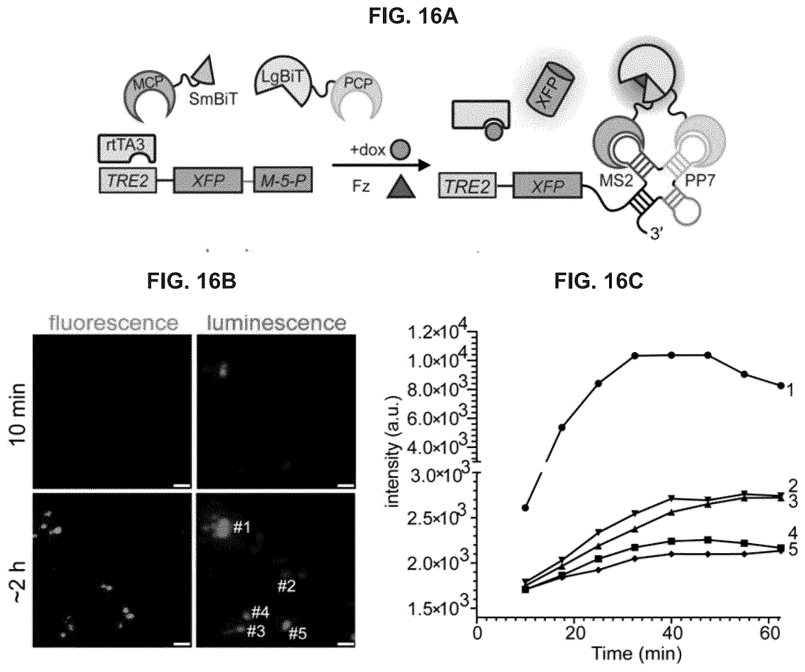

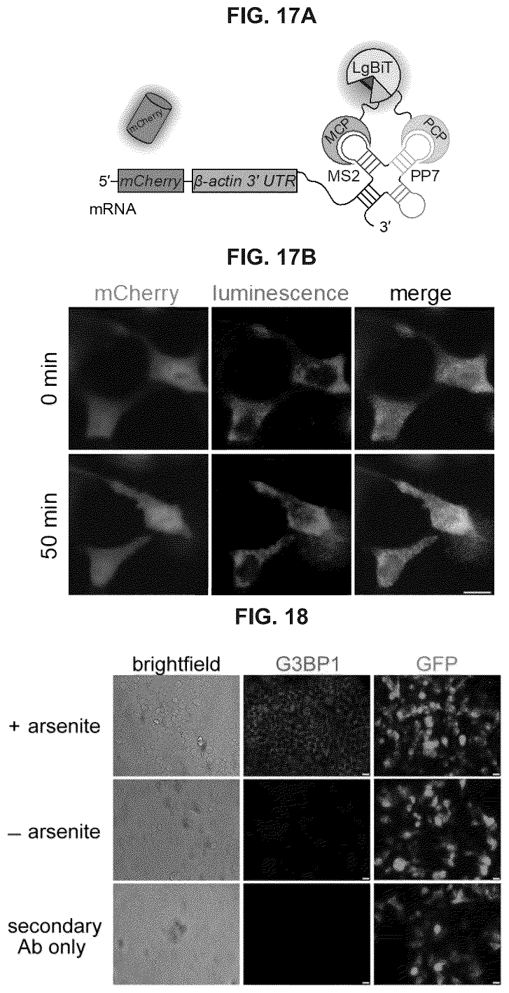

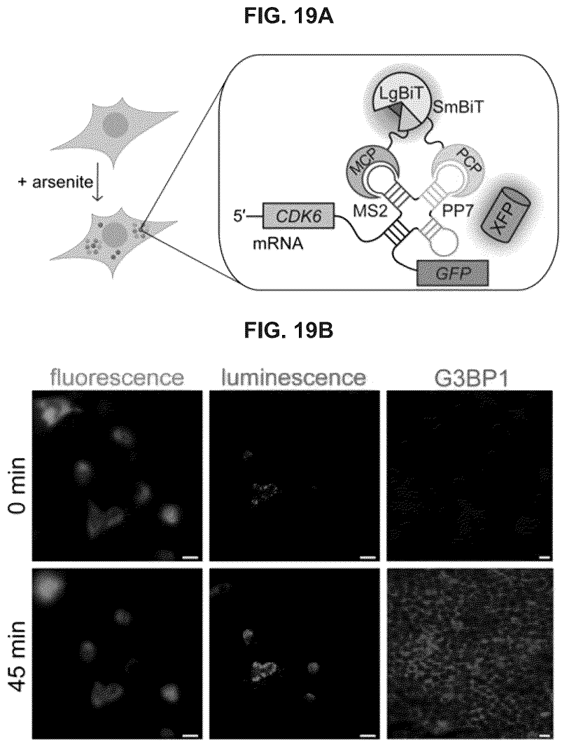

The generality of the RNA lanterns and tags for imaging model transcripts was further established. An inducible expression system to monitor the production of a fluorescent protein transcript tagged with M-5-P was first evaluated (FIG. 16A). Luminescence turn-on was observed in the first 10 min of induction, with fluorescent signal (following subsequent protein translation and chromophore maturation) appearing later (FIGS. 16B and 16C). The lanterns were also used to dynamically trace biologically relevant targets, including beta-actin RNA (FIG. 17A). This transcript is known to localize to stress granules upon arsenite treatment. The lantern and tag set shown in FIG. 11A-11D was used to monitor the RNA over time. Transcript localization to stress granules was observed, corroborating previous reports (FIG. 11D, FIG. 17B, and FIG. 18). Similar analyses were performed with other model transcript, CDK6 (FIGS. 19A and 19B). Collectively, these studies illustrate the utility of the structured bait for RNA lantern assembly and transcript visualization.

Finally, as a proof of concept, RNAs were imaged in subcutaneous models in vivo. HEK293T cells were engineered to express RNA lanterns and mRNAs encoding either GFP or BFP with variable 3′ UTRs: M-3-P, M-3-P^{mut}, or no bait (FIG. 10A). The cells were incubated with luciferin, implanted in mice dorsal flanks, and imaged. Increased luminescence was observed from cells expressing RNA lanterns and GFP-M-3-P (right flank) compared to cells lacking the M-3-P bait (left flank (FIGS. 13A and 13B). Similar increases in luminescence were observed from cells expressing BFP-M-3-P, compared to control transcripts in the presence of the lantern components (FIGS. 12C and 12D). These data demonstrate that lanterns can be used for RNA-dependent imaging in tissue, setting the stage for real-time detection of gene expression in live animals.

RNA dynamics have been historically visualized in living systems with fluorescent probes. Such methods require optically permissive platforms such as transparent model organisms or surgically implanted windows. The required excitation light can further induce high background signals in tissue and is often limited in depth. Bioluminescent probes (luciferases) overcome some of these limitations. Luciferase reporters use enzymatic reactions—instead of excitation light—to generate photons, which produces far less background signal and can be advantageous for serial imaging in tissue and whole animals.

To capitalize on the sensitivity and dynamic range of bioluminescence, the present invention features a genetically encoded split luciferase-based platform for visualizing RNA transcripts. The RNA lanterns combine the well-known NanoBiT system and MS2/PP7 platform for transcript tracking. The components were systematically optimized to achieve sensitive imaging. Substantial improvements in signal production were achieved by modulating the spacing and phase angle of the aptamer components. The optimized design significantly decreases the size of previously reported RNA-protein complexes for imaging. Only a single copy of the RNA bait was necessary for imaging target transcripts in cells and whole organisms.

The RNA bait and lanterns also comprise unique features for a range of applications. Both the proteins and bait are equipped with affinity tags for retrieval and downstream analyses of target transcripts and interacting biomolecules. Structured RNA baits can productively assemble diverse split proteins, including Fluc and various BRET reporters. Such modularity expands the color palette of RNA lanterns and sets the stage for even deeper tissue imaging and multiplexed assays. The BRET probes were more efficiently assembled with longer RNA baits, though care should be taken to optimize each lantern and tag combination.

RNA lanterns enable RNA dynamics to be visualized in vitro and in vivo. The probes will be particularly useful for serial imaging of transcripts, where localization and expression are difficult to examine over time.

General information: Q5 DNA polymerase, restriction enzymes, and all buffers were purchased from New England Biolabs. dNTPs were purchased from Thermo Fisher Scientific. Luria-Bertani medium (LB) was purchased from Genesee Scientific. All plasmids and primer stocks were stored at −20° C. unless otherwise noted. Primers were purchased from Integrated DNA Technologies and plasmids were sequenced by Azenta Life Sciences. Sequencing traces were analyzed using Benchling.

General cloning methods: Polymerase chain reaction (PCR) was used to prepare genes of interest, and products were analyzed by gel electrophoresis. Products were excised and purified. Amplified genes were ligated into destination vectors via Gibson assembly34. All PCR reactions were performed in a BioRad C3000 thermocycler using the following conditions: 1) 95° C. for 3 min, 2) 95° C. for 30 s, 3)—1.2° C. per cycle starting at 72° C. for 30 s, 4) 72° C. for 30 sec, repeat steps 2-4 ten times, 5) 95° C. for 3 min, 6) 95° C. for 30 s, 7) 60° C. for 30 s, 8) 72° C. for 2 min repeat steps 6-8 twenty times, then 72° C. for 5 min, and hold at 12° C. until retrieval from the thermocycler. Gibson assembly conditions were: 50° C. for 60 min and held at 12° C. until retrieval from the thermocycler. Ligated plasmids were transformed into TOP10 E. coli cells using the heat shock method. After incubation at 37° C. for 18-24 h, colonies were picked and expanded overnight in 5 mL LB broth supplemented with ampicillin (100 μg/mL) or kanamycin (100 μg/mL). DNA was extracted from colonies using a Zymo Research Plasmid Mini-prep Kit. DNA was subjected to restriction enzyme digestion to confirm gene insertion. Positive hits were further sequenced. See Table 1 and Table 2 for additional construct details.

TABLE 1

| Features | Plasmid Vector | Promoter | Expression in | Used In |
|---|---|---|---|---|
| NLS - HA - MS2 - SmBiT114 - | pCDNA 3.1/ pLenti | CMV | Mammalian | FIG. 1D-1G, |
| IRES - PP7 - LgBiT - FLAG | 3rd generation | | | FIG. 8B-8F |
| | | | | FIG. 10B-10G |
| | | | | FIG. 3A-3B, |
| | | | | FIG. 4A-4C |
| | | | | FIG. 5A-5B, |
| | | | | FIG. 6A-6B, |
| | | | | FIG. 7A-7E |
| | | | | FIG. 13 |
| NLS - HA - MS2 - SmBiT99 - | pCDNA 3.1 | CMV | Mammalian | FIG. 2A-2C |
| IRES - PP7 - LgBiT - FLAG | | | | |
| NLS - HA - MS2 - 3x | pCDNA 3.1 | CMV | Mammalian | FIG. 2A-2C |
| SmBiT99 - IRES - PP7 - | | | | |
| LgBiT - FLAG | | | | |
| SV40 NLS - HA - MS2 - 3x | pCDNA 3.1 | CMV | Mammalian | FIG. 2A-2C |
| SmBiT114 - IRES - PP7 - | | | | |
| LgBiT - FLAG | | | | |
| NLS - HA - MS2 - G4S - | pCDNA 3.1 | CMV | Mammalian | FIG. 7A-7E |
| SmBiT114 - IRES - PP7 - | | | | |
| G4S - LgBiT - FLAG | | | | |
| NLS - HA - MS2 - G4S - | pCDNA 3.1 | CMV | Mammalian | FIG. 7A-7E |
| SmBiT114 - IRES - PP7 - | | | | |
| G4Sx3 - LgBiT - FLAG | | | | |
| NLS - HA - MS2 - G4S - | pCDNA 3.1 | CMV | Mammalian | FIG. 7A-7E |
| SmBiT114 - IRES - PP7 - | | | | |
| G4Sx5 - LgBiT - FLAG | | | | |
| NLS - HA - MS2 - G4Sx3 - | pCDNA 3.1 | CMV | Mammalian | FIG. 7A-7E |
| SmBiT114 - IRES - PP7 - | | | | |
| G4S - LgBiT - FLAG | | | | |
| NLS - HA - MS2 - G4Sx3 - | pCDNA 3.1 | CMV | Mammalian | FIG. 7A-7E |
| SmBiT114 - IRES - PP7 - | | | | |
| G4Sx3 - LgBiT - FLAG | | | | |
| NLS - HA - MS2 - G4Sx3 - | pCDNA 3.1 | CMV | Mammalian | FIG. 7A-7E |
| SmBiT114 - IRES - PP7 - | | | | |
| G4Sx5 - LgBiT - FLAG | | | | |
| NLS - HA - MS2 - G4Sx5 - | pCDNA 3.1 | CMV | Mammalian | FIG. 7A-7E |
| SmBiT114 - IRES - PP7 - | | | | |
| G4S - LgBiT - FLAG | | | | |
| NLS - HA - MS2 - G4Sx5 - | pCDNA 3.1 | CMV | Mammalian | FIG. 7A-7E |
| SmBiT114 - IRES - PP7 - | | | | |
| G4Sx3 - LgBiT - FLAG | | | | |
| NLS - HA - MS2 - G4Sx5 - | pCDNA 3.1 | CMV | Mammalian | FIG. 7A-7E |
| SmBiT114 - IRES - PP7 - | | | | |
| G4Sx5 - LgBiT - FLAG | | | | |
| NLS - HA - MS2 - linker - | pCDNA 3.1 | CMV | Mammalian | FIG. 9A-9B, |
| SmBiT114 - IRES - PP7 - | | | | FIG. 4A-4H |
| linker - YeLgBiT - FLAG | | | | |
| NLS - HA - MS2 - linker - | pCDNA 3.1 | CMV | Mammalian | FIG. 9A-9B, |
| SmBiT114 - IRES - PP7 - | | | | FIG. 4A-4H |
| linker - LumiLgBiT - FLAG | | | | |
| NLS - HA - MS2 - FlucN - | pCDNA 3.1 | CMV | Mammalian | FIG. 9A-9B, |
| IRES - PP7 - FlucC - FLAG | | | | FIG. 4A-4H |
| BFP - MS2-3-PP7 | pCDNA 3.1 | CMV | Mammalian | FIG. 9A-9B, |
| | | | | FIG. 4A-4H |
| | | | | FIG. 7A-7E |
| BFP - MS2-3-PP7 (mut) | pCDNA 3.1 | CMV | Mammalian | FIG. 9A-9B, |
| | | | | FIG. 4A-4H |
| Staygold - MS2-3-PP7 | pCDNA 3.1 | CMV | Mammalian | FIG. 9A-9B, |
| | | | | FIG. 4A-4H |
| | | | | FIG. 13 |
| Staygold | pCDNA 3.1 | CMV | Mammalian | FIG. 9A-9B, |
| | | | | FIG. 4A-4H |
| | | | | FIG. 13 |

TABLE 2
Plasmid and RNA Sequences:

| | | SEQ |
|---|---|---|
| | | ID |
| | Sequence | NO: |
| CMV enhancer/ | cgttacataacttacggtaaatggcccgcctggctgaccgccca | 1 |
| promoter-NLS-HA | acgacccccgcccattgacgtcaataatgacgtatgttcccata | |
| -MCP-SmBIT114- | gtaacgccaatagggactttccattgacgtcaatgggtggagta | |
| IRES-PCP-LgBiT | tttacggtaaactgcccacttggcagtacatcaagtgtatcata | |
| -FLAG | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgcggattctagacgggatcccgccaccgtgaccg | |
| | gctaccgcctgtttgaggagattctgtaacgtacgacgcgtgga | |
| | tccctcccccccccctaacgttactggccgaagccgcttggaat | |
| | aaggccggtgtgcgtttgtctatatgttattttccaccatattg | |
| | ccgtcttttggcaatgtgagggcccggaaacctggccctgtctt | |
| | cttgacgagcattcctaggggtctttcccctctcgccaaaggaa | |
| | tgcaaggtctgttgaatgtcgtgaaggaagcagttcctctggaa | |
| | gcttcttgaagacaaacaacgtctgtagcgaccctttgcaggca | |
| | gcggaaccccccacctggcgacaggtgcctctgcggccaaaagc | |
| | cacgtgtataagatacacctgcaaaggcggcacaaccccagtgc | |
| | cacgttgtgagttggatagttgtggaaagagtcaaatggctctc | |
| | ctcaagcgtattcaacaaggggctgaaggatgcccagaaggtac | |
| | cccattgtatgggatctgatctggggcctcggtgcacatgcttt | |
| | acatgtgtttagtcgaggttaaaaaaacgtctaggccccccgaa | |
| | ccacggggacgtggttttcctttgaaaaacacgatgataatatg | |
| | gccacagcctgcaggatgggctccaaaaccatcgttctttcggt | |
| | cggcgaggctactcgcactctgactgagatccagtccaccgcag | |
| | accgtcagatcttcgaagagaaggtcgggcctctggtgggtcgg | |
| | ctgcgcctcacggcttcgctccgtcaaaacggagccaagaccgc | |
| | gtatcgcgtcaacctaaaactggatcaggcggacgtcgttgatt | |
| | ccggacttccgaaagtgcgctacactcaggtatggtcgcacgac | |
| | gtgacaatcgttgcgaatagcaccgaggcctcgcgcaaatcgtt | |
| | gtacgatttgaccaagtccctcgtcgcgacctcgcaggtcgaag | |
| | atcttgtcgtcaaccttgtgccgctgggccgtgcggatccaccg | |
| | gtatgcatcgccaccgccttcacactcgaagatttcgttgggga | |
| | ctgggaacagacagccgcctacaacctggaccaagtccttgaac | |
| | agggaggtgtgtccagtttgctgcagaatctcgccgtgtccgta | |
| | actccgatccaaaggattgtccggagcggtgaaaatgccctgaa | |
| | gatcgacatccatgtcatcatcccgtatgaaggtctgagcgccg | |
| | accaaatggcccagatcgaagaggtgtttaaggtggtgtaccct | |
| | gtggatgatttcacactcgaagatttcgttggggactgggaaca | |
| | gacagccgcctacaacctggaccaagtccttgaacagggaggtg | |
| | tgtccagtttgctgcagaatctcgccgtgtccgtaactccgatc | |
| | caaaggattgtccggagcggtgaaaatgccctgaagatcgacat | |
| | ccatgtcatcatcccgtatgaaggtctgagcgccgaccaaatgg | |
| | cccagatcgaagaggtgtttaaggtggtgtaccctgtggatgat | |
| CMV enhancer/ | cgttacataacttacggtaaatggcccgcctggctgaccgccca | 2 |
| promoter-NLS-HA | acgacccccgcccattgacgtcaataatgacgtatgttcccata | |
| -MCP-SmBIT99- | gtaacgccaatagggactttccattgacgtcaatgggtggagta | |
| IRES-PCP-LgBiT | tttacggtaaactgcccacttggcagtacatcaagtgtatcata | |
| -FLAG | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgcggattctagacgggatcccgccaccgtgaccg | |
| | gctaccgcctgtttgagaaaattagctaacgtacgacgcgtgga | |
| | tccctcccccccccctaacgttactggccgaagccgcttggaat | |
| | aaggccggtgtgcgtttgtctatatgttattttccaccatattg | |
| | ccgtcttttggcaatgtgagggcccggaaacctggccctgtctt | |
| | cttgacgagcattcctaggggtctttcccctctcgccaaaggaa | |
| | tgcaaggtctgttgaatgtcgtgaaggaagcagttcctctggaa | |
| | gcttcttgaagacaaacaacgtctgtagcgaccctttgcaggca | |
| | gcggaaccccccacctggcgacaggtgcctctgcggccaaaagc | |
| | cacgtgtataagatacacctgcaaaggcggcacaaccccagtgc | |
| | cacgttgtgagttggatagttgtggaaagagtcaaatggctctc | |
| | ctcaagcgtattcaacaaggggctgaaggatgcccagaaggtac | |
| | cccattgtatgggatctgatctggggcctcggtgcacatgcttt | |
| | acatgtgtttagtcgaggttaaaaaaacgtctaggccccccgaa | |
| | ccacggggacgtggttttcctttgaaaaacacgatgataatatg | |
| | gccacagcctgcaggatgggctccaaaaccatcgttctttcggt | |
| | cggcgaggctactcgcactctgactgagatccagtccaccgcag | |
| | accgtcagatcttcgaagagaaggtcgggcctctggtgggtcgg | |
| | ctgcgcctcacggcttcgctccgtcaaaacggagccaagaccgc | |
| | gtatcgcgtcaacctaaaactggatcaggcggacgtcgttgatt | |
| | ccggacttccgaaagtgcgctacactcaggtatggtcgcacgac | |
| | gtgacaatcgttgcgaatagcaccgaggcctcgcgcaaatcgtt | |
| | gtacgatttgaccaagtccctcgtcgcgacctcgcaggtcgaag | |
| | atcttgtcgtcaaccttgtgccgctgggccgtgcggatccaccg | |
| | gtatgcatcgccaccgccttcacactcgaagatttcgttgggga | |
| | ctgggaacagacagccgcctacaacctggaccaagtccttgaac | |
| | agggaggtgtgtccagtttgctgcagaatctcgccgtgtccgta | |
| | actccgatccaaaggattgtccggagcggtgaaaatgccctgaa | |
| | gatcgacatccatgtcatcatcccgtatgaaggtctgagcgccg | |
| | accaaatggcccagatcgaagaggtgtttaaggtggtgtaccct | |
| | gtggatgatcatcactttaaggtgatcctgccctatggcacact | |
| | ggtaatcgacggggttacgccgaacatgctgaactatttcggac | |
| | ggccgtatgaaggcatcgccgtgttcgacggcaaaaagatcact | |
| | gtaacagggaccctgtggaacggcaacaaaattatcgacgagcg | |
| | cctgatcacccccgacggctccatgctgttccgagtaaccatca | |
| | acagcctcgaggactacaaggacgacgatgacaa | |
| NLS-HA-MCP- | cgttacataacttacggtaaatggcccgcctggctgaccgccca | 3 |
| 3X SmBiT99-IRES | acgacccccgcccattgacgtcaataatgacgtatgttcccata | |
| -PCP-LgBiT- | gtaacgccaatagggactttccattgacgtcaatgggtggagta | |
| FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgcggattctagacgggatcccgccaccgtgaccg | |
| | gctaccgcctgtttgagaaaattagcgggagcggaagtggggtt | |
| | accggttatcgtctgttcgaaaaaatcagcgggtccggatcggg | |
| | tgtgacaggctatcgtctgtttgagaaaatttcctaacgtacga | |
| | cgcgtggatccctcccccccccctaacgttactggccgaagccg | |
| | cttggaataaggccggtgtgcgtttgtctatatgttattttcca | |
| | ccatattgccgtcttttggcaatgtgagggcccggaaacctggc | |
| | cctgtcttcttgacgagcattcctaggggtctttcccctctcgc | |
| | caaaggaatgcaaggtctgttgaatgtcgtgaaggaagcagttc | |
| | ctctggaagcttcttgaagacaaacaacgtctgtagcgaccctt | |
| | tgcaggcagcggaaccccccacctggcgacaggtgcctctgcgg | |
| | ccaaaagccacgtgtataagatacacctgcaaaggcggcacaac | |
| | cccagtgccacgttgtgagttggatagttgtggaaagagtcaaa | |
| | tggctctcctcaagcgtattcaacaaggggctgaaggatgccca | |
| | gaaggtaccccattgtatgggatctgatctggggcctcggtgca | |
| | catgctttacatgtgtttagtcgaggttaaaaaaacgtctaggc | |
| | cccccgaaccacggggacgtggttttcctttgaaaaacacgatg | |
| | ataatatggccacagcctgcaggatgggctccaaaaccatcgtt | |
| | ctttcggtcggcgaggctactcgcactctgactgagatccagtc | |
| | caccgcagaccgtcagatcttcgaagagaaggtcgggcctctgg | |
| | tgggtcggctgcgcctcacggcttcgctccgtcaaaacggagcc | |
| | aagaccgcgtatcgcgtcaacctaaaactggatcaggcggacgt | |
| | cgttgattccggacttccgaaagtgcgctacactcaggtatggt | |
| | cgcacgacgtgacaatcgttgcgaatagcaccgaggcctcgcgc | |
| | aaatcgttgtacgatttgaccaagtccctcgtcgcgacctcgca | |
| | ggtcgaagatcttgtcgtcaaccttgtgccgctgggccgtgcgg | |
| | atccaccggtatgcatcgccaccgccttcacactcgaagatttc | |
| | gttggggactgggaacagacagccgcctacaacctggaccaagt | |
| | ccttgaacagggaggtgtgtccagtttgctgcagaatctcgccg | |
| | tgtccgtaactccgatccaaaggattgtccggagcggtgaaaat | |
| | gccctgaagatcgacatccatgtcatcatcccgtatgaaggtct | |
| | gagcgccgaccaaatggcccagatcgaagaggtgtttaaggtgg | |
| | tgtaccctgtggatgatcatcactttaaggtgatcctgccctat | |
| | ggcacactggtaatcgacggggttacgccgaacatgctgaacta | |
| | tttcggacggccgtatgaaggcatcgccgtgttcgacggcaaaa | |
| | agatcactgtaacagggaccctgtggaacggcaacaaaattatc | |
| | gacgagcgcctgatcacccccgacggctccatgctgttccgagt | |
| | aaccatcaacagcctcgaggactacaaggacgacgatgacaag | |
| SV40 NLS-HA- | cgttacataacttacggtaaatggcccgcctggctgaccgccca | 4 |
| MCP-3x SmBIT114 | acgacccccgcccattgacgtcaataatgacgtatgttcccata | |
| -IRES-PCP- | gtaacgccaatagggactttccattgacgtcaatgggggagtat | |
| LgBIT-FLAG | ttacggtaaactgcccacttggcagtacatcaagtgtatcatat | |
| | gccaagtacgccccctattgacgtcaatgacggtaaatggcccg | |
| | cctggcattatgcccagtacatgaccttatgggactttcctact | |
| | tggcagtacatctacgtattagtcatcgctattaccatggtgat | |
| | gcggttttggcagtacatcaatgggcgtggatagcggtttgact | |
| | cacggggatttccaagtctccaccccattgacgtcaatgggagt | |
| | ttgttttggcaccaaaatcaacgggactttccaaaatgtcgtaa | |
| | caactccgccccattgacgcaaatgggcggtaggcgtgtacggt | |
| | gggaggtctatataagcagagctctctggctaactagagaaccc | |
| | actgcttactggcttatcgaaattaatacgactcactataggga | |
| | gacccaagcttatgggcccaaaaaagaaaagaaaagttggctac | |
| | ccctacgacgtgcccgactacgccatcgaaggccgccatatgct | |
| | agccgttaaaatggcttctaactttactcagttcgttctcgtcg | |
| | acaatggcggaactggcgacgtgactgtcgccccaagcaacttc | |
| | gctaacgggatcgctgaatggatcagctctaactcgcgttcaca | |
| | ggcttacaaagtaacctgtagcgttcgtcagagctctgcgcaga | |
| | atcgcaaatacaccatcaaagtcgaggtgcctaaaggcgcctgg | |
| | cgttcgtacttaaatatggaactaaccattccaattttcgccac | |
| | gaattccgactgcgagcttattgttaaggcaatgcaaggtctcc | |
| | taaaagatggaaacccgattccctcagcaatcgcagcaaactcc | |
| | ggcatctacgcggattctagacgggatcccgccaccgtgaccgg | |
| | ctaccgcctgtttgaggagattctggggagcggaagtggggtga | |
| | ccggatatcggctgtttgaggagattctggggtccggatcgggt | |
| | gtgacagggtatcgcctgttcgaggaaatcctgtaacgtacgac | |
| | gcgtggatccctcccccccccctaacgttactggccgaagccgc | |
| | ttggaataaggccggtgtgcgtttgtctatatgttattttccac | |
| | catattgccgtcttttggcaatgtgagggcccggaaacctggcc | |
| | ctgtcttcttgacgagcattcctaggggtctttcccctctcgcc | |
| | aaaggaatgcaaggtctgttgaatgtcgtgaaggaagcagttcc | |
| | tctggaagcttcttgaagacaaacaacgtctgtagcgacccttt | |
| | gcaggcagcggaaccccccacctggcgacaggtgcctctgcggc | |
| | caaaagccacgtgtataagatacacctgcaaaggcggcacaacc | |
| | ccagtgccacgttgtgagttggatagttgtggaaagagtcaaat | |
| | ggctctcctcaagcgtattcaacaaggggctgaaggatgcccag | |
| | aaggtaccccattgtatgggatctgatctggggcctcggtgcac | |
| | atgctttacatgtgtttagtcgaggttaaaaaaacgtctaggcc | |
| | ccccgaaccacggggacgtggttttcctttgaaaaacacgatga | |
| | taatatggccacagcctgcaggatgggctccaaaaccatcgttc | |
| | tttcggtcggcgaggctactcgcactctgactgagatccagtcc | |
| | accgcagaccgtcagatcttcgaagagaaggtcgggcctctggt | |
| | gggtcggctgcgcctcacggcttcgctccgtcaaaacggagcca | |
| | agaccgcgtatcgcgtcaacctaaaactggatcaggcggacgtc | |
| | gttgattccggacttccgaaagtgcgctacactcaggtatggtc | |
| | gcacgacgtgacaatcgttgcgaatagcaccgaggcctcgcgca | |
| | aatcgttgtacgatttgaccaagtccctcgtcgcgacctcgcag | |
| | gtcgaagatcttgtcgtcaaccttgtgccgctgggccgtgcgga | |
| | tccaccggtatgcatcgccaccgccttcacactcgaagatttcg | |
| | ttggggactgggaacagacagccgcctacaacctggaccaagtc | |
| | cttgaacagggaggtgtgtccagtttgctgcagaatctcgccgt | |
| | gtccgtaactccgatccaaaggattgtccggagcggtgaaaatg | |
| | ccctgaagatcgacatccatgtcatcatcccgtatgaaggtctg | |
| | agcgccgaccaaatggcccagatcgaagaggtgtttaaggtggt | |
| | gtaccctgtggatgatcatcactttaaggtgatcctgccctatg | |
| | gcacactggtaatcgacggggttacgccgaacatgctgaactat | |
| | ttcggacggccgtatgaaggcatcgccgtgttcgacggcaaaaa | |
| | gatcactgtaacagggaccctgtggaacggcaacaaaattatcg | |
| | acgagcgcctgatcacccccgacggctccatgctgttccgagta | |
| | accatcaacagcctcgaggactacaaggacgacgatgacaag | |
| NLS-HA-MCP- | cgttacataacttacggtaaatggcccgcctggctgaccgccca | 5 |
| G4S-SmBIT114- | acgacccccgcccattgacgtcaataatgacgtatgttcccata | |
| IRES-PCP-G4S- | gtaacgccaatagggactttccattgacgtcaatgggtggagta | |
| LgBIT-FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgatatcggcggcggaggctctgtgaccggctacc | |
| | gcctgtttgaggagattctgtaacgtacgacgcgtggatccctc | |
| | ccccccccctaacgttactggccgaagccgcttggaataaggcc | |
| | ggtgtgcgtttgtctatatgttattttccaccatattgccgtct | |
| | tttggcaatgtgagggcccggaaacctggccctgtcttcttgac | |
| | gagcattcctaggggtctttcccctctcgccaaaggaatgcaag | |
| | gtctgttgaatgtcgtgaaggaagcagttcctctggaagcttct | |
| | tgaagacaaacaacgtctgtagcgaccctttgcaggcagcggaa | |
| | ccccccacctggcgacaggtgcctctgcggccaaaagccacgtg | |
| | tataagatacacctgcaaaggcggcacaaccccagtgccacgtt | |
| | gtgagttggatagttgtggaaagagtcaaatggctctcctcaag | |
| | cgtattcaacaaggggctgaaggatgcccagaaggtaccccatt | |
| | gtatgggatctgatctggggcctcggtgcacatgctttacatgt | |
| | gtttagtcgaggttaaaaaaacgtctaggccccccgaaccacgg | |
| | ggacgtggttttcctttgaaaaacacgatgataatatggccaca | |
| | gcctgcaggatgggctccaaaaccatcgttctttcggtcggcga | |
| | ggctactcgcactctgactgagatccagtccaccgcagaccgtc | |
| | agatcttcgaagagaaggtcgggcctctggtgggtcggctgcgc | |
| | ctcacggcttcgctccgtcaaaacggagccaagaccgcgtatcg | |
| | cgtcaacctaaaactggatcaggcggacgtcgttgattccggac | |
| | ttccgaaagtgcgctacactcaggtatggtcgcacgacgtgaca | |
| | atcgttgcgaatagcaccgaggcctcgcgcaaatcgttgtacga | |
| | tttgaccaagtccctcgtcgcgacctcgcaggtcgaagatcttg | |
| | tcgtcaaccttgtgccgctgggccgtatcgatggcggaggcggg | |
| | tctttcacactcgaagatttcgttggggactgggaacagacagc | |
| | cgcctacaacctggaccaagtccttgaacagggaggtgtgtcca | |
| | gtttgctgcagaatctcgccgtgtccgtaactccgatccaaagg | |
| | attgtccggagcggtgaaaatgccctgaagatcgacatccatgt | |
| | catcatcccgtatgaaggtctgagcgccgaccaaatggcccaga | |
| | tcgaagaggtgtttaaggtggtgtaccctgtggatgatcatcac | |
| | tttaaggtgatcctgccctatggcacactggtaatcgacggggt | |
| | tacgccgaacatgctgaactatttcggacggccgtatgaaggca | |
| | tcgccgtgttcgacggcaaaaagatcactgtaacagggaccctg | |
| | tggaacggcaacaaaattatcgacgagcgcctgatcacccccga | |
| | cggctccatgctgttccgagtaaccatcaacagcctcgaggact | |
| | acaaggacgacgatgacaag | |
| NLS-HA-MCP- | cgttacataacttacggtaaatggcccgcctggctgaccgccca | 6 |
| G4S-SmBiT114- | acgacccccgcccattgacgtcaataatgacgtatgttcccata | |
| IRES-PCP-G4Sx3 | gtaacgccaatagggactttccattgacgtcaatgggtggagta | |
| -LgBiT-FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgatatcggcggcggaggctctgtgaccggctacc | |
| | gcctgtttgaggagattctgtaacgtacgacgcgtggatccctc | |
| | ccccccccctaacgttactggccgaagccgcttggaataaggcc | |
| | ggtgtgcgtttgtctatatgttattttccaccatattgccgtct | |
| | tttggcaatgtgagggcccggaaacctggccctgtcttcttgac | |
| | gagcattcctaggggtctttcccctctcgccaaaggaatgcaag | |
| | gtctgttgaatgtcgtgaaggaagcagttcctctggaagcttct | |
| | tgaagacaaacaacgtctgtagcgaccctttgcaggcagcggaa | |
| | ccccccacctggcgacaggtgcctctgcggccaaaagccacgtg | |
| | tataagatacacctgcaaaggcggcacaaccccagtgccacgtt | |
| | gtgagttggatagttgtggaaagagtcaaatggctctcctcaag | |
| | cgtattcaacaaggggctgaaggatgcccagaaggtaccccatt | |
| | gtatgggatctgatctggggcctcggtgcacatgctttacatgt | |
| | gtttagtcgaggttaaaaaaacgtctaggccccccgaaccacgg | |
| | ggacgtggttttcctttgaaaaacacgatgataatatggccaca | |
| | gcctgcaggatgggctccaaaaccatcgttctttcggtcggcga | |
| | ggctactcgcactctgactgagatccagtccaccgcagaccgtc | |
| | agatcttcgaagagaaggtcgggcctctggtgggtcggctgcgc | |
| | ctcacggcttcgctccgtcaaaacggagccaagaccgcgtatcg | |
| | cgtcaacctaaaactggatcaggcggacgtcgttgattccggac | |
| | ttccgaaagtgcgctacactcaggtatggtcgcacgacgtgaca | |
| | atcgttgcgaatagcaccgaggcctcgcgcaaatcgttgtacga | |
| | tttgaccaagtccctcgtcgcgacctcgcaggtcgaagatcttg | |
| | tcgtcaaccttgtgccgctgggccgtatcgatggcggaggcggg | |
| | tctggcggcggcggcagcggtggaggaggttctttcacactcga | |
| | agatttcgttggggactgggaacagacagccgcctacaacctgg | |
| | accaagtccttgaacagggaggtgtgtccagtttgctgcagaat | |
| | ctcgccgtgtccgtaactccgatccaaaggattgtccggagcgg | |
| | tgaaaatgccctgaagatcgacatccatgtcatcatcccgtatg | |
| | aaggtctgagcgccgaccaaatggcccagatcgaagaggtgttt | |
| | aaggtggtgtaccctgtggatgatcatcactttaaggtgatcct | |
| | gccctatggcacactggtaatcgacggggttacgccgaacatgc | |
| | tgaactatttcggacggccgtatgaaggcatcgccgtgttcgac | |
| | ggcaaaaagatcactgtaacagggaccctgtggaacggcaacaa | |
| | aattatcgacgagcgcctgatcacccccgacggctccatgctgt | |
| | tccgagtaaccatcaacagcctcgaggactacaaggacgacgat | |
| | gacaag | |
| NLS-HA-MCP- | cgttacataacttacggtaaatggcccgcctggctgaccgccca | 7 |
| G4S-SmBIT114- | acgacccccgcccattgacgtcaataatgacgtatgttcccata | |
| IRES-PCP-G4Sx5 | gtaacgccaatagggactttccattgacgtcaatgggtggagta | |
| -LgBiT-FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgatatcggcggcggaggctctgtgaccggctacc | |
| | gcctgtttgaggagattctgtaacgtacgacgcgtggatccctc | |
| | ccccccccctaacgttactggccgaagccgcttggaataaggcc | |
| | ggtgtgcgtttgtctatatgttattttccaccatattgccgtct | |
| | tttggcaatgtgagggcccggaaacctggccctgtcttcttgac | |
| | gagcattcctaggggtctttcccctctcgccaaaggaatgcaag | |
| | gtctgttgaatgtcgtgaaggaagcagttcctctggaagcttct | |
| | tgaagacaaacaacgtctgtagcgaccctttgcaggcagcggaa | |
| | ccccccacctggcgacaggtgcctctgcggccaaaagccacgtg | |
| | tataagatacacctgcaaaggcggcacaaccccagtgccacgtt | |
| | gtgagttggatagttgtggaaagagtcaaatggctctcctcaag | |
| | cgtattcaacaaggggctgaaggatgcccagaaggtaccccatt | |
| | gtatgggatctgatctggggcctcggtgcacatgctttacatgt | |
| | gtttagtcgaggttaaaaaaacgtctaggccccccgaaccacgg | |
| | ggacgtggttttcctttgaaaaacacgatgataatatggccaca | |
| | gcctgcaggatgggctccaaaaccatcgttctttcggtcggcga | |
| | ggctactcgcactctgactgagatccagtccaccgcagaccgtc | |
| | agatcttcgaagagaaggtcgggcctctggtgggtcggctgcgc | |
| | ctcacggcttcgctccgtcaaaacggagccaagaccgcgtatcg | |
| | cgtcaacctaaaactggatcaggcggacgtcgttgattccggac | |
| | ttccgaaagtgcgctacactcaggtatggtcgcacgacgtgaca | |
| | atcgttgcgaatagcaccgaggcctcgcgcaaatcgttgtacga | |
| | tttgaccaagtccctcgtcgcgacctcgcaggtcgaagatcttg | |
| | tcgtcaaccttgtgccgctgggccgtatcgatggcggaggcggg | |
| | tctggtggagggggatctggaggtggtgggtctggcggcggcgg | |
| | cagcggtggaggaggttctttcacactcgaagatttcgttgggg | |
| | actgggaacagacagccgcctacaacctggaccaagtccttgaa | |
| | cagggaggtgtgtccagtttgctgcagaatctcgccgtgtccgt | |
| | aactccgatccaaaggattgtccggagcggtgaaaatgccctga | |
| | agatcgacatccatgtcatcatcccgtatgaaggtctgagcgcc | |
| | gaccaaatggcccagatcgaagaggtgtttaaggtggtgtaccc | |
| | tgtggatgatcatcactttaaggtgatcctgccctatggcacac | |
| | tggtaatcgacggggttacgccgaacatgctgaactatttcgga | |
| | cggccgtatgaaggcatcgccgtgttcgacggcaaaaagatcac | |
| | tgtaacagggaccctgtggaacggcaacaaaattatcgacgagc | |
| | gcctgatcacccccgacggctccatgctgttccgagtaaccatc | |
| | aacagcctcgaggactacaaggacgacgatgacaag | |
| NLS-HA-MCP- | cgttacataacttacggtaaatggcccgcctggctgaccgccca | 8 |
| G4SX3-SmBIT114 | acgacccccgcccattgacgtcaataatgacgtatgttcccata | |
| -IRES-PCP-G4S | gtaacgccaatagggactttccattgacgtcaatgggtggagta | |
| -LgBiT-FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgatatcggcggcggaggctctggaggcggcggaa | |
| | gcggtggggggggagcgtgaccggctaccgcctgtttgaggaga | |
| | ttctgtaacgtacgacgcgtggatccctcccccccccctaacgt | |
| | tactggccgaagccgcttggaataaggccggtgtgcgtttgtct | |
| | atatgttattttccaccatattgccgtcttttggcaatgtgagg | |
| | gcccggaaacctggccctgtcttcttgacgagcattcctagggg | |
| | tctttcccctctcgccaaaggaatgcaaggtctgttgaatgtcg | |
| | tgaaggaagcagttcctctggaagcttcttgaagacaaacaacg | |
| | tctgtagcgaccctttgcaggcagcggaaccccccacctggcga | |
| | caggtgcctctgcggccaaaagccacgtgtataagatacacctg | |
| | caaaggcggcacaaccccagtgccacgttgtgagttggatagtt | |
| | gtggaaagagtcaaatggctctcctcaagcgtattcaacaaggg | |
| | gctgaaggatgcccagaaggtaccccattgtatgggatctgatc | |
| | tggggcctcggtgcacatgctttacatgtgtttagtcgaggtta | |
| | aaaaaacgtctaggccccccgaaccacggggacgtggttttcct | |
| | ttgaaaaacacgatgataatatggccacagcctgcaggatgggc | |
| | tccaaaaccatcgttctttcggtcggcgaggctactcgcactct | |
| | gactgagatccagtccaccgcagaccgtcagatcttcgaagaga | |
| | aggtcgggcctctggtgggtcggctgcgcctcacggcttcgctc | |
| | cgtcaaaacggagccaagaccgcgtatcgcgtcaacctaaaact | |
| | ggatcaggcggacgtcgttgattccggacttccgaaagtgcgct | |
| | acactcaggtatggtcgcacgacgtgacaatcgttgcgaatagc | |
| | accgaggcctcgcgcaaatcgttgtacgatttgaccaagtccct | |
| | cgtcgcgacctcgcaggtcgaagatcttgtcgtcaaccttgtgc | |
| | cgctgggccgtatcgatggcggaggcgggtctttcacactcgaa | |
| | gatttcgttggggactgggaacagacagccgcctacaacctgga | |
| | ccaagtccttgaacagggaggtgtgtccagtttgctgcagaatc | |
| | tcgccgtgtccgtaactccgatccaaaggattgtccggagcggt | |
| | gaaaatgccctgaagatcgacatccatgtcatcatcccgtatga | |
| | aggtctgagcgccgaccaaatggcccagatcgaagaggtgttta | |
| | aggtggtgtaccctgtggatgatcatcactttaaggtgatcctg | |
| | ccctatggcacactggtaatcgacggggttacgccgaacatgct | |
| | gaactatttcggacggccgtatgaaggcatcgccgtgttcgacg | |
| | gcaaaaagatcactgtaacagggaccctgtggaacggcaacaaa | |
| | attatcgacgagcgcctgatcacccccgacggctccatgctgtt | |
| | ccgagtaaccatcaacagcctcgaggactacaaggacgacgatg | |
| | acaag | |
| NLS-HA-MCP- | cgttacataacttacggtaaatggcccgcctggctgaccgccca | 9 |
| G4Sx3-SmBIT114- | acgacccccgcccattgacgtcaataatgacgtatgttcccata | |
| IRES-PCP- | gtaacgccaatagggactttccattgacgtcaatgggtggagta | |
| G4Sx3-LgBiT- | tttacggtaaactgcccacttggcagtacatcaagtgtatcata | |
| FLAG | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggggtaggcgtgtacggt | |
| | gggaggtctatataagcagagctctctggctaactagagaaccc | |
| | actgcttactggcttatcgaaattaatacgactcactataggga | |
| | gacccaagcttatgggcccaaaaaagaaaagaaaagttggctac | |
| | ccctacgacgtgcccgactacgccatcgaaggccgccatatgct | |
| | agccgttaaaatggcttctaactttactcagttcgttctcgtcg | |
| | acaatggcggaactggcgacgtgactgtcgccccaagcaacttc | |
| | gctaacgggatcgctgaatggatcagctctaactcgcgttcaca | |
| | ggcttacaaagtaacctgtagcgttcgtcagagctctgcgcaga | |
| | atcgcaaatacaccatcaaagtcgaggtgcctaaaggcgcctgg | |
| | cgttcgtacttaaatatggaactaaccattccaattttcgccac | |
| | gaattccgactgcgagcttattgttaaggcaatgcaaggtctcc | |
| | taaaagatggaaacccgattccctcagcaatcgcagcaaactcc | |
| | ggcatctacgatatcggcggcggaggctctggaggcggcggaag | |
| | cggtgggggtgggagcgtgaccggctaccgcctgtttgaggaga | |
| | ttctgtaacgtacgacgcgtggatccctcccccccccctaacgt | |
| | tactggccgaagccgcttggaataaggccggtgtgcgtttgtct | |
| | atatgttattttccaccatattgccgtcttttggcaatgtgagg | |
| | gcccggaaacctggccctgtcttcttgacgagcattcctagggg | |
| | tctttcccctctcgccaaaggaatgcaaggtctgttgaatgtcg | |
| | tgaaggaagcagttcctctggaagcttcttgaagacaaacaacg | |
| | tctgtagcgaccctttgcaggcagcggaaccccccacctggcga | |
| | caggtgcctctgcggccaaaagccacgtgtataagatacacctg | |
| | caaaggcggcacaaccccagtgccacgttgtgagttggatagtt | |
| | gtggaaagagtcaaatggctctcctcaagcgtattcaacaaggg | |
| | gctgaaggatgcccagaaggtaccccattgtatgggatctgatc | |
| | tggggcctcggtgcacatgctttacatgtgtttagtcgaggtta | |
| | aaaaaacgtctaggccccccgaaccacggggacgtggttttcct | |
| | ttgaaaaacacgatgataatatggccacagcctgcaggatgggc | |
| | tccaaaaccatcgttctttcggtcggcgaggctactcgcactct | |
| | gactgagatccagtccaccgcagaccgtcagatcttcgaagaga | |
| | aggtcgggcctctggtgggtcggctgcgcctcacggcttcgctc | |
| | cgtcaaaacggagccaagaccgcgtatcgcgtcaacctaaaact | |
| | ggatcaggcggacgtcgttgattccggacttccgaaagtgcgct | |
| | acactcaggtatggtcgcacgacgtgacaatcgttgcgaatagc | |
| | accgaggcctcgcgcaaatcgttgtacgatttgaccaagtccct | |
| | cgtcgcgacctcgcaggtcgaagatcttgtcgtcaaccttgtgc | |
| | cgctgggccgtatcgatggcggaggcgggtctggcggcggcggc | |
| | agcggtggaggaggttctttcacactcgaagatttcgttgggga | |
| | ctgggaacagacagccgcctacaacctggaccaagtccttgaac | |
| | agggaggtgtgtccagtttgctgcagaatctcgccgtgtccgta | |
| | actccgatccaaaggattgtccggagcggtgaaaatgccctgaa | |
| | gatcgacatccatgtcatcatcccgtatgaaggtctgagcgccg | |
| | accaaatggcccagatcgaagaggtgtttaaggtggtgtaccct | |
| | gtggatgatcatcactttaaggtgatcctgccctatggcacact | |
| | ggtaatcgacggggttacgccgaacatgctgaactatttcggac | |
| | ggccgtatgaaggcatcgccgtgttcgacggcaaaaagatcact | |
| | gtaacagggaccctgtggaacggcaacaaaattatcgacgagcg | |
| | cctgatcacccccgacggctccatgctgttccgagtaaccatca | |
| | acagcctcgaggactacaaggacgacgatgacaag | |
| NLS-HA-MCP- | cgttacataacttacggtaaatggcccgcctggctgaccgccca | 10 |
| G4SX3-SmBIT114 | acgacccccgcccattgacgtcaataatgacgtatgttcccata | |
| -IRES-PCP- | gtaacgccaatagggactttccattgacgtcaatgggtggagta | |
| G4SX5-LgBiT- | tttacggtaaactgcccacttggcagtacatcaagtgtatcata | |
| FLAG | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgatatcggcggcggaggctctggaggcggcggaa | |
| | gcggtggggggggagcgtgaccggctaccgcctgtttgaggaga | |
| | ttctgtaacgtacgacgcgtggatccctcccccccccctaacgt | |
| | tactggccgaagccgcttggaataaggccggtgtgcgtttgtct | |
| | atatgttattttccaccatattgccgtcttttggcaatgtgagg | |
| | gcccggaaacctggccctgtcttcttgacgagcattcctagggg | |
| | tctttcccctctcgccaaaggaatgcaaggtctgttgaatgtcg | |
| | tgaaggaagcagttcctctggaagcttcttgaagacaaacaacg | |
| | tctgtagcgaccctttgcaggcagcggaaccccccacctggcga | |
| | caggtgcctctgcggccaaaagccacgtgtataagatacacctg | |
| | caaaggcggcacaaccccagtgccacgttgtgagttggatagtt | |
| | gtggaaagagtcaaatggctctcctcaagcgtattcaacaaggg | |
| | gctgaaggatgcccagaaggtaccccattgtatgggatctgatc | |
| | tggggcctcggtgcacatgctttacatgtgtttagtcgaggtta | |
| | aaaaaacgtctaggccccccgaaccacggggacgtggttttcct | |
| | ttgaaaaacacgatgataatatggccacagcctgcaggatgggc | |
| | tccaaaaccatcgttctttcggtcggcgaggctactcgcactct | |
| | gactgagatccagtccaccgcagaccgtcagatcttcgaagaga | |
| | aggtcgggcctctggtgggtcggctgcgcctcacggcttcgctc | |
| | cgtcaaaacggagccaagaccgcgtatcgcgtcaacctaaaact | |
| | ggatcaggcggacgtcgttgattccggacttccgaaagtgcgct | |
| | acactcaggtatggtcgcacgacgtgacaatcgttgcgaatagc | |
| | accgaggcctcgcgcaaatcgttgtacgatttgaccaagtccct | |
| | cgtcgcgacctcgcaggtcgaagatcttgtcgtcaaccttgtgc | |
| | cgctgggccgtatcgatggcggaggcgggtctggtggaggggga | |
| | tctggaggtggtgggtctggcggcggcggcagcggtggaggagg | |
| | ttctttcacactcgaagatttcgttggggactgggaacagacag | |
| | ccgcctacaacctggaccaagtccttgaacagggaggtgtgtcc | |
| | agtttgctgcagaatctcgccgtgtccgtaactccgatccaaag | |
| | gattgtccggagcggtgaaaatgccctgaagatcgacatccatg | |
| | tcatcatcccgtatgaaggtctgagcgccgaccaaatggcccag | |
| | atcgaagaggtgtttaaggtggtgtaccctgtggatgatcatca | |
| | ctttaaggtgatcctgccctatggcacactggtaatcgacgggg | |
| | ttacgccgaacatgctgaactatttcggacggccgtatgaaggc | |
| | atcgccgtgttcgacggcaaaaagatcactgtaacagggaccct | |
| | gtggaacggcaacaaaattatcgacgagcgcctgatcacccccg | |
| | acggctccatgctgttccgagtaaccatcaacagcctcgaggac | |
| | tacaaggacgacgatgacaag | |
| NLS-HA-MCP- | cgttacataacttacggtaaatggcccgcctggctgaccgccca | 11 |
| G4SX5-SmBiT114 | acgacccccgcccattgacgtcaataatgacgtatgttcccata | |
| -IRES-PCP-G4S | gtaacgccaatagggactttccattgacgtcaatgggtggagta | |
| -LgBiT-FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgatatcggcggcggaggctctggtggaggaggtt | |
| | ctggcggcggtggttccggaggcggcggaagcggtgggggggga | |
| | gcgtgaccggctaccgcctgtttgaggagattctgtaacgtacg | |
| | acgcgtggatccctcccccccccctaacgttactggccgaagcc | |
| | gcttggaataaggccggtgtgcgtttgtctatatgttattttcc | |
| | accatattgccgtcttttggcaatgtgagggcccggaaacctgg | |
| | ccctgtcttcttgacgagcattcctaggggtctttcccctctcg | |
| | ccaaaggaatgcaaggtctgttgaatgtcgtgaaggaagcagtt | |
| | cctctggaagcttcttgaagacaaacaacgtctgtagcgaccct | |
| | ttgcaggcagcggaaccccccacctggcgacaggtgcctctgcg | |
| | gccaaaagccacgtgtataagatacacctgcaaaggcggcacaa | |
| | ccccagtgccacgttgtgagttggatagttgtggaaagagtcaa | |
| | atggctctcctcaagcgtattcaacaaggggctgaaggatgccc | |
| | agaaggtaccccattgtatgggatctgatctggggcctcggtgc | |
| | acatgctttacatgtgtttagtcgaggttaaaaaaacgtctagg | |
| | ccccccgaaccacggggacgtggttttcctttgaaaaacacgat | |
| | gataatatggccacagcctgcaggatgggctccaaaaccatcgt | |
| | tctttcggtcggcgaggctactcgcactctgactgagatccagt | |
| | ccaccgcagaccgtcagatcttcgaagagaaggtcgggcctctg | |
| | gtgggtcggctgcgcctcacggcttcgctccgtcaaaacggagc | |
| | caagaccgcgtatcgcgtcaacctaaaactggatcaggcggacg | |
| | tcgttgattccggacttccgaaagtgcgctacactcaggtatgg | |
| | tcgcacgacgtgacaatcgttgcgaatagcaccgaggcctcgcg | |
| | caaatcgttgtacgatttgaccaagtccctcgtcgcgacctcgc | |
| | aggtcgaagatcttgtcgtcaaccttgtgccgctgggccgtatc | |
| | gatggcggaggcgggtctttcacactcgaagatttcgttgggga | |
| | ctgggaacagacagccgcctacaacctggaccaagtccttgaac | |
| | agggaggtgtgtccagtttgctgcagaatctcgccgtgtccgta | |
| | actccgatccaaaggattgtccggagcggtgaaaatgccctgaa | |
| | gatcgacatccatgtcatcatcccgtatgaaggtctgagcgccg | |
| | accaaatggcccagatcgaagaggtgtttaaggtggtgtaccct | |
| | gtggatgatcatcactttaaggtgatcctgccctatggcacact | |
| | ggtaatcgacggggttacgccgaacatgctgaactatttcggac | |
| | ggccgtatgaaggcatcgccgtgttcgacggcaaaaagatcact | |
| | gtaacagggaccctgtggaacggcaacaaaattatcgacgagcg | |
| | cctgatcacccccgacggctccatgctgttccgagtaaccatca | |
| | acagcctcgaggactacaaggacgacgatgacaag | |
| NLS-HA-MCP- | cgttacataacttacggtaaatggcccgcctggctgaccgccca | 12 |
| G4Sx5-SmBIT114- | acgacccccgcccattgacgtcaataatgacgtatgttcccata | |
| IRES-PCP-G4Sx3 | gtaacgccaatagggactttccattgacgtcaatgggtggagta | |
| -LgBiT-FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgatatcggcggcggaggctctggtggaggaggtt | |
| | ctggcggcggtggttccggaggcggcggaagcggtgggggggga | |
| | gcgtgaccggctaccgcctgtttgaggagattctgtaacgtacg | |
| | acgcgtggatccctcccccccccctaacgttactggccgaagcc | |
| | gcttggaataaggccggtgtgcgtttgtctatatgttattttcc | |
| | accatattgccgtcttttggcaatgtgagggcccggaaacctgg | |
| | ccctgtcttcttgacgagcattcctaggggtctttcccctctcg | |
| | ccaaaggaatgcaaggtctgttgaatgtcgtgaaggaagcagtt | |
| | cctctggaagcttcttgaagacaaacaacgtctgtagcgaccct | |
| | ttgcaggcagcggaaccccccacctggcgacaggtgcctctgcg | |
| | gccaaaagccacgtgtataagatacacctgcaaaggcggcacaa | |
| | ccccagtgccacgttgtgagttggatagttgtggaaagagtcaa | |
| | atggctctcctcaagcgtattcaacaaggggctgaaggatgccc | |
| | agaaggtaccccattgtatgggatctgatctggggcctcggtgc | |
| | acatgctttacatgtgtttagtcgaggttaaaaaaacgtctagg | |
| | ccccccgaaccacggggacgtggttttcctttgaaaaacacgat | |
| | gataatatggccacagcctgcaggatgggctccaaaaccatcgt | |
| | tctttcggtcggcgaggctactcgcactctgactgagatccagt | |
| | ccaccgcagaccgtcagatcttcgaagagaaggtcgggcctctg | |
| | gtgggtcggctgcgcctcacggcttcgctccgtcaaaacggagc | |
| | caagaccgcgtatcgcgtcaacctaaaactggatcaggcggacg | |
| | tcgttgattccggacttccgaaagtgcgctacactcaggtatgg | |
| | tcgcacgacgtgacaatcgttgcgaatagcaccgaggcctcgcg | |
| | caaatcgttgtacgatttgaccaagtccctcgtcgcgacctcgc | |
| | aggtcgaagatcttgtcgtcaaccttgtgccgctgggccgtatc | |
| | gatggcggagggggtctggcggcggcggcagcggtggaggaggt | |
| | tctttcacactcgaagatttcgttggggactgggaacagacagc | |
| | cgcctacaacctggaccaagtccttgaacagggaggtgtgtcca | |
| | gtttgctgcagaatctcgccgtgtccgtaactccgatccaaagg | |
| | attgtccggagcggtgaaaatgccctgaagatcgacatccatgt | |
| | catcatcccgtatgaaggtctgagcgccgaccaaatggcccaga | |
| | tcgaagaggtgtttaaggtggtgtaccctgtggatgatcatcac | |
| | tttaaggtgatcctgccctatggcacactggtaatcgacggggt | |
| | tacgccgaacatgctgaactatttcggacggccgtatgaaggca | |
| | tcgccgtgttcgacggcaaaaagatcactgtaacagggaccctg | |
| | tggaacggcaacaaaattatcgacgagcgcctgatcacccccga | |
| | cggctccatgctgttccgagtaaccatcaacagcctcgaggact | |
| | acaaggacgacgatgacaag | |
| NLS-HA-MCP- | cgttacataacttacggtaaatggcccgcctggctgaccgccca | 13 |
| G4Sx5-SmBIT114- | acgacccccgcccattgacgtcaataatgacgtatgttcccata | |
| IRES-PCP-G4Sx5 | gtaacgccaatagggactttccattgacgtcaatgggtggagta | |
| -LgBiT-FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgatatcggcggcggaggctctggtggaggaggtt | |
| | ctggcggcggtggttccggaggcggcggaagcggtgggggggga | |
| | gcgtgaccggctaccgcctgtttgaggagattctgtaacgtacg | |
| | acgcgtggatccctcccccccccctaacgttactggccgaagcc | |
| | gcttggaataaggccggtgtgcgtttgtctatatgttattttcc | |
| | accatattgccgtcttttggcaatgtgagggcccggaaacctgg | |
| | ccctgtcttcttgacgagcattcctaggggtctttcccctctcg | |
| | ccaaaggaatgcaaggtctgttgaatgtcgtgaaggaagcagtt | |
| | cctctggaagcttcttgaagacaaacaacgtctgtagcgaccct | |
| | ttgcaggcagcggaaccccccacctggcgacaggtgcctctgcg | |
| | gccaaaagccacgtgtataagatacacctgcaaaggcggcacaa | |
| | ccccagtgccacgttgtgagttggatagttgtggaaagagtcaa | |
| | atggctctcctcaagcgtattcaacaaggggctgaaggatgccc | |
| | agaaggtaccccattgtatgggatctgatctggggcctcggtgc | |
| | acatgctttacatgtgtttagtcgaggttaaaaaaacgtctagg | |
| | ccccccgaaccacggggacgtggttttcctttgaaaaacacgat | |
| | gataatatggccacagcctgcaggatgggctccaaaaccatcgt | |
| | tctttcggtcggcgaggctactcgcactctgactgagatccagt | |
| | ccaccgcagaccgtcagatcttcgaagagaaggtcgggcctctg | |
| | gtgggtcggctgcgcctcacggcttcgctccgtcaaaacggagc | |
| | caagaccgcgtatcgcgtcaacctaaaactggatcaggcggacg | |
| | tcgttgattccggacttccgaaagtgcgctacactcaggtatgg | |
| | tcgcacgacgtgacaatcgttgcgaatagcaccgaggcctcgcg | |
| | caaatcgttgtacgatttgaccaagtccctcgtcgcgacctcgc | |
| | aggtcgaagatcttgtcgtcaaccttgtgccgctgggccgtatc | |
| | gatggcggaggcgggtctggtggagggggatctggaggtggtgg | |
| | gtctggcggcggcggcagcggtggaggaggttctttcacactcg | |
| | aagatttcgttggggactgggaacagacagccgcctacaacctg | |
| | gaccaagtccttgaacagggaggtgtgtccagtttgctgcagaa | |
| | tctcgccgtgtccgtaactccgatccaaaggattgtccggagcg | |
| | gtgaaaatgccctgaagatcgacatccatgtcatcatcccgtat | |
| | gaaggtctgagcgccgaccaaatggcccagatcgaagaggtgtt | |
| | taaggtggtgtaccctgtggatgatcatcactttaaggtgatcc | |
| | tgccctatggcacactggtaatcgacggggttacgccgaacatg | |
| | ctgaactatttcggacggccgtatgaaggcatcgccgtgttcga | |
| | cggcaaaaagatcactgtaacagggaccctgtggaacggcaaca | |
| | aaattatcgacgagcgcctgatcacccccgacggctccatgctg | |
| | ttccgagtaaccatcaacagcctcgaggactacaaggacgacga | |
| | tgacaag | |
| NLS-HA-MCP- | cgttacataacttacggtaaatggcccgcctggctgaccgccca | 14 |
| linker-SmBi114- | acgacccccgcccattgacgtcaataatgacgtatgttcccata | |
| IRES-PCP-linker- | gtaacgccaatagggactttccattgacgtcaatgggtggagta | |
| YeLgBİT-FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgcggattctagacgggatcccgccaccgtgaccg | |
| | gctaccgcctgtttgaggagattctgtaacgtacgacgcgtgga | |
| | tccctcccccccccctaacgttactggccgaagccgcttggaat | |
| | aaggccggtgtgcgtttgtctatatgttattttccaccatattg | |
| | ccgtcttttggcaatgtgagggcccggaaacctggccctgtctt | |
| | cttgacgagcattcctaggggtctttcccctctcgccaaaggaa | |
| | tgcaaggtctgttgaatgtcgtgaaggaagcagttcctctggaa | |
| | gcttcttgaagacaaacaacgtctgtagcgaccctttgcaggca | |
| | gcggaaccccccacctggcgacaggtgcctctgcggccaaaagc | |
| | cacgtgtataagatacacctgcaaaggcggcacaaccccagtgc | |
| | cacgttgtgagttggatagttgtggaaagagtcaaatggctctc | |
| | ctcaagcgtattcaacaaggggctgaaggatgcccagaaggtac | |
| | cccattgtatgggatctgatctggggcctcggtgcacatgcttt | |
| | acatgtgtttagtcgaggttaaaaaaacgtctaggccccccgaa | |
| | ccacggggacgtggttttcctttgaaaaacacgatgataatatg | |
| | gccacagcctgcaggatgggctccaaaaccatcgttctttcggt | |
| | cggcgaggctactcgcactctgactgagatccagtccaccgcag | |
| | accgtcagatcttcgaagagaaggtcgggcctctggtgggtcgg | |
| | ctgcgcctcacggcttcgctccgtcaaaacggagccaagaccgc | |
| | gtatcgcgtcaacctaaaactggatcaggcggacgtcgttgatt | |
| | ccggacttccgaaagtgcgctacactcaggtatggtcgcacgac | |
| | gtgacaatcgttgcgaatagcaccgaggcctcgcgcaaatcgtt | |
| | gtacgatttgaccaagtccctcgtcgcgacctcgcaggtcgaag | |
| | atcttgtcgtcaaccttgtgccgctgggccgtgcggatccaccg | |
| | gtatgcatcgccaccgccgtgagcaagggcgaggagctgttcac | |
| | cggggtggtgcccatcctggtcgagctggacggcgacgtaaacg | |
| | gccacaagttcagcgtgtccggcgagggcgagggcgatgccacc | |
| | tacggcaagctgaccctgaagctgatctgcaccaccggcaagct | |
| | gcccgtgccctggcccaccctcgtgaccaccctgggctacggcc | |
| | tgcagtgcttcgcccgctaccccgaccacatgaagcagcacgac | |
| | ttcttcaagtccgccatgcccgaaggctacgtccaggagcgcac | |
| | catcttcttcaaggacgacggcaactacaagacccgcgccgagg | |
| | tgaagttcgagggcgacaccctggtgaaccgcatcgagctgaag | |
| | ggcatcgacttcaaggaggacggcaacatcctggggcacaagct | |
| | ggagtacaactacaacagccacaacgtctatatcaccgccgaca | |
| | agcagaagaacggcatcaaggccaacttcaagatccgccacaac | |
| | atcgaggacggcggcgtgcagctcgccgaccactaccagcagaa | |
| | cacccccatcggcgacggccccgtgctgctgcccgacaaccact | |
| | acctgagctaccagtccgccctgagcaaagaccccaacgagaag | |
| | cgcgatcacatggtcctgctggagttcgtgaccgccgcgatgtt | |
| | cacactcgaagatttcgttggggactgggaacagacagccgcct | |
| | acaacctggaccaagtccttgaacagggaggtgtgtccagtttg | |
| | ctgcagaatctcgccgtgtccgtaactccgatccaaaggattgt | |
| | ccggagcggtgaaaatgccctgaagatcgacatccatgtcatca | |
| | tcccgtatgaaggtctgagcgccgaccaaatggcccagatcgaa | |
| | gaggtgtttaaggtggtgtaccctgtggatgatcatcactttaa | |
| | ggtgatcctgccctatggcacactggtaatcgacggggttacgc | |
| | cgaacatgctgaactatttcggacggccgtatgaaggcatcgcc | |
| | gtgttcgacggcaaaaagatcactgtaacagggaccctgtggaa | |
| | cggcaacaaaattatcgacgagcgcctgatcacccccgacggct | |
| | ccatgctgttccgagtaaccatcaacagcctcgaggactacaag | |
| | gacgacgatgacaag | |
| NLS-HA-MCP- | cgttacataacttacggtaaatggcccgcctggctgaccgccca | 15 |
| linker-SmBIT114- | acgacccccgcccattgacgtcaataatgacgtatgttcccata | |
| IRES-PCP-linker- | gtaacgccaatagggactttccattgacgtcaatgggtggagta | |
| LumiLgBiT-FLAG | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatgggcccaaaaaagaaaagaaaagttggcta | |
| | cccctacgacgtgcccgactacgccatcgaaggccgccatatgc | |
| | tagccgttaaaatggcttctaactttactcagttcgttctcgtc | |
| | gacaatggcggaactggcgacgtgactgtcgccccaagcaactt | |
| | cgctaacgggatcgctgaatggatcagctctaactcgcgttcac | |
| | aggcttacaaagtaacctgtagcgttcgtcagagctctgcgcag | |
| | aatcgcaaatacaccatcaaagtcgaggtgcctaaaggcgcctg | |
| | gcgttcgtacttaaatatggaactaaccattccaattttcgcca | |
| | cgaattccgactgcgagcttattgttaaggcaatgcaaggtctc | |
| | ctaaaagatggaaacccgattccctcagcaatcgcagcaaactc | |
| | cggcatctacgcggattctagacgggatcccgccaccgtgaccg | |
| | gctaccgcctgtttgaggagattctgtaacgtacgacgcgtgga | |
| | tccctcccccccccctaacgttactggccgaagccgcttggaat | |
| | aaggccggtgtgcgtttgtctatatgttattttccaccatattg | |
| | ccgtcttttggcaatgtgagggcccggaaacctggccctgtctt | |
| | cttgacgagcattcctaggggtctttcccctctcgccaaaggaa | |
| | tgcaaggtctgttgaatgtcgtgaaggaagcagttcctctggaa | |
| | gcttcttgaagacaaacaacgtctgtagcgaccctttgcaggca | |
| | gcggaaccccccacctggcgacaggtgcctctgcggccaaaagc | |
| | cacgtgtataagatacacctgcaaaggcggcacaaccccagtgc | |
| | cacgttgtgagttggatagttgtggaaagagtcaaatggctctc | |
| | ctcaagcgtattcaacaaggggctgaaggatgcccagaaggtac | |
| | cccattgtatgggatctgatctggggcctcggtgcacatgcttt | |
| | acatgtgtttagtcgaggttaaaaaaacgtctaggccccccgaa | |
| | ccacggggacgtggttttcctttgaaaaacacgatgataatatg | |
| | gccacagcctgcaggatgggctccaaaaccatcgttctttcggt | |
| | cggcgaggctactcgcactctgactgagatccagtccaccgcag | |
| | accgtcagatcttcgaagagaaggtcgggcctctggtgggtcgg | |
| | ctgcgcctcacggcttcgctccgtcaaaacggagccaagaccgc | |
| | gtatcgcgtcaacctaaaactggatcaggcggacgtcgttgatt | |
| | ccggacttccgaaagtgcgctacactcaggtatggtcgcacgac | |
| | gtgacaatcgttgcgaatagcaccgaggcctcgcgcaaatcgtt | |
| | gtacgatttgaccaagtccctcgtcgcgacctcgcaggtcgaag | |
| | atcttgtcgtcaaccttgtgccgctgggccgtgcggatccaccg | |
| | gtatgcatcgccaccgccgtgagcaagggcgaggcagtgatcaa | |
| | ggagttcatgcggttcaaggtgcacatggagggctccatgaacg | |
| | gccacgagttcgagatcgagggcgagggcgagggccgcccctac | |
| | gagggcacccagaccgccaagctgaaggtgaccaagggtggccc | |
| | cctgcccttctcctgggacatcctgtcccctcagttcatgtacg | |
| | gctccagggccttcatcaagcaccccgccgacatccccgactac | |
| | tataagcagtccttccccgagggcttcaagtgggagcgcgtgat | |
| | gaacttcgaggacggcggcgccgtgaccgtgacccaggacacct | |
| | ccctggaggacggcaccctgatctacaaggtgaagctccgcggc | |
| | accaacttccctcctgacggccccgtaatgcagaagaagacaat | |
| | gggctgggaagcgtccaccgagcggttgtaccccgaggacggcg | |
| | tgctgaagggcgacattaagatggccctgcgcctgaaggacggc | |
| | ggccgctacctggcggacttcaagaccacctacaaggccaagaa | |
| | gcccgtgcagatgcccggcgcctacaacgtcgaccgcaagttgg | |
| | acatcacctcccacaacgaggactacaccgtggtggaacagtac | |
| | gaacgctccgagggccgccactccaccggaaagttcacactcga | |
| | agatttcgttggggactgggaacagacagccgcctacaacctgg | |
| | accaagtccttgaacagggaggtgtgtccagtttgctgcagaat | |
| | ctcgccgtgtccgtaactccgatccaaaggattgtccggagcgg | |
| | tgaaaatgccctgaagatcgacatccatgtcatcatcccgtatg | |
| | aaggtctgagcgccgaccaaatggcccagatcgaagaggtgttt | |
| | aaggtggtgtaccctgtggatgatcatcactttaaggtgatcct | |
| | gccctatggcacactggtaatcgacggggttacgccgaacatgc | |
| | tgaactatttcggacggccgtatgaaggcatcgccgtgttcgac | |
| | ggcaaaaagatcactgtaacagggaccctgtggaacggcaacaa | |
| | aattatcgacgagcgcctgatcacccccgacggctccatgctgt | |
| | tccgagtaaccatcaacagcctcgaggactacaaggacgacgat | |
| | gacaag | |
| NLS-HA-MCP- | cgttacataacttacggtaaatggcccgcctggctgaccgccca | 16 |
| FlucN-IRES-PCP | acgacccccgcccattgacgtcaataatgacgtatgttcccata | |
| -FlucC-FLAG | gtaacgccaatagggactttccattgacgtcaatgggtggagta | |
| | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctcgtttagtgaaccgtcagat | |
| | cgcctggagacgccatccacgctgttttgacctccatagaagac | |
| | accgactctagagccaccatgggcccaaaaaagaaaagaaaagt | |
| | tggctacccctacgacgtgcccgactacgccatcgaaggccgcc | |
| | atatgctagccgttaaaatggcttctaactttactcagttcgtt | |
| | ctcgtcgacaatggcggaactggcgacgtgactgtcgccccaag | |
| | caacttcgctaacgggatcgctgaatggatcagctctaactcgc | |
| | gttcacaggcttacaaagtaacctgtagcgttcgtcagagctct | |
| | gcgcagaatcgcaaatacaccatcaaagtcgaggtgcctaaagg | |
| | cgcctggcgttcgtacttaaatatggaactaaccattccaattt | |
| | tcgccacgaattccgactgcgagcttattgttaaggcaatgcaa | |
| | ggtctcctaaaagatggaaacccgattccctcagcaatcgcagc | |
| | aaactccggcatctacgcggattctagacgggatcccgccaccg | |
| | aagacgccaaaaacataaagaaaggcccggcgccattctatccg | |
| | ctggaagatggaaccgctggagagcaactgcataaggctatgaa | |
| | gagatacgccctggttcctggaacaattgcttttacagatgcac | |
| | atatcgaggtggacatcacttacgctgagtacttcgaaatgtcc | |
| | gttcggttggcagaagctatgaaacgatatgggctgaatacaaa | |
| | tcacagaatcgtcgtatgcagtgaaaactctcttcaattcttta | |
| | tgccggtgttgggcgcgttatttatcggagttgcagttgcgccc | |
| | gcgaacgacatttataatgaacgtgaattgctcaacagtatggg | |
| | catttcgcagcctaccgtggtgttcgtttccaaaaaggggttgc | |
| | aaaaaattttgaacgtgcaaaaaaagctcccaatcatccaaaaa | |
| | attattatcatggattctaaaacggattaccagggatttcagtc | |
| | gatgtacacgttcgtcacatctcatctacctcccggttttaatg | |
| | aatacgattttgtgccagagtccttcgatagggacaagacaatt | |
| | gcactgatcatgaactcctctggatctactggtctgcctaaagg | |
| | tgtcgctctgcctcatagaactgcctgcgtgagattctcgcatg | |
| | ccagagatcctatttttggcaatcaaatcattccggatactgcg | |
| | attttaagtgttgttccattccatcacggttttggaatgtttac | |
| | tacactcggatatttgatatgtggatttcgagtcgtcttaatgt | |
| | atagatttgaagaagagctgtttctgaggagccttcaggattac | |
| | aagattcaaagtgcgctgctggtgccaaccctattctccttctt | |
| | cgccaaaagcactctgattgacaaatacgatttatctaatttac | |
| | acgaaattgcttctggtggcgctcccctctctaaggaagtcggg | |
| | gaagcggttgccaagaggttccatctgccaggtatcaggcaagg | |
| | atatgggctcactgagactacatcagctattctgattacacccg | |
| | agggggatgataaaccgggcgcggtcggtaaagttgttccattt | |
| | tttgaagcgaaggttgtggatctggataccgggaaaacgctggg | |
| | cgttaatcaaagaggcgaactgtgtgtgagaggtcctatgatta | |
| | tgtcctaacgtacgacgcgtggatccctcccccccccctaacgt | |
| | tactggccgaagccgcttggaataaggccggtgtgcgtttgtct | |
| | atatgttattttccaccatattgccgtcttttggcaatgtgagg | |
| | gcccggaaacctggccctgtcttcttgacgagcattcctagggg | |
| | tctttcccctctcgccaaaggaatgcaaggtctgttgaatgtcg | |
| | tgaaggaagcagttcctctggaagcttcttgaagacaaacaacg | |
| | tctgtagcgaccctttgcaggcagcggaaccccccacctggcga | |
| | caggtgcctctgcggccaaaagccacgtgtataagatacacctg | |
| | caaaggcggcacaaccccagtgccacgttgtgagttggatagtt | |
| | gtggaaagagtcaaatggctctcctcaagcgtattcaacaaggg | |
| | gctgaaggatgcccagaaggtaccccattgtatgggatctgatc | |
| | tggggcctcggtgcacatgctttacatgtgtttagtcgaggtta | |
| | aaaaaacgtctaggccccccgaaccacggggacgtggttttcct | |
| | ttgaaaaacacgatgataatatggccacagcctgcaggatgggc | |
| | tccaaaaccatcgttctttcggtcggcgaggctactcgcactct | |
| | gactgagatccagtccaccgcagaccgtcagatcttcgaagaga | |
| | aggtcgggcctctggtgggtcggctgcgcctcacggcttcgctc | |
| | cgtcaaaacggagccaagaccgcgtatcgcgtcaacctaaaact | |
| | ggatcaggcggacgtcgttgattccggacttccgaaagtgcgct | |
| | acactcaggtatggtcgcacgacgtgacaatcgttgcgaatagc | |
| | accgaggcctcgcgcaaatcgttgtacgatttgaccaagtccct | |
| | cgtcgcgacctcgcaggtcgaagatcttgtcgtcaaccttgtgc | |
| | cgctgggccgtgcggatccaccggtatgcatcgccaccgcccct | |
| | atgattatgtccggttatgtaaacaatccggaagcgaccaacgc | |
| | cttgattgacaaggatggatggctacattctggagacatagctt | |
| | actgggacgaagacgaacacttcttcatcgttgaccgcctgaag | |
| | tctctgattaagtacaaaggctatcaggtggctcccgctgaatt | |
| | ggaatccatcttgctccaacaccccaacatcttcgacgcaggtg | |
| | tcgcaggtcttcccgacgatgacgccggtgaacttcccgccgcc | |
| | gttgttgttttggagcacggaaagacgatgacggaaaaagagat | |
| | cgtggattacgtcgccagtcaagtaacaaccgcgaaaaagttgc | |
| | gcggaggagttgtgtttgtggacgaagtaccgaaaggtcttacc | |
| | ggaaaactcgacgcaagaaaaatcagagagatcctcataaaggc | |
| | caagaagggcggaaagatcgccgtgctcgaggactacaaggacg | |
| | acgatgacaag | |
| BFP-MS2-3-PP7 | cgttacataacttacggtaaatggcccgcctggctgaccgccca | 17 |
| | acgacccccgcccattgacgtcaataatgacgtatgttcccata | |
| | gtaacgccaatagggactttccattgacgtcaatgggtggagta | |
| | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttcgccaccatgagcgagctgattaaggagaaca | |
| | tgcacatgaagctgtacatggagggcaccgtggacaaccatcac | |
| | ttcaagtgcacatccgagggcgaaggcaagccctacgagggcac | |
| | ccagaccatgagaatcaaggtggtcgagggcggccctctcccct | |
| | tcgccttcgacatcctggctactagcttcctctacggcagcaag | |
| | accttcatcaaccacacccagggcatccccgacttcttcaagca | |
| | gtccttccctgagggcttcacatgggagagagtcaccacatacg | |
| | aagacgggggcgtgctgaccgctacccaggacaccagcctccag | |
| | gacggctgcctcatctacaacgtcaagatcagaggggtgaactt | |
| | cacatccaacggccctgtgatgcagaagaaaacactcggctggg | |
| | aggccttcaccgagacgctgtaccccgctgacggcggcctggaa | |
| | ggcagaaacgacatggccctgaagctcgtgggcgggagccatct | |
| | gatcgcaaacatcaagaccacatatagatccaagaaacccgcta | |
| | agaacctcaagatgcctggcgtctactatgtggactacagactg | |
| | gaaagaatcaaggaggccaacaacgagacctacgtcgagcagca | |
| | cgaggtggcagtggccagatactgcgacctccctagcaaactgg | |
| | ggcacaagcttaattaagaattctgcagatatccatcacactgg | |
| | cggccgcgggagacgagcatcagccgtcgagagcagacgatatg | |
| | gcgtcgctcggcaattgaaaaaacgccctccc | |
| BFP-MS2-3-PP7 | cgttacataacttacggtaaatggcccgcctggctgaccgccca | 18 |
| (mut) | acgacccccgcccattgacgtcaataatgacgtatgttcccata | |
| | gtaacgccaatagggactttccattgacgtcaatgggtggagta | |
| | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttcgccaccatgagcgagctgattaaggagaaca | |
| | tgcacatgaagctgtacatggagggcaccgtggacaaccatcac | |
| | ttcaagtgcacatccgagggcgaaggcaagccctacgagggcac | |
| | ccagaccatgagaatcaaggtggtcgagggcggccctctcccct | |
| | tcgccttcgacatcctggctactagcttcctctacggcagcaag | |
| | accttcatcaaccacacccagggcatccccgacttcttcaagca | |
| | gtccttccctgagggcttcacatgggagagagtcaccacatacg | |
| | aagacgggggcgtgctgaccgctacccaggacaccagcctccag | |
| | gacggctgcctcatctacaacgtcaagatcagaggggtgaactt | |
| | cacatccaacggccctgtgatgcagaagaaaacactcggctggg | |
| | aggccttcaccgagacgctgtaccccgctgacggcggcctggaa | |
| | ggcagaaacgacatggccctgaagctcgtgggcgggagccatct | |
| | gatcgcaaacatcaagaccacatatagatccaagaaacccgcta | |
| | agaacctcaagatgcctggcgtctactatgtggactacagactg | |
| | gaaagaatcaaggaggccaacaacgagacctacgtcgagcagca | |
| | cgaggtggcagtggccagatactgcgacctccctagcaaactgg | |
| | ggcacaagcttaattaagaattctgcagatatccatcacactgg | |
| | cggccgcgggagacgagcatcagccgtcgagagcgacgttatgg | |
| | cgtcgctcggcaattgaaaaaacgccctccc | |
| Staygold-M-3-P | cgttacataacttacggtaaatggcccgcctggctgaccgccca | 19 |
| | acgacccccgcccattgacgtcaataatgacgtatgttcccata | |
| | gtaacgccaatagggactttccattgacgtcaatgggtggagta | |
| | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttgcggccgccaccatgctgctgcccgtccccct | |
| | gctgctgggcctgctgggcgccgccgcggatccgatggtgagca | |
| | agggcgaggagctgtttacaggcgtgacccccttcaagttccag | |
| | ctgaagggcaccatcaacggcaagagcttcaccgtggaaggcga | |
| | gggcgagggcaatagccacgagggcagccacaaaggcaagtacg | |
| | tgtgcaccagcggcaaactgccaatgtcttgggccgccctggga | |
| | actagcttcggctatggcatgaagtactacaccaagtaccccag | |
| | cggcctgaagaactggttccacgaggtgatgcccgagggcttca | |
| | cctacgacagacacatccagtacaagggcgacggcagcatccac | |
| | gccaagcaccagcacttcatgaagaacggcacctaccacaacat | |
| | cgtggagttcaccggccaggacttcaaggagaacagccccgtgc | |
| | tgaccggcgacatgaacgtgagcctgcccaacgaggtgcagcac | |
| | atccccagagatgacggcgtggagtgcccagtgaccctgctgta | |
| | ccctctgctgagcgacaagagcaagtgcgtggaggcctaccaga | |
| | acaccatcatcaagcccctgcacaatcagccagcccccgatgtg | |
| | ccataccactggatcagaaagcagtacacccagagcaaggacga | |
| | caccgaggagagagaccacatcatccagagcgagaccctggagg | |
| | cccacctgccatggcacgagccttctgcttctgccgtgaaggat | |
| | gaactgtaagaattctgcagatatccatcacactggcggccgcg | |
| | ggagacgagcatcagccgtcgagagcagacgatatggcgtcgct | |
| | cggcaattgaaaaaacgccctccc | |
| Staygold | cgttacataacttacggtaaatggcccgcctggctgaccgccca | 20 |
| | acgacccccgcccattgacgtcaataatgacgtatgttcccata | |
| | gtaacgccaatagggactttccattgacgtcaatgggtggagta | |
| | tttacggtaaactgcccacttggcagtacatcaagtgtatcata | |
| | tgccaagtacgccccctattgacgtcaatgacggtaaatggccc | |
| | gcctggcattatgcccagtacatgaccttatgggactttcctac | |
| | ttggcagtacatctacgtattagtcatcgctattaccatggtga | |
| | tgcggttttggcagtacatcaatgggcgtggatagcggtttgac | |
| | tcacggggatttccaagtctccaccccattgacgtcaatgggag | |
| | tttgttttggcaccaaaatcaacgggactttccaaaatgtcgta | |
| | acaactccgccccattgacgcaaatgggcggtaggcgtgtacgg | |
| | tgggaggtctatataagcagagctctctggctaactagagaacc | |
| | cactgcttactggcttatcgaaattaatacgactcactataggg | |
| | agacccaagcttatggcgtctactcccttcaagttccagctgaa | |
| | gggcaccatcaacggcaagagcttcaccgtggaaggcgagggcg | |
| | agggcaatagccacgagggcagccacaaaggcaagtacgtgtgc | |
| | accagcggcaaactgccaatgtcttgggccgccctgggaactag | |
| | cttcggctatggcatgaagtactacaccaagtaccccagcggcc | |
| | tgaagaactggttccacgaggtgatgcccgagggcttcacctac | |
| | gacagacacatccagtacaagggcgacggcagcatccacgccaa | |
| | gcaccagcacttcatgaagaacggcacctaccacaacatcgtgg | |
| | agttcaccggccaggacttcaaggagaacagccccgtgctgacc | |
| | ggcgacatgaacgtgagcctgcccaacgaggtgcagcacatccc | |
| | cagagatgacggcgtggagtgcccagtgaccctgctgtaccctc | |
| | tgctgagcgacaagagcaagtgcgtggaggcctaccagaacacc | |
| | atcatcaagcccctgcacaatcagccagcccccgatgtgccata | |
| | ccactggatcagaaagcagtacacccagagcaaggacgacaccg | |
| | aggagagagaccacatcatccagagcgagaccctggaggcccac | |
| | ctg | |

RNA bait DNA sequences (T7 promoter)

| | | |
|---|---|---|
| Flexible | gtaatacgactcactataGGCACGAGCATCAGCCGTGCCTCCAG | 21 |
| | GTCGAATCTTCAAACGAGCAGACGATATGGCGTCGCTC | |
| | G | |
| MS2 | gtaatacgactcactataGGCACGAGCATCAGCCGTGCC | 22 |
| PP7 | gtaatacgactcactataGGAGCAGACGATATGGCGTCGCTCG | 23 |
| MS2-3-PP7Mut_Ni | gtaatacgactcactataGGGAGACGAGCATCAGCCGTCGAGAG | 24 |
| | CGACGTTATGGCGTCGCTCGGCAATTGAAAAAACGCCC | |
| | TCCC | |
| MS2-0-PP7-Ni | gtaatacgactcactataGGGACACGAGCATCAGCCGTGGAGCA | 25 |
| | GACGATATGGCGTCGCTCGCGAATTGAAAAAACCGCTC | |
| | CC | |
| MS2-1-PP7-Ni | gtaatacgactcactataGGGACGCGAGCATCAGCCGCGAGAG | 26 |
| | CAGACGATATGGCGTCGCTCGCGAATTGAAAAAACCGC | |
| | TCCC | |
| MS2-3-PP7-Ni | gtaatacgactcactataGGGAGACGAGCATCAGCCGTCGAGAG | 27 |
| | CAGACGATATGGCGTCGCTCGGCAATTGAAAAAACGCC | |
| | CTCCC | |
| MS2-5-PP7-Ni | gtaatacgactcactataGGGAGACGAGCATCAGCCGTCGGAGA | 28 |
| | GCAGACGATATGGCGTCGCTCGGCAATTGAAAAAACGC | |
| | CCCTCCC | |
| MS2-7-PP7-Ni | gtaatacgactcactataGGGAGACGAGCATCAGCCGTCCGGAG | 29 |
| | AGCAGACGATATGGCGTCGCTCGGCAATTGAAAAAACG | |
| | CCCCGTCCC | |
| MS2-9-PP7-Ni | gtaatacgactcactataGGGAGACGAGCATCAGCCGTCCGAGA | 30 |
| | GAGCAGACGATATGGCGTCGCTCGGCAATTGAAAAAAC | |
| | GCCCTCGTCCC | |
| MS2-11-PP7-Ni | gtaatacgactcactataGGGAGACGAGCATCAGCCGTCCGACG | 31 |
| | AGAGCAGACGATATGGCGTCGCTCGGCAATTGAAAAAA | |
| | CGCCCGTCGTCCC | |
| MS2-13-Ni-PP7 | gtaatacgactcactataGGGAGACGAGCATCAGCCGTCCTGAG | 32 |
| | CGGCAATTGAAAAAACGCCGAGCAGACGATATGGCGTC | |
| | GCTCGCTCAGTCCC | |
| MS2-21-PP7-Ni | gtaatacgactcactataGGGAGACGAGCATCAGCCGTCTCGAC | 33 |
| | GAGCAAGAGCAGACGATATGGCGTCGCTCGGCAATTGA | |
| | AAAAACGCCTGCTCGTCGATCCC | |
| 7-MS2-7-PP7-Ni | gtaatacgactcactataGGGACGACGAGCATCAGCCGTCGCG | 34 |
| | GAGAGCAGACGATATGGCGTCGCTCGGCAATTGAAAAA | |
| | ACGCCCCGTCCC | |
| 8-MS2-7-PP7-Ni | gtaatacgactcactataGGGACGACTGAGCATCAGCCAGTCGC | 35 |
| | GGAGAGCAGACGATATGGCGTCGCTCGGCAATTGAAAA | |
| | AACGCCCCGTCCC | |
| 7-MS2-3-PP7-Ni | gtaatacgactcactataGGGACGACGAGCATCAGCCGTCGGAG | 36 |
| | AGCAGACGATATGGCGTCGCTCGGCAATTGAAAAAACG | |
| | CCCTCCC | |
| 8-MS2-3-PP7-Ni | gtaatacgactcactataGGGACGACTGAGCATCAGCCAGTCGG | 37 |
| | AGAGCAGACGATATGGCGTCGCTCGGCAATTGAAAAAA | |
| | CGCCCTCCC | |
| 6-MS2-3-6-PP7-Ni | gtaatacgactcactataGGGAGACGAGCATCAGCCGTCGAGCA | 38 |
| | GACGATATGGCGTCGCGGCAATTGAAAAAACGCCCTCC | |
| | C | |
| 6-MS2-3-9-PP7-Ni | gtaatacgactcactataGGGAGACGAGCATCAGCCGTCGAGAG | 39 |
| | CGAGACGATATGGCGTCCGCTCGGCAATTGAAAAAACG | |
| | CCCTCCC | |
| 6-MS2-3-10-PP7-Ni | gtaatacgactcactataGGGAGACGAGCATCAGCCGTCGAGAG | 40 |
| | CGCAGACGATATGGCGTCGCGCTCGGCAATTGAAAAAA | |
| | CGCCCTCCC | |
| PP7-9-MS2-Ni | gtaatacgactcactataGGGAGAGCAGACGATATGGCGTCGCT | 41 |
| | CCCAGACACGAGCATCAGCCGTGGGCAATTGAAAAAAC | |
| | GCCCTGGTCCC | |

General cell culture methods: HEK293T cells (HEK, ATCC) and stable cells lines derived from HEK293T cells were cultured in complete media: DMEM (Corning) containing 10% (v/v) fetal bovine serum (FBS, Life Technologies), penicillin (100 U/mL), and streptomycin (100 μg/mL, Gibco). Cell lines stably expressing RNA lantern and linker designs were generated via lentiviral transduction. Transduced cells were further cultured with puromycin (20 μg/mL) to preserve gene incorporation. Cells were incubated at 37° C. in a 5% C02 humidified chamber. Cells were serially passaged using trypsin (0.25% in HBSS, Gibco).

Protein expression and purification: LgBiT was encoded in a pCold vector. LgBiT was expressed in E. coli BL21 cells grown in LB medium (1 L). Expression was induced at an optical density (OD600) of ˜0.6 by addition of 0.5 mM isopropyl-43-D-thiogalactopyranoside (IPTG), followed by incubation at 37° C. for 4 h. Cells were harvested by centrifugation (4,000×g, 10 min, 4° C.). Cells were then resuspended in lysis buffer (30 mL, 50 mM Tris HCl, 150 mM NaCl, 0.5% Tween-20, 1 mM phenylmethylsulfonyl fluoride [PMSF], pH 7.4). Cells were sonicated (Qsonica) at 40% amplitude, at 2 sec on 2 sec off intervals for 15 min. Cell debris was removed through centrifugation (10,000×g, 1 h, 4° C.). Proteins were purified by Ni-NTA affinity chromatography. The column was washed with wash buffer (20 mM imidazole, 50 mM NaPO₄, pH 7.4). Protein was eluted from column with elution buffer (200 mM imidazole, 50 mM NaPO₄, pH 7.4). Protein was dialyzed overnight at 4° C. into phosphate buffer (50 mM NaPO₄, pH 7.4). Protein was concentrated to ˜500 μL using Amicon Ultra-15 Centrifugal Filter Units (Merck Millipore MWCO 3 kDa). The concentration of proteins was determined using JASCO V730 UV-vis spectrophotometer at 280 nm. SDS-PAGE was also performed to verify purity, and gels were stained with Coomassie R-250.

In vitro transcription: RNA was transcribed in vitro in a buffer containing 50 mM Tris-HCl pH 8, 2 mM spermidine, 0.01% Triton X-100, 2 mM rNTPs (each), 20 mM MgCl₂, 10 mM dithiothreitol (DTT), and recombinant T7 RNA polymerase. Transcription reactions were quenched with 30 mM EDTA and subsequently ethanol-precipitated with coprecipitant (Invitrogen GlycoBlue, AM9516) prior to purification via denaturing PAGE. Purified RNAs were eluted from gel pieces into 300 mM KCl and 0.1 mM EDTA for 3 h. Eluted RNAs were ethanol-precipitated with coprecipitant, and pellets were washed with 70% ethanol, dried, and resuspended in 10 mM Tris-HCl pH 7.5, 0.1 mM EDTA, and 0.001% Triton X-100 (TET). RNA concentrations were determined by UV-vis absorption spectroscopy (Thermo Scientific NanoDrop 2000, ND-2000).

In vitro transcription/translation (IVTT) in rabbit reticulocyte lysate: A rabbit reticulocyte lysate (RRL) in vitro translation kit (Promega, L4960) was coupled to in vitro transcription with the supplementation of MgCl2, rNTPs, and T7 RNA polymerase. Plasmid DNAs were 3′ linearized for run-off transcription. RNA scaffolds were prepared via primer elongation of synthetic DNA oligos (Integrated DNA Technologies). Linearized plasmid DNAs and RNA bait DNAs were kit purified prior to IVTT (DNA Clean and Concentrator; Zymo Research, D4004). All DNAs were eluted into Tris-EDTA (TE; 10 mM Tris-HCl and 0.1 mM EDTA, pH 7.5) and controls were supplemented with an equal volume of TE buffer.

IVTT reactions were prepared on ice with 70% v/v lysate, 0.02 mM amino acid mix, rNTPs (0.35 mM GTP and 0.15 mM each ATP/CTP/UTP), 1 mM MgCl2, linearized plasmid DNA, RNA bait template DNA, 2 U/10 μL RNase inhibitor (Invitrogen, AM2694), and 0.5 μL/10 μL reaction of recombinant T7 RNA polymerase (house preparation). NanoBiT experiments were set up with 10 μL volumes and 1 nM of linearized plasmid DNA. Split firefly luciferase experiments were performed with 25 μL volumes and 3 nM of linearized plasmid DNA. RNA bait DNA templates were varied at specified ratios. IVTT reactions were incubated at 34° C. for 10 min and 30° C. for 140 min.

Luciferin substrates were supplied prior to imaging. For split NanoBiT RNA lantern experiments, furimazine was added to each reaction at 20 μM, and samples were incubated at room temperature for 5 min before imaging. For split firefly luciferase RNA lantern experiments, D-luciferin (100 μM) and ATP (1 mM) were added to each reaction. Samples were then incubated at room temperature for 5 min before image acquisition. Plates were imaged in a dark, light-proof chamber using an IVIS Lumina (PerkinElmer) CCD camera chilled to −90° C. The stage was kept at 37° C. during imaging and the camera was controlled using Living Image software. Exposure times were set to 1 min and binning levels were set to medium. Regions of interest were selected for quantification and total flux values were analyzed using Living Image software. All data was exported to Microsoft Excel or Prism (GraphPad) for further analysis.

Reconstitution kinetics: Three reticulocyte lysate IVTT reactions (10 μL) were set up as above. Reactions were incubated at 34° C. for 10 min and 30° C. for 140 min. Furimazine (20 μM) was added to each reaction, and then samples were placed in a black, clear bottom 384-well plate. Purified M-3-P RNA (1 nM), M-3-P DNA (1 nM), or GFP-M-3-P DNA (1 nM) was added individually to each reaction. The reactions were immediately imaged using an Andor iXon Ultra 888 EMCCD camera equipped with an F0.95 lens, with an EM gain of 1000. Images were acquired (30 s acquisition) over 60 min. Images were processed with ImageJ and densitometry was used to measure mean intensity values over time. All data was exported to Microsoft Excel for analysis then plotted using GraphPad Prism.

In cellulo linker optimization: Stable cell lines expressing variant RNA lanterns were plated in clear 12-well plates and incubated overnight. Cell lines were then transfected with 1 μL Lipofectamine 3000 (Invitrogen, L3000001), 2 μL P3000 Reagent (Invitrogen, L3000001), and 500 ng of the BFP-M-3-P plasmid. After 24 h incubation, cells were lifted and counted with a Countess II (Invitrogen). 50,000 transfected or non-transfected cells were plated in triplicate into black 96-well plates (Greiner Bio-One). Furimazine (20 μM) was added to each sample. Plates were imaged in a dark, light-proof chamber using an IVIS Lumina (PerkinElmer) CCD camera chilled to −90° C. The stage was kept at 37° C. during imaging and the camera was controlled using Living Image software. Exposure times were set to 1 min and binning levels were set to medium. Regions of interest were selected for quantification and total flux values were analyzed using Living Image software. All data was exported to Microsoft Excel or Prism (GraphPad) for further analysis. Remaining cells were analyzed on a Novocyte 3000 flow cytometer (ACEA BioSciences) for BFP expression.

General flow cytometry methods: Cells were treated with trypsin for 5 min at 37° C. and then neutralized with complete media. Cells were transferred to Eppendorf tubes and pelleted (500×g, 5 min) using a tabletop centrifuge (Thermo Fisher Sorvall Legend Micro 17). The resulting supernatants were discarded, and cells were washed with PBS (2×400 μL). Cells were analyzed for XFP expression on a Novocyte 3000 flow cytometer. Live cells were gated using FSC/SSC settings, and singlet cells were further gated (see below). For each sample, 10,000 events were collected on the “singlet cell” gate.

HEK293T lysate preparation: HEK293T cells stably expressing RNA lanterns (˜10⁶) were pelleted at 500×g for 10 min at 4° C. and washed three times with binding buffer (140 mM KCl, 10 mM NaCl, 20 mM Tris-HCl pH 7.5, and 5 mM MgCl₂). Cells were pelleted during each washing step at 500×g (1 min, 4° C.) After the final wash, the cell pellet was resuspended in 600 μL binding buffer supplemented with 1% Tween-80, 1× protease inhibitor cocktail (Roche, 11697498001), and 5 μg RNase inhibitor. Cells were sonicated on ice (5 cycles of 10 s on, 10 s off; Branson CPX2800). Cell debris was then removed by centrifugation (12,000×g, 4° C., 15 min). The resulting supernatant was aliquoted and stored at −80° C. for future use as 2× lysate stocks.

RNA titration assay: Purified RNAs (10× stocks in TET buffer) were serially diluted. Binding reactions contained 12.5 μL of 2× cell lysate, 10 μL of 1× binding buffer, and 2.5 μL of 10×RNA. Reactions were incubated at room temperature on an orbital shaker for 30 minutes. After incubation, 1 μL of a 200 μM furimazine stock (Promega, Nano-Glo substrate) in 1× binding buffer was added to each reaction and then plated in a black, clear bottom 384-well plate and imaged with an Andor iXon Ultra 888 EMCCD camera (Oxford Instruments) equipped with an F 0.95 lens (Schneider) with an EM gain of 500. Images were acquired (5 s acquisition) over 10 min. Images were Z-stacked in ImageJ and densitometry was used to measure peak intensity values.

Ni-NTA pull-down: Triplicate reactions of selected RNA concentrations above, within, and below the curve (as determined by RNA titration assay) were combined (45 μL/each). Charged Ni-NTA agarose beads (˜45 μL; Thermo Fisher, 88221) were washed three times in 90 μL 1× binding buffer in a spin-column (Corning, 8160) at 3000×g for 30 s. The combined binding reactions were then incubated on the Ni-NTA agarose at room temperature for 30 min on an orbital shaker. After incubation, the columns were spun at 3000×g for 30 s and the flowthrough was collected. Four 5-min washes were performed with a 45 μL 1× binding buffer at room temperature on an orbital shaker. After each incubation, the columns were spun, and the flowthroughs were collected (washes 1-4). Columns were eluted three times with 1× binding buffer supplemented with 25 mM EDTA (20 mM final) and incubated for 5 min on an orbital shaker. Elution fractions were collected. Furimazine was added to the fractions (20 μM final) and the solutions were placed in a black, clear bottom 384-well plate. Images were acquired using an Andor iXon Ultra 888 EMCCD camera equipped with an F 0.95 lens, with an EM gain of 500. Images were acquired (10 s acquisitions) over 15 min. Final images were Z-stacked, and densitometry (ImageJ) was used to determine fold change over background and no RNA controls. Final images were overlaid with brightfield photos. Plots were produced using GraphPad Prism.

HA and FLAG pull-down: Reticulocyte lysate IVTT reactions (67.5 μL) were set up as previously described. To each reaction 100 nM M-3-P RNA (7.5 μL) or 1× binding buffer (7.5 μL) was added prior to incubation. Reactions were incubated at 34° C. for 10 min and 30° C. for 140 min. Pierce magnetic anti-HA beads (45 μL, ThermoFisher) and anti-FLAG beads (1.5 μL, ThermoFisher) were normalized for binding capacity. Two sets of each bead were washed three times in 1× binding buffer ten-times their initial volume (450 μL and 15 μL, respectively; 5 min with agitation). To each tube, 75 μL of IVTT reactions were added and incubated with agitation for 60 min at room temperature. The supernatant was removed and stored separately. After incubation, beads were washed three times in 1× binding buffer (75 μL, 5 min with agitation) and each wash was stored. Beads were resuspended in 30 μL 1× binding buffer and 20 μM furimazine, then plated in triplicate in a black, clear bottom 384-well plate and imaged with an Andor iXon Ultra 888 EMCCD camera equipped with an F 0.95 lens, with an EM gain of 100. Images were acquired (5 s acquisition) over 15 min. Images were Z-stacked in ImageJ and densitometry was used to measure peak intensity values.

Complex isolation and quantification: HEK293T cells (5×10⁵) expressing RNA lanterns were added to 12-well plates, and then transiently transfected 24 h later with DNA (0-1000 ng) encoding GFP-M-3-P (Lipofectamine 3000). Cells were lifted 48-h post-transfection, washed, and counted. Cells (5×10⁴) were then analyzed for luminescence intensity using a luminometer (Tecan). Remaining cells were then resuspended in ˜300 μL of PBS and divided evenly into two separate 1.5 mL centrifuge tubes. Cells were pelleted at 500×g for 10 min (4° C.), and the media was removed. The pellets were stored at −80° C. for RNA analysis.

Ni-NTA pull-down lysate preparation: Frozen cell pellets from above were thawed on ice, then resuspended in 100 μL of 1× binding buffer (140 mM KCl, 10 mM NaCl, 20 mM Tris-HCL pH 7.5, and 5 mM MgCl₂) supplemented with 1% Tween-80, 1× protease inhibitor cocktail (Roche, 11697498001), 20 U of SUPERase⋅In™ RNase Inhibitor (Invitrogen), and 20 μg/mL puromycin. Cells were lysed using a 30 G syringe needle (10 plunges) and the cell debris was pelleted by centrifugation (12,000×g, 4° C., 15 min). The resulting supernatant was discarded and the pellet was resuspended in 100 μL of 1× binding buffer supplemented with 1% Tween-80, 1× protease inhibitor cocktail, and 20 U of SUPERase⋅In™ RNase Inhibitor. The lysate was clarified by centrifugation (3000×g, 30 s) on a Spin-X column and stored on ice prior to use.

Ni-NTA pulldown: Furimazine was added to the lysate above (20 μM), and samples were placed in a black-bottom, 96-well plate. Images were acquired using an Andor iXon Ultra 888 EMCCD camera equipped with an F0.95 lens, with an EM gain of 1000. Images were acquired (30 s acquisitions) over 5 min. Final images were Z-stacked, and densitometry (ImageJ) was used to measure peak intensity values.

HisPur™ Ni-NTA magnetic beads (5 μL) per condition were washed three times with 1× binding buffer supplemented with 0.1% Tween-80 (50 μL; 5 min with agitation). Prior imaged lysate was added to each tube, and samples were incubated with agitation for 5 min at RT. The flow through was removed and stored separately on ice. Beads were washed three times in 1× binding buffer supplemented with 0.1% Tween-80 (100 μL, 10 s with agitation) and each wash was stored on ice. Beads were resuspended in 1× binding buffer supplemented with 0.1% Tween-80 (100 μL). Furimazine was added to the flow through, washes, and beads (20 μM), and samples were then plated in a black-bottom, 96-well plate. Images were acquired using an Andor iXon Ultra 888 EMCCD camera equipped with an F0.95 lens, with an EM gain of 1000. Images were acquired (30 s acquisitions) over 5 min. Final images were Z-stacked, and densitometry (ImageJ) was used to determine fold change of RNA lanterns captured on the beads over background and no mRNA control.

Phenol-chloroform extraction: GFP-M-3-P mRNA pulled down on the Ni-NTA beads was transferred to a 1.5 mL centrifuge tube (100 μL). Equal volumes of phenol:chloroform:isoamyl alcohol (25:24:1) were added, and samples were vortexed for 30 s. Samples were then centrifuged at 14,000×g for 3 min, and the aqueous phase was collected and transferred to a new tube. The extraction was repeated an additional two times for a total of three extractions. RNA was ethanol-precipitated overnight at −80° C. with 1 μL GlycoBlue co-precipitant (ThermoFisher) and 100 mM KCl. The pellets were washed twice with cold 70% ethanol, dried, and stored in −80° C. for future use.

TRIzol extraction: Frozen HEK293T cell pellets were thawed on ice and resuspended in TRIzol Reagent (500 μL, Invitrogen). The RNA was isolated following the manufacturer's instructions. A portion of the aqueous phase (290 μL) for each condition was then precipitated with isopropanol (500 μL, RT). Samples were pelleted by centrifugation at 12,000×g (20 min, 4° C.), washed twice with cold 70% ethanol, dried, and stored in −80° C. for future use.

RT-qPCR: RNA pellets from both the phenol-chloroform and TRIzol extractions were treated with DNase I (New England Biolabs) following the manufacturer's protocol, then column purified using a Zymo Research RNA Clean and Concentrator kit. The RNA was reverse transcribed using a reverse primer for M-3-P and Bst 3.0 DNA polymerase (New England Biolabs)/ProtoScript II reverse transcriptase (New England Biolabs). Quantitative RT-PCR was performed on a Bio-Rad CFX Connect system using Luna® Universal qPCR Master Mix (New England Biolabs). Designed primers were acquired from Integrated DNA Technologies and are provided in Table S1/List S1. The initial DNA quantity of each sample was determined by interpolation of the quantification cycle (Cq) from a standard curve using the GFP-M-3-P DNA and primer set.

Bioluminescence microscopy: HEK293T cells (5×105) stably expressing RNA lanterns were plated in 8-well Ibidi μ-Slides. After 24 h, the cells were transiently transfected with 100 ng of GFP-M-3-P plasmid or GFP plasmid using 0.3 μL Lipofectamine 3000 and 0.3 μL P3000 reagent. After 18 h, cell media was exchanged for phenol red-free DMEM (Gibco FluoroBrite DMEM, A1896701) supplemented with 20 μM furimazine. Live cell images were captured on an Olympus IX71 microscope equipped with an Andor iXon Ultra 888 EMCCD camera and a 40× oil objective (Olympus UPlanApo 40×/1.00 oil iris). Images were captured with an EM gain of 1000, 10 MHz horizontal read-out rate, 4.33 μs vertical clock speed, and an acquisition time of 180 s. The microscope stage was kept warm with a heating pad to maintain cell viability and to encourage enzyme turnover. Fluorescence images were captured immediately following luminescence imaging, using a blue LED light source (ThorLabs, Solis-470C). Fluorescent images were captured using the EMCCD's conventional mode with an acquisition time of 0.5 s. Images were processed (removed outliers and Z-stacked) with ImageJ and colocalization was determined with the JaCoP plug-in35.

Dynamic imaging of cellular stress: HEK293T cells (5×10⁴) stably expressing RNA lanterns were plated in 8-well Ibidi μ-Slides coated with (10 mg/cm²) fibronectin. After 24 h, the cells were transiently transfected with 200 ng of plasmid encoding mCherry-β-actin with M-3-P in the 3′ UTR or CDK6-M-3-P-IRES-Staygold. Some cells were also transfected with 200 ng of plasmid encoding GFP-G3BP1 (Addgene #135997). Transfections were performed using Lipofectamine 3000 (Invitrogen, 0.4 μL) and P3000 Reagent (Invitrogen 0.4 μL). After 18 h, cell media was exchanged for phenol red-free DMEM (Gibco FluoroBrite DMEM, A1896701) supplemented with 30 μM furimazine and 0.5 mM sodium (meta)arsenite. Live cell images were captured on an Olympus IX71 microscope equipped with a HNu 512 EMCCD camera and a 40× oil objective (Olympus UPlanApo 40×/1.00 oil iris). Luminescence images were captured with an EM gain of 1000, 10 MHz horizontal read-out rate, 7.55 fps vertical clock speed, and an acquisition time of 90 s. Fluorescence images were captured immediately before and following luminescence imaging, using either a blue or green light source (Lycco Flashlight). Fluorescence emission was filtered using GFP (Olympus UM52) and Cy3 (Chroma UN41001 FITC CIN43662) filters. Fluorescence images were captured using an EM gain of 1000 with an acquisition time of 1 or 2 s. Images were processed (removed outliers and Z-stacked) with ImageJ and colocalization was determined with the JaCoP plug-in (35). Where needed, images were aligned using the Linear Stack Alignment with SIFT plugin (ref #).

Immunohistochemistry: Cells were washed with PBS (3×100 μL) and fixed using 4% PFA (RT, 10 min). The samples were then washed with PBS containing 0.025% v/v Triton X-100 (PBS-T, 3×100 μL) and permeabilized with 0.1% v/v Triton X-100 (RT, 15 min). Following permeabilization, samples were washed with PBS-T (3×100 μL) and incubated with rabbit a-G3BP1 (E9G1M) XP® (Cell Signaling Technology) in blocking buffer (1% BSA, 0.01% NaN₃, 1:100) or blocking buffer only overnight. The next day, the samples were washed with PBS-T (3×100 μL) and stained with an Alexa Fluor 594 goat a-rabbit IgG secondary antibody (Invitrogen) in a blocking buffer (1:200, 1 h, RT). Samples were washed with PBS (3×100 μL, 5 min per wash) and then imaged on a Keyence (BZ-X800) microscope with a 40× objective (Keyence Plan Apochromat 40×). Fluorescence was captured using Cy5 (BZ-X Filter Cy5) and GFP (BZ-X Filter GFP) filter cubes for G3BP1 and Staygold expression, respectively.

Imaging of doxycycline inducible RNA bait: HEK293T cells (5×10⁴) stably expressing RNA lanterns were cultured in doxycycline free media. Cells were plated in 8-well Ibidi μ-Slides coated with fibronectin (10 mg/cm²). After 24 h, the cells were transiently transfected with 200 ng TRE2-XFP-M-5-P using Lipofectamine 3000 (Invitrogen) according to the manufacturer's instructions. After 18 h, cell media was exchanged for phenol red-free DMEM (Gibco FluoroBrite DMEM, A1896701) supplemented with 30 μM furimazine and 5.12 μg/mL doxycycline hyclate. Live cell images were captured on an Olympus IX71 microscope equipped with a HNu 512 EMCCD camera and a 20× objective (Olympus LUCPlanFL 20×Ph1). Luminescence images were acquired using an EM gain of 1000, 10 MHz horizontal read-out rate, 7.55 fps vertical clock speed, and an acquisition time of 90 s. Fluorescence images were acquired immediately before and following luminescence imaging, using a green light source (Lycco Flashlight). Fluorescence emission was filtered by a Texas Red (Olympus UM52) filter. Fluorescence images were captured using an EM gain of 1000 with an acquisition time of 4 s. Images were processed (removed outliers and Z-stacked) with ImageJ and colocalization was determined with the JaCoP plug-in (35).

In vivo cell transplants: Mouse experiments were approved by the UC Irvine Animal Care and Use Committee (IACUC) in compliance with the National Institute of Health guidelines. Mice were maintained on a 12 h light/dark cycle at 25° C. All procedures were done during the light portion of the cycle. The present studies were done using Rosa26^{tdTomato} male mice obtained from the Jackson Laboratory (JAX: 007905).

Mice were anesthetized using isoflurane (1-2%) and were given subcutaneous dorsal injections of HEK293T cells (1×10⁶) expressing RNA lanterns and GFP, GFP-M-3-P, BFP-M-3-P^{mut} or BFP-M-3-P suspended in sterile PBS solution. Cells were normalized for mean fluorescence intensity (MFI) of XFP using flow cytometry. Following normalization, cells were implanted into the dorsal posterior subcutaneous flank of each mouse. Cells expressing RNA lantern and controls, GFP or BFP-M-3-P^{mut}, were implanted on the left side, while cells expressing the RNA lantern and GFP-M-3-P or BFP-M-3-P were implanted on the right side, allowing each mouse to serve as its own control. Each animal received a 20 μM furimazine injection in 200 μL of implant.

Animals were imaged immediately following transplantation. For imaging, animals were anesthetized with an i.p. injection of ketamine (6.6 mg/mL) and xylazine (1.65 mg/mL) suspended in sterile PBS and placed on a warmed (37° C.) stage. Images were captured on an Andor iXon Ultra 888 EMCCD camera equipped with an F 0.95 lens. Images were acquired with an EM gain of 1000, an acquisition time of 300 s, and signal was captured as photons. Images were processed (removing outliers and integrated density) using ImageJ. Total flux (p/s) was determined based on a given region of interest (ROI) for each implantation. Background correction was accomplished by averaging ROIs containing no luminescence. Prism (GraphPad) was used to determine significant differences (unpaired, two-tailed t-test) between groups.

### Example 2

The following is a non-limiting example of the present invention. It is to be understood that said example is not intended to limit the present invention in any way. Equivalents or substitutes are within the scope of the present invention.

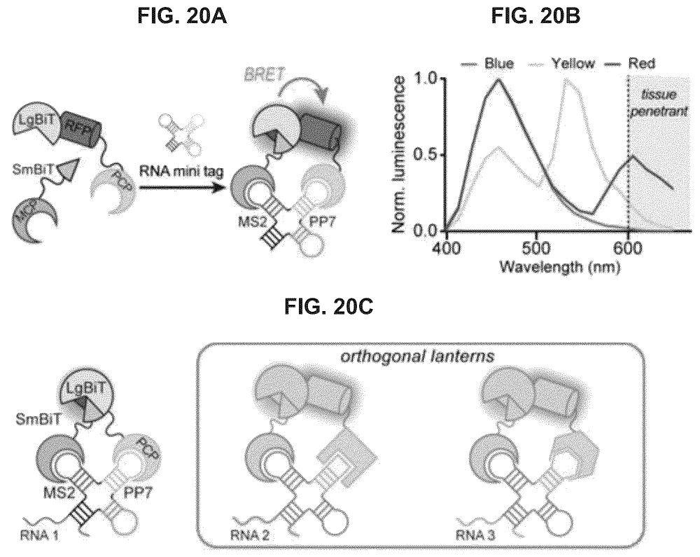

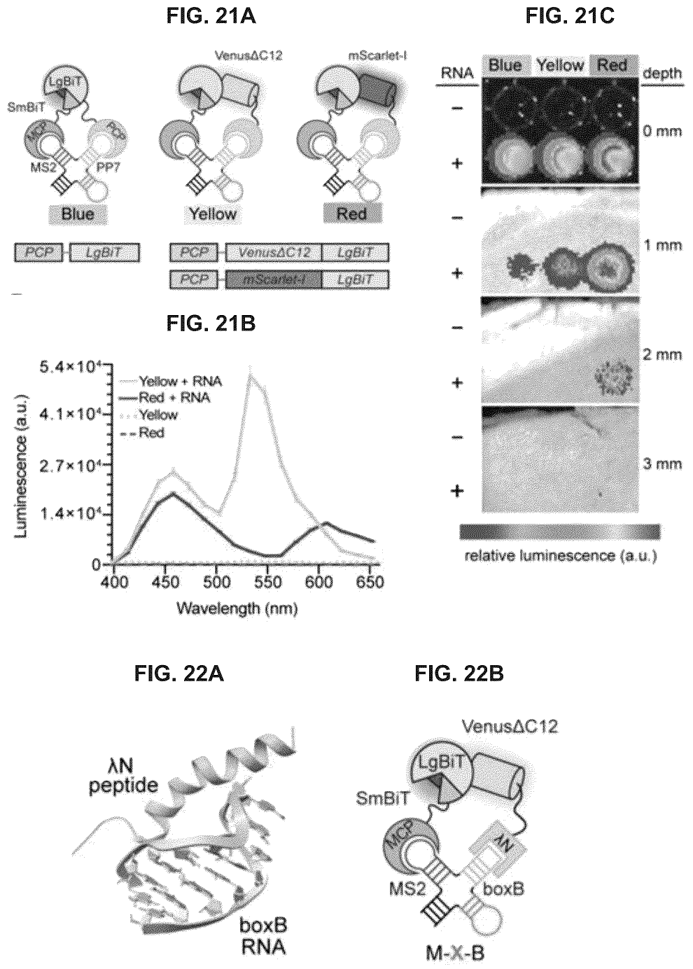

Bright and red-shifted RNA lanterns: Additional red photons can be gained via bioluminescence resonance energy transfer (BRET, FIG. 20A-20C) with luciferase-fluorescent protein fusions. BRET effectively shifts the spectrum of the emitted photons from the luciferase (blue for NanoLuc) to the emission spectrum of the fluorescent protein, such as yellow or red for Venus and mScarlet-I proteins, respectively. Far more tissue-penetrant photons are given off, enabling improved in vivo detection. Yellow and red RNA lanterns were generated by fusing Venus and mScarlet-I fluorescent proteins to the LgBiT-PCP fragment (FIG. 21A). In the presence of the M-3-P mini-tag, NanoBiT complementation resulted in red-shifted emission. Overall photon production was slightly lower than the first-generation (NanoLuc only) lantern, likely due to steric crowding associated with the larger fluorescent proteins attached. Despite the lower intensity, the BRET reporters are far more tissue penetrant (shown using different turkey bacon thickness, an excellent mammalian tissue mimic, FIG. 21B-21C). Importantly, the colors can also be distinguished using conventional filter sets on instruments and microscopes.

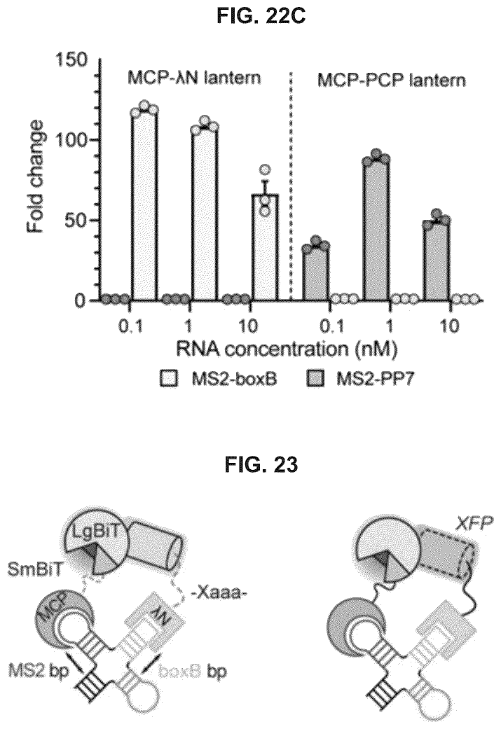

Orthogonal RNA mini-tags: For multi-transcript imaging, different colored lanterns must assemble on distinct RNA targets. Orthogonal mini-tags that can be used in conjunction with M-3-P are thus needed. Alternatives to MS2/PP7 hairpins and binding proteins have been identified and, in some cases, validated in cells. The boxB hairpin and its cognate RNA binding protein N (FIG. 22A) attracted particular interest. LgBiT was fused to N and used the corresponding boxB hairpin alongside the MS2 aptamer for transcript detection (M-X-B, FIG. 22B). The yellow lantern/M-X-B pair was readily detected in mixtures alongside the blue lantern/M-3-P pair. As shown in FIG. 22C, selective luminescence was observed for both targets (yellow versus blue). These results set an important precedence for developing and discriminating among mutually orthogonal RNA tags.

Engineer a palette of tissue-penetrant RNA lanterns and complementary mini-tags: A spectrum of red-shifted RNA lanterns were generated (via fusion to mNeonGreen, Venus, CyOFP1, or mScarlet-1) and orthogonal mini-tags (FIG. 23A-23B). The lanterns will use the same luciferin (FRZ), but yield different emission spectra (spanning blue to red). The tags will comprise combinations of the MS2, PP7, and boxB hairpins (above), in addition to aptamers CS268 and Q. These RNA motifs have a high affinity (pM-nM range) for their respective protein binders, are compatible with mammalian systems, and form simple and stable structures that are easily engineered. They are thus good candidates for a multiplexing approach. From the 5 targeted hairpins, 16 possible mini-tags are possible, as different orientations further diversify the set (e.g., M-X-P is not the same as P-X-M). Each optimized tag is expected to be <100 nt, a substantial improvement (more than an order of magnitude) over the most commonly used tags to date.

The tags will be evaluated with a preliminary set of lantern fragments in IVTT assays. Initial hits will be further screened for improved light output using panels of hairpins (FIG. 23A). These studies will be guided by structure predictions (e.g., RNAfold). Lantern fragments with different linker lengths between the RNA binding protein and luciferase fragment will also be tested (FIG. 23B), to maximize photon output and tissue penetrance.

### Embodiments

The following embodiments are intended to be illustrative only and not to be limiting in any way.

Embodiment 1: A bioluminescent composition for visualizing RNA dynamics comprising an RNA comprising at least a first stem-loop, wherein the first stem-loop is bound by a first RNA-interacting protein linked to a first portion of a detectable reporter.

Embodiment 2: The composition of embodiment 1, further comprising a second stem-loop, wherein the second stem-loop is bound by a second RNA-interacting protein linked to a second portion of a detectable reporter.

Embodiment 3: The composition of embodiment 1 or embodiment 2, wherein the detectable reporter comprises a light emitting luciferase protein or proximity labeling protein.

Embodiment 4: The composition of any one of embodiments 1-3, wherein the first or second RNA-interacting protein comprises an MS2 RNA-binding protein, a PP7 RNA binding protein, an L7Ae RNA binding protein, or a lambdaN RNA binding protein.

Embodiment 5: The composition of embodiment 3 or embodiment 4, wherein the first portion of the light emitting luciferase protein comprises an N-terminal fragment of the light emitting luciferase protein, wherein the MS2 RNA binding protein is linked to the N-terminal fragment of the light emitting luciferase protein.

Embodiment 6: The composition of embodiment 5, wherein the MS2 RNA binding protein is linked to the N-terminal fragment via a linker.

Embodiment 7: The composition of embodiment 3 or embodiment 4, wherein the second portion of the light emitting luciferase protein comprises a C-terminal fragment of the light emitting luciferase protein, wherein the PP7 RNA binding protein is linked to the C-terminal fragment of the light emitting luciferase protein.

Embodiment 8: The composition of embodiment 7, wherein the PP7 RNA binding protein is linked to the C-terminal fragment via a linker.

Embodiment 9: The composition of embodiment 6 or embodiment 8, wherein the linker is short, hydrophobic, or a combination thereof.

Embodiment 10: The composition of any one of embodiments 1-9, wherein the detectable reporter becomes functional when the first portion and the second portion are brought together.

Embodiment 11: The composition of any one of embodiments 1-10, wherein the detectable reporter comprises a light emitting luciferase protein, wherein the light emitting luciferase protein becomes functional when the first portion and the second portion are brought together.

Embodiment 12: The composition of any one of embodiments 1-11, wherein a 5′ end of the RNA and the a 3′ end of the RNA are complementary and form an RNA stem, wherein the first stem-loop and the second stem-loop are between the complementary 5′ end and the 3′ end of the RNA.

Embodiment 13: The composition of embodiment 12, wherein the composition comprises a structured RNA, wherein a structure RNA refers to an RNA comprising extensive base pairing, tertiary interactions, or a combination thereof.

Embodiment 14: The composition of any one of embodiments 1-11, wherein a 5′ end of the RNA and a 3′ end of the RNA are non-complementary and form single-stranded RNA (ssRNA) arms, wherein the ssRNA arms flank the first stem-loop and the second stem-loop.

Embodiment 15: The composition of embodiment 14, wherein the composition is unstructured until a target RNA base pairs with the ssRNA arms, wherein when the target RNA binds to the ssRNA arms, a structure RNA is formed, wherein a structure RNA refers to an RNA comprising extensive base pairing, tertiary interactions, quaternary interactions, or a combination thereof.

Embodiment 16: The composition of embodiment 15, wherein the target RNA comprises a cellular RNA.

Embodiment 17: The composition of embodiment 16, wherein the cellular RNA comprises a messenger RNA (mRNA), a splice variant of an mRNA, a microRNA, or a non-coding RNA (ncRNA).

Embodiment 18: The composition of any one of embodiments 1-17, wherein the composition comprises a structured RNA formed by trans-splicing, RNA editing, or DNA editing, wherein a structure RNA refers to an RNA comprising extensive base pairing, tertiary interactions, or a combination thereof.

Embodiment 19: The composition of any one of embodiments 1-18, further comprising a third stem-loop, wherein the third stem-loop is downstream of the first and the second stem-loop.

Embodiment 20: The composition of embodiment 19, wherein the third stem-loop binds to a detectable reporter.

Embodiment 21: The composition of embodiment 20, wherein the detectable reporter comprises an optical reporter.

Embodiment 22: The composition of embodiment 21, wherein the optical reporter comprises a fluorescent reporter.

Embodiment 23: The composition of embodiment 22, wherein the fluorescent reporter comprises a yellow fluorescent protein (YFP) or a red fluorescent protein (RFP).

Embodiment 24: The composition of any one of embodiments 19-23, wherein when the RNA undergoes 3′ RNA decay by a 3′ exonuclease, the third stem-loop will be digested by the 3′ exonuclease and luminescences from the functional light emitting luciferase protein will be observed.

Embodiment 25: The composition of embodiment 1, wherein the first stem-loop is at or near the 3′ end of the RNA.

Embodiment 26: The composition of embodiment 25, further comprising a circular RNA comprising a stem-loop and a polyU sequence, wherein the polyU sequence binds to at least a portion of a polyA sequence of the RNA, wherein the stem-loop is bound by a second RNA-interacting protein linked to a second portion of a detectable reporter.

Embodiment 27: The composition of embodiment 25 or embodiment 26, wherein the detectable reporter comprises a light emitting luciferase protein or proximity labeling protein.

Embodiment 28: The composition of any one of embodiments 25-28, wherein the first or second RNA-interacting protein comprises an MS2 RNA-binding protein, a PP7 RNA binding protein, a L7Ae RNA binding protein, or a lambdaN RNA binding protein.

Embodiment 29: The composition of any one of embodiments 25-28, wherein the MS2-RNA binding protein binds to the first stem-loop and the PP7 RNA binding protein binds to the stem-loop of the circular RNA.

Embodiment 30: The composition of any one of embodiments 25-29, wherein the detectable reporter becomes functional when the first portion and the second portion are brought together.

Embodiment 31: The composition of any one of embodiments 25-30, wherein the detectable reporter comprises a light emitting luciferase protein, wherein the light emitting luciferase protein becomes functional when the first portion and the second portion are brought together.

Embodiment 32: The composition of embodiment 31, wherein when first portion and the second portion are brought together the functional luciferase protein is cleaved from the RNA-interacting proteins.

Embodiment 33: The composition of any one of embodiments 25-32, wherein the composition allows for detection of mRNAs splicing or polyadenylation.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A bioluminescent composition for visualizing RNA dynamics comprising an RNA comprising at least a first stem-loop and a second stem-loop, wherein the first stem-loop is bound by a first RNA-interacting protein linked to a first portion of a detectable reporter; and wherein the second stem-loop is bound by a second RNA-interacting protein linked to a second portion of a detectable reporter.

2. The composition of claim 1, wherein the first or second RNA-interacting protein comprises an MS2 RNA-binding protein, a PP7 RNA binding protein, an L7Ae RNA binding protein, or a lambdaN RNA binding protein.

3. The composition of claim 1, wherein the detectable reporter comprising a light emitting luciferase protein or proximity labeling protein.

4. The composition of claim 1, wherein the first portion of the light emitting luciferase protein comprises an N-terminal fragment of the light emitting luciferase protein, wherein the MS2 RNA binding protein is linked to an N-terminal fragment of the light emitting luciferase protein.

5. The composition of claim 4, wherein the MS2 RNA binding protein is linked to the N-terminal fragment via a linker.

6. The composition of claim 1, wherein the second portion of the light emitting luciferase protein comprises a C-terminal fragment of the light emitting luciferase protein, wherein the PP7 RNA binding protein is linked to the C-terminal fragment of the light emitting luciferase protein.

7. The composition of claim 6, wherein the PP7 RNA binding protein is linked to the C-terminal fragment via a linker.

8. The composition of claim 1, wherein the detectable reporter becomes functional when the first portion and the second portion are brought together.

9. The composition of claim 1, wherein a 5′ end of the RNA and a 3′ end of the RNA are complementary and form an RNA stem, wherein the first stem-loop and the second stem-loop are between the complementary 5′ end and the 3′ end of the RNA.

10. The composition of claim 1, wherein a 5′ end of the RNA and a 3′ end of the RNA are non-complementary and form single-stranded RNA (ssRNA) arms, wherein the ssRNA arms flank the first stem-loop and the second stem-loop.

11. The composition of claim 10, wherein the composition is unstructured until a target RNA base pairs with the ssRNA arms, wherein when the target RNA binds to the ssRNA arms, a structure RNA is formed, wherein a structure RNA refers to an RNA comprising extensive base-pairing, tertiary interactions, quaternary interactions, or a combination thereof.

12. The composition of claim 11, wherein the target RNA comprises a cellular RNA.

13. The composition of claim 12, wherein the cellular RNA comprises a messenger RNA (mRNA), a splice variant of an mRNA, a microRNA, or a non-coding RNA (ncRNA).

14. The composition of claim 1, wherein the composition comprises a structured RNA formed by trans-splicing, RNA editing, or DNA editing, wherein a structure RNA refers to an RNA comprising extensive base pairing, tertiary interactions, or a combination thereof.

15. The composition of claim 1, the RNA further comprises a third stem-loop, wherein the third stem-loop is downstream of the first and the second stem-loop.

16. The composition of claim 15, wherein the third stem-loop binds to a second detectable reporter.


SEQUENCE LISTING (41 sequences)

SEQ ID NO: 1 - DNA, 2596 bp, synthetic construct
  source 1..2596: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgcggattctagacgggatcccgccaccgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccaccggtatgcatcgccaccgccttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgat

SEQ ID NO: 2 - DNA, 2586 bp, synthetic construct
  source 1..2586: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgcggattctagacgggatcccgccaccgtgaccggctaccgcctgtttgagaaaattagctaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccaccggtatgcatcgccaccgccttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaa

SEQ ID NO: 3 - DNA, 2683 bp, synthetic construct
  source 1..2683: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgcggattctagacgggatcccgccaccgtgaccggctaccgcctgtttgagaaaattagcgggagcggaagtggggttaccggttatcgtctgttcgaaaaaatcagcgggtccggatcgggtgtgacaggctatcgtctgtttgagaaaatttcctaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccaccggtatgcatcgccaccgccttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 4 - DNA, 2683 bp, synthetic construct
  source 1..2683: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgcggattctagacgggatcccgccaccgtgaccggctaccgcctgtttgaggagattctggggagcggaagtggggtgaccggatatcggctgtttgaggagattctggggtccggatcgggtgtgacagggtatcgcctgttcgaggaaatcctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccaccggtatgcatcgccaccgccttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 5 - DNA, 2572 bp, synthetic construct
  source 1..2572: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgatatcggcggcggaggctctgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtatcgatggcggaggcgggtctttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 6 - DNA, 2602 bp, synthetic construct
  source 1..2602: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgatatcggcggcggaggctctgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtatcgatggcggaggcgggtctggcggcggcggcagcggtggaggaggttctttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 7 - DNA, 2632 bp, synthetic construct
  source 1..2632: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgatatcggcggcggaggctctgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtatcgatggcggaggcgggtctggtggagggggatctggaggtggtgggtctggcggcggcggcagcggtggaggaggttctttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 8 - DNA, 2602 bp, synthetic construct
  source 1..2602: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgatatcggcggcggaggctctggaggcggcggaagcggtgggggtgggagcgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtatcgatggcggaggcgggtctttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 9 - DNA, 2632 bp, synthetic construct
  source 1..2632: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgatatcggcggcggaggctctggaggcggcggaagcggtgggggtgggagcgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtatcgatggcggaggcgggtctggcggcggcggcagcggtggaggaggttctttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 10 - DNA, 2662 bp, synthetic construct
  source 1..2662: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgatatcggcggcggaggctctggaggcggcggaagcggtgggggtgggagcgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtatcgatggcggaggcgggtctggtggagggggatctggaggtggtgggtctggcggcggcggcagcggtggaggaggttctttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 11 - DNA, 2632 bp, synthetic construct
  source 1..2632: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgatatcggcggcggaggctctggtggaggaggttctggcggcggtggttccggaggcggcggaagcggtgggggtgggagcgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtatcgatggcggaggcgggtctttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 12 - DNA, 2662 bp, synthetic construct
  source 1..2662: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgatatcggcggcggaggctctggtggaggaggttctggcggcggtggttccggaggcggcggaagcggtgggggtgggagcgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtatcgatggcggaggcgggtctggcggcggcggcagcggtggaggaggttctttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 13 - DNA, 2692 bp, synthetic construct
  source 1..2692: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgatatcggcggcggaggctctggtggaggaggttctggcggcggtggttccggaggcggcggaagcggtgggggtgggagcgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtatcgatggcggaggcgggtctggtggagggggatctggaggtggtgggtctggcggcggcggcagcggtggaggaggttctttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 14 - DNA, 3271 bp, synthetic construct
  source 1..3271: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgcggattctagacgggatcccgccaccgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccaccggtatgcatcgccaccgccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgcgatgttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 15 - DNA, 3262 bp, synthetic construct
  source 1..3262: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgcggattctagacgggatcccgccaccgtgaccggctaccgcctgtttgaggagattctgtaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccaccggtatgcatcgccaccgccgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggaaagttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcctcgaggactacaaggacgacgatgacaag

SEQ ID NO: 16 - DNA, 3751 bp, synthetic construct
  source 1..3751: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagagccaccatgggcccaaaaaagaaaagaaaagttggctacccctacgacgtgcccgactacgccatcgaaggccgccatatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgcggattctagacgggatcccgccaccgaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtcctaacgtacgacgcgtggatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagcctgcaggatgggctccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgcgtcaacctaaaactggatcaggcggacgtcgttgattccggacttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgtgcggatccaccggtatgcatcgccaccgcccctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgctcgaggactacaaggacgacgatgacaag

SEQ ID NO: 17 - DNA, 1396 bp, synthetic construct
  source 1..1396: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcgccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagaattctgcagatatccatcacactggcggccgcgggagacgagcatcagccgtcgagagcagacgatatggcgtcgctcggcaattgaaaaaacgccctccc

SEQ ID NO: 18 - DNA, 1395 bp, synthetic construct
  source 1..1395: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcgccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagaattctgcagatatccatcacactggcggccgcgggagacgagcatcagccgtcgagagcgacgttatggcgtcgctcggcaattgaaaaaacgccctccc

SEQ ID NO: 19 - DNA, 1476 bp, synthetic construct
  source 1..1476: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcggccgccaccatgctgctgcccgtccccctgctgctgggcctgctgggcgccgccgcggatccgatggtgagcaagggcgaggagctgtttacaggcgtgacccccttcaagttccagctgaagggcaccatcaacggcaagagcttcaccgtggaaggcgagggcgagggcaatagccacgagggcagccacaaaggcaagtacgtgtgcaccagcggcaaactgccaatgtcttgggccgccctgggaactagcttcggctatggcatgaagtactacaccaagtaccccagcggcctgaagaactggttccacgaggtgatgcccgagggcttcacctacgacagacacatccagtacaagggcgacggcagcatccacgccaagcaccagcacttcatgaagaacggcacctaccacaacatcgtggagttcaccggccaggacttcaaggagaacagccccgtgctgaccggcgacatgaacgtgagcctgcccaacgaggtgcagcacatccccagagatgacggcgtggagtgcccagtgaccctgctgtaccctctgctgagcgacaagagcaagtgcgtggaggcctaccagaacaccatcatcaagcccctgcacaatcagccagcccccgatgtgccataccactggatcagaaagcagtacacccagagcaaggacgacaccgaggagagagaccacatcatccagagcgagaccctggaggcccacctgccatggcacgagccttctgcttctgccgtgaaggatgaactgtaagaattctgcagatatccatcacactggcggccgcgggagacgagcatcagccgtcgagagcagacgatatggcgtcgctcggcaattgaaaaaacgccctccc

SEQ ID NO: 20 - DNA, 1235 bp, synthetic construct
  source 1..1235: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatggcgtctactcccttcaagttccagctgaagggcaccatcaacggcaagagcttcaccgtggaaggcgagggcgagggcaatagccacgagggcagccacaaaggcaagtacgtgtgcaccagcggcaaactgccaatgtcttgggccgccctgggaactagcttcggctatggcatgaagtactacaccaagtaccccagcggcctgaagaactggttccacgaggtgatgcccgagggcttcacctacgacagacacatccagtacaagggcgacggcagcatccacgccaagcaccagcacttcatgaagaacggcacctaccacaacatcgtggagttcaccggccaggacttcaaggagaacagccccgtgctgaccggcgacatgaacgtgagcctgcccaacgaggtgcagcacatccccagagatgacggcgtggagtgcccagtgaccctgctgtaccctctgctgagcgacaagagcaagtgcgtggaggcctaccagaacaccatcatcaagcccctgcacaatcagccagcccccgatgtgccataccactggatcagaaagcagtacacccagagcaaggacgacaccgaggagagagaccacatcatccagagcgagaccctggaggcccacctg

SEQ ID NO: 21 - DNA, 83 bp, synthetic construct
  source 1..83: mol_type = other DNA
gtaatacgactcactataggcacgagcatcagccgtgcctccaggtcgaatcttcaaacgagcagacgatatggcgtcgctcg

SEQ ID NO: 22 - DNA, 39 bp, synthetic construct
  source 1..39: mol_type = other DNA
gtaatacgactcactataggcacgagcatcagccgtgcc

SEQ ID NO: 23 - DNA, 43 bp, synthetic construct
  source 1..43: mol_type = other DNA
gtaatacgactcactataggagcagacgatatggcgtcgctcg

SEQ ID NO: 24 - DNA, 86 bp, synthetic construct
  source 1..86: mol_type = other DNA
gtaatacgactcactatagggagacgagcatcagccgtcgagagcgacgttatggcgtcgctcggcaattgaaaaaacgccctccc

SEQ ID NO: 25 - DNA, 84 bp, synthetic construct
  source 1..84: mol_type = other DNA
gtaatacgactcactatagggacacgagcatcagccgtggagcagacgatatggcgtcgctcgcgaattgaaaaaaccgctccc

SEQ ID NO: 26 - DNA, 85 bp, synthetic construct
  source 1..85: mol_type = other DNA
gtaatacgactcactatagggacgcgagcatcagccgcgagagcagacgatatggcgtcgctcgcgaattgaaaaaaccgctccc

SEQ ID NO: 27 - DNA, 87 bp, synthetic construct
  source 1..87: mol_type = other DNA
gtaatacgactcactatagggagacgagcatcagccgtcgagagcagacgatatggcgtcgctcggcaattgaaaaaacgccctccc

SEQ ID NO: 28 - DNA, 89 bp, synthetic construct
  source 1..89: mol_type = other DNA
gtaatacgactcactatagggagacgagcatcagccgtcggagagcagacgatatggcgtcgctcggcaattgaaaaaacgcccctccc

SEQ ID NO: 29 - DNA, 91 bp, synthetic construct
  source 1..91: mol_type = other DNA
gtaatacgactcactatagggagacgagcatcagccgtccggagagcagacgatatggcgtcgctcggcaattgaaaaaacgccccgtccc

SEQ ID NO: 30 - DNA, 93 bp, synthetic construct
  source 1..93: mol_type = other DNA
gtaatacgactcactatagggagacgagcatcagccgtccgagagagcagacgatatggcgtcgctcggcaattgaaaaaacgccctcgtccc

SEQ ID NO: 31 - DNA, 95 bp, synthetic construct
  source 1..95: mol_type = other DNA
gtaatacgactcactatagggagacgagcatcagccgtccgacgagagcagacgatatggcgtcgctcggcaattgaaaaaacgcccgtcgtccc

SEQ ID NO: 32 - DNA, 96 bp, synthetic construct
  source 1..96: mol_type = other DNA
gtaatacgactcactatagggagacgagcatcagccgtcctgagcggcaattgaaaaaacgccgagcagacgatatggcgtcgctcgctcagtccc

SEQ ID NO: 33 - DNA, 105 bp, synthetic construct
  source 1..105: mol_type = other DNA
gtaatacgactcactatagggagacgagcatcagccgtctcgacgagcaagagcagacgatatggcgtcgctcggcaattgaaaaaacgcctgctcgtcgatccc

SEQ ID NO: 34 - DNA, 93 bp, synthetic construct
  source 1..93: mol_type = other DNA
gtaatacgactcactatagggacgacgagcatcagccgtcgcggagagcagacgatatggcgtcgctcggcaattgaaaaaacgccccgtccc

SEQ ID NO: 35 - DNA, 95 bp, synthetic construct
  source 1..95: mol_type = other DNA
gtaatacgactcactatagggacgactgagcatcagccagtcgcggagagcagacgatatggcgtcgctcggcaattgaaaaaacgccccgtccc

SEQ ID NO: 36 - DNA, 89 bp, synthetic construct
  source 1..89: mol_type = other DNA
gtaatacgactcactatagggacgacgagcatcagccgtcggagagcagacgatatggcgtcgctcggcaattgaaaaaacgccctccc

SEQ ID NO: 37 - DNA, 91 bp, synthetic construct
  source 1..91: mol_type = other DNA
gtaatacgactcactatagggacgactgagcatcagccagtcggagagcagacgatatggcgtcgctcggcaattgaaaaaacgccctccc

SEQ ID NO: 38 - DNA, 83 bp, synthetic construct
  source 1..83: mol_type = other DNA
gtaatacgactcactatagggagacgagcatcagccgtcgagcagacgatatggcgtcgcggcaattgaaaaaacgccctccc

SEQ ID NO: 39 - DNA, 89 bp, synthetic construct
  source 1..89: mol_type = other DNA
gtaatacgactcactatagggagacgagcatcagccgtcgagagcgagacgatatggcgtccgctcggcaattgaaaaaacgccctccc

SEQ ID NO: 40 - DNA, 91 bp, synthetic construct
  source 1..91: mol_type = other DNA
gtaatacgactcactatagggagacgagcatcagccgtcgagagcgcagacgatatggcgtcgcgctcggcaattgaaaaaacgccctccc

SEQ ID NO: 41 - DNA, 93 bp, synthetic construct
  source 1..93: mol_type = other DNA
gtaatacgactcactatagggagagcagacgatatggcgtcgctcccagacacgagcatcagccgtgggcaattgaaaaaacgccctggtccc